(19) United States
(12) Patent Application Publication
(10) Pub. No.: US 2023/0128252 A1
(43) Pub. Date: Apr. 27, 2023

(54) PHARMACEUTICAL COMPOSITIONS

(71) Applicant: Shenzhen Pharmacin Co., Ltd., Shenzhen (CN)

(72) Inventors: Zeren WANG, Southbury, CT (US); Jun XU, Newtown, CT (US); Peter FARINA, North Salem, NY (US); Shun CHEN, Shenzhen (CN)

(21) Appl. No.: 17/963,285

(22) Filed: Oct. 11, 2022

Related U.S. Application Data
(63) Continuation of application No. PCT/CN2021/087690, filed on Apr. 16, 2021.

(30) Foreign Application Priority Data
Apr. 17, 2020 (CN) PCT/CN2020/085241

(51) Int. Cl. A61K 31/517; A61K 47/06; A61K 9/00; A61P 17/00

(52) U.S. Cl. CPC A61K 31/517; A61K 47/06; A61K 9/0014; A61P 17/00

(57) ABSTRACT

Provided are pharmaceutical compositions which include an active pharmaceutical ingredient having a calculated log P in octanol-water equal or greater than about 4.0, at least one solubilizer, and optionally at least one stratum corneum penetration enhancer, wherein the pharmaceutical composition can be formulated for topical administration. In some instances, the active pharmaceutical ingredient is clofazimine.

### CROSS REFERENCE TO RELATED APPLICATIONS

This application is a continuation of PCT Application No. PCT/CN2021/087690, filed Apr. 16, 2021, which claims the benefit of PCT Application No. PCT/CN2020/085241, filed Apr. 17, 2020, each of which is hereby incorporated by reference in its entirety.

### BACKGROUND

Lipophilic active pharmaceutical ingredients such as clofazimine have limited solubilities in aqueous conditions. Consequently, the adsorption and bioavailability of the lipophilic ingredients can be limited by its dissolution. Exemplary lipophilic active pharmaceutical ingredients include Hesperetin, Lormetazepam, Naringenin, Cinchonidine, Alprenolol, Propranolol, Ketoprofen, Clofibric acid, clofazimine, Naproxen, Warfarin, Apigenin, Diazepam, Quinine, Quetiapine, Rosiglitazone, Clotiazepam, Tramadol, Fenbufen, Chlorphenamine, Pyrilamine, Venlafaxine, Brompheniramine, Diphenhydramine, Chrysin, Valsartan, Penbutolol, Diltiazem, Ibuprofen, Bupivacaine, Flurbiprofen, Progesterone, Chlordiazepoxide, Trazodone, Haloperidol, Glimepiride, Abiraterone, Indometacin, Clopidogrel, Flurazepam, Duloxetine, Nortriptyline, Celecoxib, Nilotinib, Atorvastatin, Maprotiline, Fluoxetine, Diclofenac, Amitriptyline, Imipramine, Loratadine, Cyproheptadine, Chlorpromazine, Sertraline, Flufenamic acid, Miconazole, and Rimonabant.

One of the exemplary lipophilic ingredients is Clofazimine, chemical name N,5-bis(4-chlorophenyl)-3-propan-2-ylimino phenazin-2-amine. Clofazimine is a fat soluble, brick red phenazine dye. It is an anti-infective agent with anti-mycobacterial activity.

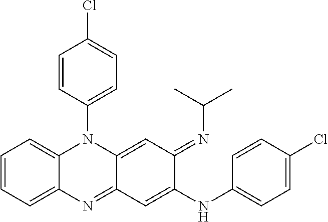 [SMILES: CC(C)/N=c1\cc2n(-c3ccc(Cl)cc3)c3ccccc3nc-2cc1Nc1ccc(Cl)cc1]

C₂₇H₂₂Cl₂N₄
Molecular Weight=473.40
pKa=8.37
Log P=7.48
Water solubility=0.225 mg/L
(virtually insoluble)

Clofazimine

Clofazimine, administered orally, is the first line of therapy for leprosy (in combination with rifampicin and dapsone), and provides a high rate of ultimate cure after 1 to 3 years of treatment. Clofazimine was approved for use in the United States in 1986, was withdrawn in 2016, and is now only available under the auspices of the National Hansen's Disease Program. Clofazimine, marketed under brand name Lamprene, is available as 50 mg soft capsules, also containing beeswax, butylated hydroxytoluene, citric acid, ethyl vanillin, gelatin, glycerin, iron oxide, lecithin, p-methoxy acetophenone, parabens, plant oils, propylene glycol, in which clofazimine is dispersed as micron-sized particles. According to the FDA Label information, clofazimine has a variable absorption rate, ranging from 45-62%. Food effects after oral administration were observed, wherein time to reach peak plasma concentration (Tₘₐₓ) decreases from 12 hours to 8 hours under fed conditions relative to the fasted state. Prescribing instructions are to take Lamprene with meals.

The exact mechanism through which clofazimine acts is unknown. However, it binds preferentially to mycobacterial DNA, thereby inhibiting DNA replication and cell growth. It also increases activity of bacterial phospholipase A2, leading to release and accumulation of lysophospholipids, which are toxic and inhibit bacterial proliferation.

The major side effects of clofazimine include skin discoloration and gastrointestinal upset with pain, nausea and diarrhea. The skin discoloration is due to the reddish-orange color of clofazimine and results in a pinkish-brown discoloration of skin and bodily fluids in the majority of patients treated for more than a month. The discoloration fades with cessation of the drug, but may persist for months or years. The gastrointestinal side effects of clofazimine can be severe and require dose modification or discontinuation. The symptoms appear to be due to crystallization of the clofazimine molecule in intestinal submucosa; these crystals can also be found in liver, lymph nodes and spleen. The possible causes of liver injury from clofazimine therapy are not known and might relate to formation of drug-crystals in macrophages in the liver. While crystals of clofazimine can be found in liver and spleen in patients on prolonged therapy, they do not appear to be associated with appreciable liver damage.

Clofazimine also demonstrates immunosuppressive and anti-inflammatory activity. Clofazimine is a Kv1.3 blocker. The KCNA3 gene encodes the potassium voltage-gated channel Kv1.3 (or KCNA3) protein expressed in T and B lymphocytes and plays an essential role in T lymphocyte effector memory (TEM) cell activation and proliferation. Selective suppression of effector-memory T cells with a Kv1.3-specific blocker could address many autoimmune diseases, (e.g., multiple sclerosis, rheumatoid arthritis, type 1 diabetes) without compromising the protective immune response. In proof-of-concept studies, Kv1.3 blockers have prevented and treated disease in rat models of multiple sclerosis, type-1 diabetes mellitus, rheumatoid arthritis, contact dermatitis, and delayed-type hypersensitivity. In a human clinical study, a majority of patients showed remission in treating chronic discoid lupus erythematosus with Clofazimine. Other clinical studies have shown promise in treating other autoimmune diseases, such as psoriasis, Miescher's granulomatous cheilitis, with Clofazimine.

For the treatment of skin disease, topical formulations are often preferable. However, as a natural protective layer for the human body, the outer layer of the skin (the stratum corneum), significantly impedes drug penetration. Despite the above mentioned therapeutic successes of clofazimine, topical formulations are not available. Clofazimine is significantly hydrophobic presenting challenges in development as a topical formulation for clinical use.

Accordingly, there is a need for pharmaceutical compositions comprising clofazimine formulated for topical administration. More generally, there is a need for improved formulations of highly lipophilic active pharmaceutical ingredients, for example, for topical administration. There is an unmet need to provide a therapy to treat, prevent, reduce the severity of, reduce the incidence of, delay the onset of, or reduce the pathogenesis of skin diseases including psoriasis.

### INCORPORATION BY REFERENCE

All publications and patent applications mentioned in this specification are herein incorporated by reference to the same extent as if each individual publication or patent application was specifically and individually indicated to be incorporated by reference.

### BRIEF SUMMARY

In one aspect, described herein are stable topical formulations comprising a lipophilic active pharmaceutical ingredient, e.g., an API of table 1. It was discovered by the inventors of the present disclosure that topical pharmaceutical formulations described herein provide improved chemical and physical stability for the API.

Provided herein are pharmaceutical compositions, and methods for using said compositions for the treatment of diseases. In one aspect, provided herein is a topical pharmaceutical composition that comprises: (a) an active pharmaceutical ingredient (API) or a pharmaceutically acceptable salt thereof present in an amount of up to about 2% of the total weight of the composition, wherein the active pharmaceutical ingredient has a calculated log P in octanol-water equal or greater than about 4.0; and (b) at least one solubilizer, wherein the pharmaceutical composition is formulated for topical administration. In some embodiments, the API or the pharmaceutically acceptable salt thereof is dissolved in the at least one solubilizer. In one aspect, provided herein is a topical pharmaceutical composition that comprises: (a) an active pharmaceutical ingredient (API) or a pharmaceutically acceptable salt thereof present in an amount of up to about 10.0% of the total weight of the composition, wherein the active pharmaceutical ingredient has a calculated log P in octanol-water equal or greater than about 4.0; and (b) at least one solubilizer, wherein the API or the pharmaceutically acceptable salt thereof is dissolved in the at least one solubilizer, and wherein the pharmaceutical composition is formulated for topical administration. In some embodiments, the API or the pharmaceutically acceptable salt thereof is present in an amount of up to about 5% or 2% of the total weight of the composition. In some embodiments, the API or the pharmaceutically acceptable salt thereof is present in an amount of up to about 1.5% of the total weight of the composition. In some embodiments, the API or the pharmaceutically acceptable salt thereof is present in an amount of up to about 1% of the total weight of the composition. In some embodiments, the API or the pharmaceutically acceptable salt thereof is present in an amount of from about 0.001% to about 1% of the total weight of the pharmaceutical composition. In some embodiments, the API or the pharmaceutically acceptable salt thereof is present in an amount of from about 0.01% to about 0.1% of the total weight of the pharmaceutical composition. In some embodiments, the API or the pharmaceutically acceptable salt thereof is present in an amount of from about 0.01% to about 1% of the total weight of the pharmaceutical composition. In some embodiments, the API or the pharmaceutically acceptable salt thereof is present in an amount of from about 0.005% to about 0.5% of the total weight of the pharmaceutical composition. In some embodiments, the API or the pharmaceutically acceptable salt thereof is present in an amount of from about 0.05% to about 0.5% of the total weight of the pharmaceutical composition. In some embodiments, the API or the pharmaceutically acceptable salt thereof is present in an amount of from about 0.1% to about 0.2% of the total weight of the pharmaceutical composition. In some embodiments, the API or the pharmaceutically acceptable salt thereof is present in an amount of from about 0.1% to about 0.5% of the total weight of the pharmaceutical composition. In some embodiments, the API is Hesperetin, Lormetazepam, Naringenin, Cinchonidine, Alprenolol, Propranolol, Ketoprofen, Clofibric acid, Naproxen, Warfarin, Apigenin, Diazepam, Quinine, Quetiapine, Rosiglitazone, Clotiazepam, Tramadol, Fenbufen, Chlorphenamine, Pyrilamine, Venlafaxine, Brompheniramine, Diphenhydramine, Chrysin, Valsartan, Penbutolol, Diltiazem, Ibuprofen, Bupivacaine, Flurbiprofen, Progesterone, Chlordiazepoxide, Trazodone, Haloperidol, Glimepiride, Abiraterone, Indometacin, Clopidogrel, Flurazepam, Duloxetine, Nortriptyline, Celecoxib, Nilotinib, Atorvastatin, Maprotiline, Fluoxetine, Diclofenac, Amitriptyline, Imipramine, Loratadine, Cyproheptadine, Chlorpromazine, Sertraline, Flufenamic acid, Miconazole or Rimonabant. In some embodiments, the API has a calculated log P in octanol-water equal or greater than about 4.5, 5.0, 5.5, 6.0, 6.5, 7.0, or 7.5. In some embodiments, the API has a calculated log P in octanol-water within a range of from about 4.0, 4.5, 5.0, 5.5 or 6 to about 7, 8, 9, or 10. In some embodiments, the pharmaceutical composition is non-aqueous. In one aspect, provided herein is a topical pharmaceutical composition that comprises: (a) a compound or a pharmaceutically acceptable salt thereof, present in an amount of up to about 2% of the total weight of the composition, wherein the compound has a structure of Formula I:

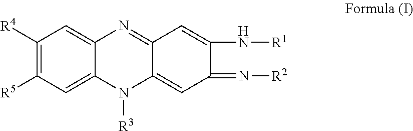

wherein,
each of R¹, R² and R³ is independently an aryl radical selected from the group consisting of phenyl, chlorophenyl, lower alkylphenyl and lower alkoxyphenyl; a C₁-C₁₂ alkyl; a C₃-C₈ cycloalkyl; or a C₁-C₁₁ heteroalkyl, wherein each of the aryl, alkyl, cycloalkyl, and heteroalkyl is substituted or unsubstituted;
R₄ represents a hydrogen or halogen atom; and
R₅ represents a hydrogen or halogen atom;

and

(b) at least one solubilizer,

wherein the pharmaceutical composition is formulated for topical administration. In some embodiments, the compound or the pharmaceutically acceptable salt thereof is dissolved in the at least one solubilizer. In one aspect, provided herein is a topical pharmaceutical composition that comprises: (a) a compound or a pharmaceutically acceptable salt thereof, present in an amount of up to about 10.0% of the total weight of the composition, wherein the compound has a structure of Formula I:

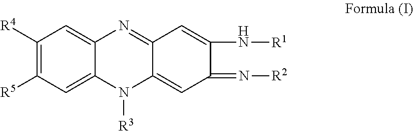

wherein,
each of R¹, R² and R³ is independently an aryl radical selected from the group consisting of phenyl, chlorophenyl, lower alkylphenyl and lower alkoxyphenyl; a C₁-C₁₂ alkyl; a C₃-C₈ cycloalkyl; or a C₁-C₁₁ heteroalkyl, wherein each of the aryl, alkyl, cycloalkyl, and heteroalkyl is substituted or unsubstituted;
R₄ represents a hydrogen or halogen atom; and
R₅ represents a hydrogen or halogen atom;

and

(b) at least one solubilizer,

wherein the compound or the pharmaceutically acceptable salt thereof is dissolved in the at least one solubilizer, and wherein the pharmaceutical composition is formulated for topical administration. In some embodiments, R¹ is phenyl, chlorophenyl, lower alkylphenyl, or lower alkoxyphenyl. In some embodiments, R³ is phenyl, chlorophenyl, lower alkylphenyl, or lower alkoxyphenyl. In some embodiments, R² is an ethyl, propyl, butyl, pentyl, hexyl, heptyl, decyl, cyclohexyl, or cycloheptyl radical. In some embodiments, R² is an ethyl, n-propyl, 1-methylethyl (i-propyl), n-butyl, heptyl, 1,3-dimethylbutyl, sec-butyl, 1,1-dimethylethyl (t-butyl), 3,5,5-trimethylpentyl, n-dodecyl, n-decyl, cyclopentyl, cyclohexyl, 4-methylcyclohexyl, 3,5-dimethylcyclohexyl, or cycloheptyl radical. In some embodiments, each of R⁴ and R⁵ is hydrogen. In some embodiments, the compound is clofazimine, 2-(p-chlor-anilino)-3-cyclohexylimino-5-(p-chlorophenyl)-3,5-dihydro-phenazine, 2-anilino-3-cyclohexylimino-5-phenyl-3,5-dihydrophenazine. In some embodiments, the compound or the pharmaceutically acceptable salt thereof is clofazimine. In some embodiments, the compound or the pharmaceutically acceptable salt thereof is present in an amount of up to about 1.5% of the total weight of the composition. In some embodiments, the compound or the pharmaceutically acceptable salt thereof is present in an amount of up to about 1% of the total weight of the composition. In some embodiments, the compound or the pharmaceutically acceptable salt thereof is present in an amount of from about 0.001% to about 1% of the total weight of the pharmaceutical composition. In some embodiments, the compound or the pharmaceutically acceptable salt thereof is present in an amount of from about 0.01% to about 1% of the total weight of the pharmaceutical composition. In some embodiments, the compound or the pharmaceutically acceptable salt thereof is present in an amount of from about 0.01% to about 0.1% of the total weight of the pharmaceutical composition. In some embodiments, the compound or the pharmaceutically acceptable salt thereof is present in an amount of from about 0.1% to about 0.5% of the total weight of the pharmaceutical composition. In some embodiments, the pharmaceutical composition is non-aqueous. In some embodiments, the at least one solubilizer comprises one or more of the following: (i) saturated hydrocarbons or their mixtures, (ii) a fatty alcohol having at least nine carbons; and (iii) a fatty acid having at least nine carbons. In some embodiments, the at least one solubilizer comprises saturated hydrocarbons or mixtures thereof. In some embodiments, the at least one solubilizer comprises one or more of the following: (i) petroleum jelly, (ii) one or more alkanes each independently having at least nine carbons, (iii) a fatty alcohol having at least nine carbons; and (iv) a fatty acid having at least nine carbons. In some embodiments, the at least one solubilizer comprises: (i) petroleum jelly, (ii) one or more alkanes each independently having at least nine carbons, and (iii) a fatty alcohol having at least nine carbons. In some embodiments, the solubilizer is present in an amount of from about 50% to about 99% of the total weight of the pharmaceutical composition. In some embodiments, the solubilizer is present in an amount of from about 80% to about 99% of the total weight of the pharmaceutical composition. In some embodiments, the solubilizer is present in an amount of from about 80% to about 99.9% of the total weight of the pharmaceutical composition. In some embodiments, the at least one solubilizer comprises petroleum jelly. In some embodiments, the petroleum jelly is present in an amount of from about 20% to about 99% of the total weight of the pharmaceutical composition. In some embodiments, the petroleum jelly is present in an amount of from about 20% to about 99.9% of the total weight of the pharmaceutical composition. In some embodiments, the petroleum jelly is present in an amount of from about 70% to about 95% of the total weight of the pharmaceutical composition. In some embodiments, the petroleum jelly is present in an amount of from about 80% to about 90% of the total weight of the pharmaceutical composition. In some embodiments, the petroleum jelly is present in an amount of about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 99.9% of the total weight of the pharmaceutical composition. In some embodiments, the at least one solubilizer comprises one or more alkanes each independently having at least nine carbons. In some embodiments, the one or more alkanes comprise nonane, decane, undecane, dodecane, tridecane, tetradecane, pentadecane, hexadecane, heptadecane, octadecane, nonadecane, icosane, heneicosane, docosane, tricosane, tetracosane or a combination thereof, wherein each of the nonane, decane, undecane, dodecane, tridecane, tetradecane, pentadecane, hexadecane, heptadecane, octadecane, nonadecane, icosane, heneicosane, docosane, tricosane, and tetracosane is independently linear or branched. In some embodiments, the one or more alkanes comprise nonane, decane, undecane, dodecane, tridecane, tetradecane, pentadecane, hexadecane, or a combination thereof, wherein each of the nonane, decane, undecane, dodecane, tridecane, tetradecane, pentadecane, and hexadecane is independently linear or branched. In some embodiments, the one or more alkanes are liquid paraffin. In some embodiments, the one or more alkanes are present in an amount of from about 0.5% to about 30% of the total weight of the pharmaceutical composition. In some embodiments, the one or more alkanes are present in an amount of from about 2% to about 15% of the total weight of the pharmaceutical composition. In some embodiments, the one or more alkanes are present in an amount of from about 5% to about 10% of the total weight of the pharmaceutical composition. In some embodiments, the one or more alkanes are present in an amount of about 5%, about 5.5%, about 6%, about 6.5%, about 7%, about 7.5%, about 8%, about 8.5%, about 9%, about 9.5%, or about 10% of the total weight of the pharmaceutical composition. In some embodiments, the one or more alkanes are present in an amount of about 8%, about 8.1%, about 8.2%, about 8.3%, about 8.4%, about 8.5%, about 8.6%, about 8.7%, about 8.8%, about 8.9%, or about 9% of the total weight of the pharmaceutical composition. In some embodiments, the at least one solubilizer comprises a fatty alcohol having at least nine carbons. In some embodiments, the fatty alcohol comprises an alcohol having 9, 10, 11, 12, 13, 14, 15, 16, 17, 18, 19, 20, 21, 22, 23, 24, 25, 26, 27, 28, 29, 30, 31, 32, 33, or 34 carbons. In some embodiments, the fatty alcohol comprises an alcohol having 12 to 24 carbons. In some embodiments, the fatty alcohol is fully saturated or partially saturated. In some embodiments, the fatty alcohol is stearyl alcohol. In some embodiments, the fatty alcohol is present in an amount of from about 0.1% to about 15% of the total weight of the pharmaceutical composition. In some embodiments, the fatty alcohol is present in an amount of from about 1% to about 10% of the total weight of the pharmaceutical composition. In some embodiments, the fatty alcohol is present in an amount of about 2%, about 2.5%, about 3%, about 3.5%, about 4%, about 4.5%, about 5%, about 5.5%, about 6%, about 6.5%, about 7%, about 7.5%, or about 8% of the total weight of the pharmaceutical composition. In some embodiments, the at least one solubilizer comprises a fatty acid having at least nine carbons. In some embodiments, the fatty acid comprises 9, 10, 11, 12, 13, 14, 15, 16, 17, 18, 19, 20, 21, 22, 23, 24, 25, 26, 27, 28, 29, 30, 31, 32, 33, or 34 carbons. In some embodiments, the at least one solubilizer comprises: (i) petroleum jelly in an amount of from about 20% to about 99% of the total weight of the pharmaceutical composition, (ii) liquid paraffin in an amount of from about 0.5% to about 30% of the total weight of the pharmaceutical composition, and (iii) optionally stearyl alcohol in an amount of from about 0.1% to about 15% of the total weight of the pharmaceutical composition. In some embodiments, the at least one solubilizer comprises petroleum jelly in an amount of from about 20% to about 99.9% of the total weight of the pharmaceutical composition. In some embodiments, the at least one solubilizer is petroleum jelly in an amount of from about 20% to about 99.9% of the total weight of the pharmaceutical composition. In some embodiments, the compound is clofazimine, and the at least one solubilizer comprises: (i) petroleum jelly in an amount of from about 70% to about 99% of the total weight of the pharmaceutical composition, and (ii) liquid paraffin in an amount of from about 0.5% to about 30% of the total weight of the pharmaceutical composition. In some embodiments, the compound is clofazimine, and the at least one solubilizer comprises: (i) petroleum jelly in an amount of from about 70% to about 99.9% of the total weight of the pharmaceutical composition, and (ii) optionally liquid paraffin in an amount of from about 0.5% to about 30% of the total weight of the pharmaceutical composition. In some embodiments, the pharmaceutical composition comprises a stratum corneum penetration enhancer. In some embodiments, the stratum corneum penetration enhancer is polyethoxylated sorbitan monooleate, Laurocapram, phospholipids, ethanol, pegylated fatty acid glyceride, or a combination thereof. In some embodiments, the stratum corneum penetration enhancer is lecithin. In some embodiments, the stratum corneum penetration enhancer is present in an amount of from about 1% to about 20% of the total weight of the pharmaceutical composition. In some embodiments, the pharmaceutical composition is formulated as a cream. In some embodiments, the pharmaceutical composition is formulated as a gel. In some embodiments, the formulation described herein is a stable formulation. In some embodiments, the formulation retains about 90 to about 110% w/w of the initial amount of the compound or a pharmaceutically acceptable salt thereof after stored for 6 months at 40° C. and 75% RH. In some embodiments, the formulation retains about 90 to about 110% w/w of the initial amount of the compound or a pharmaceutically acceptable salt thereof after stored for 9 months at 25° C. and 60% RH. In some embodiments, the formulation retains about 90 to about 110% w/w of the initial amount of the compound or a pharmaceutically acceptable salt thereof after stored for 1 month, 2 months, 3 months, 6 months, 9 months, 12 months, 18 months or 24 months at 25° C. or 40° C. In some embodiments, the formulation retains about 95 to about 105% w/w of the initial amount of the compound or a pharmaceutically acceptable salt thereof after stored for 1 month, 2 months, 3 months, 6 months, 9 months, 12 months, 18 months or 24 months at 25° C. or 40° C.

In some embodiments, the pharmaceutical compositions provided herein are gels. In some embodiments, the pharmaceutical compositions provided herein are creams. In some embodiments, the pharmaceutical compositions provided herein are for topical administration.

Further provided herein are pharmaceutical compositions, comprising: an active pharmaceutical ingredient (API) or a pharmaceutically acceptable salt thereof, wherein the active pharmaceutical ingredient has a calculated log P in octanol-water equal or greater than about 4.0; at least one solubilizer; and optionally at least one stratum corneum penetration enhancer, wherein the pharmaceutical composition can be formulated for topical administration. Further provided herein are pharmaceutical compositions wherein the API comprises up to about 10.0% of the total weight of the composition. Further provided herein are pharmaceutical compositions wherein the API comprises up to about 5.0% of the total weight of the composition. Further provided herein are pharmaceutical compositions wherein the API comprises up to about 1.0% of the total weight of the composition. Further provided herein are pharmaceutical compositions wherein the API comprises up to about 0.75% of the total weight of the composition. Further provided herein are pharmaceutical compositions wherein the API comprises up to about 0.5% of the total weight of the composition. Further provided herein are pharmaceutical compositions wherein the API comprises up to about 0.25% of the total weight of the composition. Further provided herein are pharmaceutical compositions wherein the API comprises up to about 0.1% of the total weight of the composition. Further provided herein are pharmaceutical compositions wherein the API comprises from about 0.001% to about 10.0% of the total weight of the composition. Further provided herein are pharmaceutical compositions wherein the API comprises from about 0.01% to about 5.0% of the total weight of the composition. Further provided herein are pharmaceutical compositions wherein the API comprises from about 0.01% to about 2.5% of the total weight of the composition. Further provided herein are pharmaceutical compositions wherein the API comprises from about 0.01% to about 1.0% of the total weight of the composition. Further provided herein are pharmaceutical compositions wherein the API comprises from about 0.01% to about 0.1% of the total weight of the composition. Further provided herein are pharmaceutical compositions wherein the API comprises from about 0.05% to about 1.0% of the total weight of the composition. Further provided herein are pharmaceutical compositions wherein the API comprises from about 0.1% to about 0.5% of the total weight of the composition. Further provided herein are pharmaceutical compositions wherein the log P of the API is greater than about 3.0. Further provided herein are pharmaceutical compositions wherein the log P of the API is greater than about 3.5. Further provided herein are pharmaceutical compositions wherein the log P of the API is greater than about 4.0. Further provided herein are pharmaceutical compositions wherein the log P of the API is greater than about 4.5. Further provided herein are pharmaceutical compositions wherein the log P of the API is greater than about 5.5. Further provided herein are pharmaceutical compositions wherein the log P of the API is greater than about 6.0. Further provided herein are pharmaceutical compositions wherein the log P of the API is greater than about 6.5. Further provided herein are pharmaceutical compositions wherein the log P of the API is greater than about 7.0. Further provided herein are pharmaceutical compositions wherein the log P of the API is greater than about 7.5. Further provided herein are pharmaceutical compositions wherein the log P of the API is from about 4.0 to about 10.0. Further provided herein are pharmaceutical compositions wherein the log P of the API is from about 5.0 to about 9.0. Further provided herein are pharmaceutical compositions wherein the log P of the API is from about 6.0 to about 8.5. Further provided herein are pharmaceutical compositions wherein the log P of the API is from about 6.5 to about 8.0. Further provided herein are pharmaceutical compositions wherein the log P of the API is from about 7.0 to about 8.0. In some embodiments, the API is Hesperetin, Lormetazepam, Naringenin, Cinchonidine, Alprenolol, Propranolol, Ketoprofen, Clofibric acid, Naproxen, Warfarin, Apigenin, Diazepam, Quinine, Quetiapine, Rosiglitazone, Clotiazepam, Tramadol, Fenbufen, Chlorphenamine, Pyrilamine, Venlafaxine, Brompheniramine, Diphenhydramine, Chrysin, Valsartan, Penbutolol, Diltiazem, Ibuprofen, Bupivacaine, Flurbiprofen, Progesterone, Chlordiazepoxide, Trazodone, Haloperidol, Glimepiride, Abiraterone, Indometacin, Clopidogrel, Flurazepam, Duloxetine, Nortriptyline, Celecoxib, Nilotinib, Atorvastatin, Maprotiline, Fluoxetine, Diclofenac, Amitriptyline, Imipramine, Loratadine, Cyproheptadine, Chlorpromazine, Sertraline, Flufenamic acid, Miconazole or Rimonabant. Further provided herein are pharmaceutical compositions wherein the solubilizer is PEG 400, alcohols, polyoxyethylene sorbitan monooleate, polyethers, ethers, glycol ethers, octyl/decyl mono and diglycerides, pegylated fatty acid glycerides, fatty acids, fatty acid esters, or any combination thereof. Further provided herein are pharmaceutical compositions wherein the solubilizer is one or more glycol ethers. Further provided herein are pharmaceutical compositions wherein the solubilizer is a glycol ether. Further provided herein are pharmaceutical compositions wherein the solubilizer is 2-(2-ethoxyethoxy)ethanol. Further provided herein are pharmaceutical compositions wherein the solubilizer is transcutol. Further provided herein are pharmaceutical compositions wherein the stratum corneum penetration enhancer is lecithin, ethanol, octanol, oleic acid, SDS, DMSO, polyoxyethylene sorbitan monooleate (e.g., sold under the trademark Tween®) such as polyoxyethylene (20) sorbitan monooleate and polyoxyethylene (80) sorbitan monooleate, Laurocapram, phospholipids, pegylated fatty acid glyceride, or a combination thereof. Further provided herein are pharmaceutical compositions wherein the stratum corneum penetration enhancer is a phospholipid. Further provided herein are pharmaceutical compositions wherein the stratum corneum penetration enhancer is lecithin. Further provided herein are pharmaceutical compositions wherein the stratum corneum penetration enhancer is lecithin isolated from egg yolk. Further provided herein are pharmaceutical compositions wherein the stratum corneum penetration enhancer is lecithin isolated from soybean. Further provided herein are pharmaceutical compositions wherein the stratum corneum penetration enhancer comprises phosphatidylcholine.

Further provided herein are pharmaceutical compositions, comprising: clofazimine, or a pharmaceutically acceptable salt thereof; at least one solubilizer; and optionally at least one stratum corneum penetration enhancer, wherein the pharmaceutical composition can be formulated for topical administration. Further provided herein are pharmaceutical compositions, wherein the clofazimine, or a pharmaceutically acceptable salt thereof, comprises up to about 5.0% of the total weight of the composition. Further provided herein are pharmaceutical compositions, wherein the clofazimine, or a pharmaceutically acceptable salt thereof, comprises up to about 1.0% of the total weight of the composition. Further provided herein are pharmaceutical compositions, wherein the clofazimine, or a pharmaceutically acceptable salt thereof, comprises up to about 0.75% of the total weight of the composition. Further provided herein are pharmaceutical compositions, wherein the clofazimine, or a pharmaceutically acceptable salt thereof, comprises up to about 0.5% of the total weight of the composition. Further provided herein are pharmaceutical compositions, wherein the clofazimine, or a pharmaceutically acceptable salt thereof, comprises up to about 0.25% of the total weight of the composition. Further provided herein are pharmaceutical compositions, wherein the clofazimine, or a pharmaceutically acceptable salt thereof, comprises up to about 0.1% of the total weight of the composition. Further provided herein are pharmaceutical compositions, wherein the clofazimine, or a pharmaceutically acceptable salt thereof, comprises about 0.01% to about 5.0% of the total weight of the composition. Further provided herein are pharmaceutical compositions, wherein the clofazimine, or a pharmaceutically acceptable salt thereof, comprises about 0.01% to about 2.5% of the total weight of the composition. Further provided herein are pharmaceutical compositions, wherein the clofazimine, or a pharmaceutically acceptable salt thereof, comprises about 0.01% to about 0.1% of the total weight of the composition. Further provided herein are pharmaceutical compositions, wherein the clofazimine, or a pharmaceutically acceptable salt thereof, comprises about 0.01% to about 1.0% of the total weight of the composition. Further provided herein are pharmaceutical compositions, wherein the clofazimine, or a pharmaceutically acceptable salt thereof, comprises about 0.05% to about 1.0% of the total weight of the composition. Further provided herein are pharmaceutical compositions, wherein the clofazimine, or a pharmaceutically acceptable salt thereof, comprises about 0.1% to about 0.5% of the total weight of the composition. Further provided herein are pharmaceutical compositions, wherein the solubilizer comprises up to about 60% of the total weight of the composition. Further provided herein are pharmaceutical compositions, wherein the solubilizer comprises up to about 50% of the total weight of the composition. Further provided herein are pharmaceutical compositions, wherein the solubilizer comprises up to about 40% of the total weight of the composition. Further provided herein are pharmaceutical compositions, wherein the solubilizer comprises up to about 35% of the total weight of the composition. Further provided herein are pharmaceutical compositions, wherein the solubilizer comprises up to about 30% of the total weight of the composition. Further provided herein are pharmaceutical compositions, wherein the solubilizer comprises up to about 25% of the total weight of the composition. Further provided herein are pharmaceutical compositions, wherein the solubilizer comprises up to about 20% of the total weight of the composition. Further provided herein are pharmaceutical compositions, wherein the solubilizer comprises from about 1% to about 60% of the total weight of the composition. Further provided herein are pharmaceutical compositions, wherein the solubilizer comprises from about 5% to about 50% of the total weight of the composition. Further provided herein are pharmaceutical compositions, wherein the solubilizer comprises from about 5% to about 35% of the total weight of the composition. Further provided herein are pharmaceutical compositions, wherein the solubilizer comprises from about 10% to about 40% of the total weight of the composition. Further provided herein are pharmaceutical compositions, wherein the solubilizer comprises from about 10% to about 30% of the total weight of the composition. Further provided herein are pharmaceutical compositions, wherein the solubilizer comprises from about 15% to about 25% of the total weight of the composition. Further provided herein are pharmaceutical compositions, wherein the solubilizer is PEG 400, alcohols, polyoxyethylene sorbitan monooleate, ethers, polyethers, glycol ethers, octyl mono diglycerides, decyl mono diglycerides, octyl/decyl diglycerides, pegylated fatty acid glycerides, fatty acids, fatty acid esters, or any combination thereof. Further provided herein are pharmaceutical compositions, wherein the solubilizer is a glycol ether. Further provided herein are pharmaceutical compositions, wherein the solubilizer is 2-methoxyethanol, 2-ethoxyethanol, 2-propoxyethanol, 2-isopropoxyethanol, 2-butoxyethanol, 2-phenoxyethanol, 2-benzyloxyethanol, 1-methoxy-2-propanol, 2-(2-methoxyethoxy)ethanol, 2-(2-ethoxyethoxy)ethanol, 2-(2-propoxyethoxy)ethanol, 2-(2-butoxyethoxy)ethanol, (2-methoxyethoxy)methanol, (2-ethoxyethoxy)methanol, (2-propoxyethoxy)methanol, or (2-butoxyethoxy)methanol. Further provided herein are pharmaceutical compositions, wherein the solubilizer is 2-(2-methoxyethoxy)ethanol, 2-(2-ethoxyethoxy)ethanol, 2-(2-propoxyethoxy)ethanol, or 2-(2-butoxyethoxy)ethanol. Further provided herein are pharmaceutical compositions, wherein the solubilizer is 2-(2-methoxyethoxy)ethanol. Further provided herein are pharmaceutical compositions, wherein the solubilizer is 2-(2-ethoxyethoxy)ethanol. Further provided herein are pharmaceutical compositions, wherein the solubilizer is 2-(2-propoxyethoxy)ethanol. Further provided herein are pharmaceutical compositions, wherein the solubilizer is 2-(2-butoxyethoxy)ethanol. Further provided herein are pharmaceutical compositions, wherein the solubilizer is dimethoxyethane, diethoxyethane, dipropoxyethane, or dibutoxyethane. Further provided herein are pharmaceutical compositions, wherein the solubilizer is 2-methoxyethyl acetate, 2-ethoxyethyl acetate, 2-butoxyethyl acetate, or 1-methoxy propanol acetate. Further provided herein are pharmaceutical compositions, wherein the stratum corneum penetration enhancer comprises up to about 30% of the total weight of the composition. Further provided herein are pharmaceutical compositions, wherein the stratum corneum penetration enhancer comprises up to about 20% of the total weight of the composition. Further provided herein are pharmaceutical compositions, wherein the stratum corneum penetration enhancer comprises up to about 15% of the total weight of the composition. Further provided herein are pharmaceutical compositions, wherein the stratum corneum penetration enhancer comprises up to about 10% of the total weight of the composition. Further provided herein are pharmaceutical compositions, wherein the stratum corneum penetration enhancer comprises up to about 5% of the total weight of the composition. Further provided herein are pharmaceutical compositions, wherein the stratum corneum penetration enhancer comprises up to about 4% of the total weight of the composition. Further provided herein are pharmaceutical compositions, wherein the stratum corneum penetration enhancer comprises up to about 3% of the total weight of the composition. Further provided herein are pharmaceutical compositions, wherein the stratum corneum penetration enhancer comprises up to about 2% of the total weight of the composition. Further provided herein are pharmaceutical compositions, wherein the stratum corneum penetration enhancer comprises up to about 1% of the total weight of the composition. Further provided herein are pharmaceutical compositions, wherein the stratum corneum penetration enhancer comprises from about 1% to about 30% of the total weight of the composition. Further provided herein are pharmaceutical compositions, wherein the stratum corneum penetration enhancer comprises from about 1% to about 20% of the total weight of the composition. Further provided herein are pharmaceutical compositions, wherein the stratum corneum penetration enhancer comprises from about 2% to about 19% of the total weight of the composition. Further provided herein are pharmaceutical compositions, wherein the stratum corneum penetration enhancer comprises from about 1% to about 15% of the total weight of the composition. Further provided herein are pharmaceutical compositions, wherein the stratum corneum penetration enhancer comprises from about 1% to about 10% of the total weight of the composition. Further provided herein are pharmaceutical compositions, wherein the stratum corneum penetration enhancer comprises from about 2% to about 8% of the total weight of the composition. Further provided herein are pharmaceutical compositions, wherein the stratum corneum penetration enhancer comprises from about 3% to about 7% of the total weight of the composition. Further provided herein are pharmaceutical compositions, wherein the stratum corneum penetration enhancer comprises from about 4% to about 6% of the total weight of the composition. Further provided herein are pharmaceutical compositions, wherein the stratum corneum penetration enhancer is polyoxyethylene sorbitan monooleate (e.g., sold under the trademark Tween®), Laurocapram, phospholipids, ethanol, pegylated fatty acid glyceride, or a combination thereof. Further provided herein are pharmaceutical compositions, wherein the stratum corneum penetration enhancer is a phospholipid. Further provided herein are pharmaceutical compositions, wherein the stratum corneum penetration enhancer is a phosphatidylcholine, a phosphatidylethanolamine, a phosphatidylinositol, a phosphatidylserine, a plasmalogen, a sphingomyelins, lecithin or phosphatidic acid. Further provided herein are pharmaceutical compositions, wherein the stratum corneum penetration enhancer is lecithin. Further provided herein are pharmaceutical compositions, wherein the stratum corneum penetration enhancer is lecithin isolated from egg yolk. Further provided herein are pharmaceutical compositions, wherein the stratum corneum penetration enhancer is lecithin isolated from soybean.

Further provided herein are pharmaceutical compositions, comprising: from about 0.001% to about 2% by weight of clofazimine, or a pharmaceutically acceptable salt thereof from about 5 to about 50% by weight of a solubilizer; and from about 1 to about 20% by weight of a stratum corneum penetration enhancer. Further provided herein are pharmaceutical compositions, comprising: from about 0.01% to about 2% by weight of clofazimine, or a pharmaceutically acceptable salt thereof; from about 5% to about 50% by weight of a solubilizer; and from about 1% to about 20% by weight of a stratum corneum penetration enhancer. Further provided herein are pharmaceutical compositions, comprising: clofazimine or a pharmaceutically acceptable salt thereof, present in an amount of from about 0.01% to about 2.0% of the total weight of the composition; 2-(2-ethoxyethoxy)ethanol, present in an amount of from about 10% to about 30% of the total weight of the composition; and lecithin, present in an amount of from about 2% to about 19% of the total weight of the composition. Further provided herein are pharmaceutical compositions, comprising: clofazimine or a pharmaceutically acceptable salt thereof, present in an amount of from about 0.1% to about 0.5% of the total weight of the composition; 2-(2-ethoxyethoxy)ethanol, present in an amount of from about 15% to about 25% of the total weight of the composition; and lecithin, present in an amount of from about 4% to about 6% of the total weight of the composition. Further provided herein are pharmaceutical compositions, comprising: clofazimine or a pharmaceutically acceptable salt thereof, present in an amount of about 0.1% of the total weight of the composition; 2-(2-ethoxyethoxy)ethanol, present in an amount of about 20% of the total weight of the composition; and lecithin, present in an amount of about 5% of the total weight of the composition. Further provided herein are pharmaceutical compositions, comprising: an active pharmaceutical ingredient or a pharmaceutically acceptable salt thereof, wherein the active pharmaceutical ingredient has a calculated log P in octanol-water equal or greater than 4.0; 2-(2-ethoxyethoxy)ethanol; and lecithin, wherein the pharmaceutical composition can be formulated for topical administration. Further provided herein are pharmaceutical compositions, wherein the API comprises up to about 10.0% of the total weight of the composition. Further provided herein are pharmaceutical compositions, wherein the API comprises up to about 5.0% of the total weight of the composition. Further provided herein are pharmaceutical compositions, wherein the API comprises up to about 1.0% of the total weight of the composition. Further provided herein are pharmaceutical compositions, wherein the API comprises up to about 0.75% of the total weight of the composition. Further provided herein are pharmaceutical compositions, wherein the API comprises up to about 0.5% of the total weight of the composition. Further provided herein are pharmaceutical compositions, wherein the API comprises up to about 0.25% of the total weight of the composition. Further provided herein are pharmaceutical compositions, wherein the API comprises up to about 0.1% of the total weight of the composition. Further provided herein are pharmaceutical compositions, wherein the API comprises from about 0.01% to about 10.0% of the total weight of the composition. Further provided herein are pharmaceutical compositions, wherein the API comprises from about 0.01% to about 5.0% of the total weight of the composition. Further provided herein are pharmaceutical compositions, wherein the API comprises from about 0.01% to about 2.5% of the total weight of the composition. Further provided herein are pharmaceutical compositions, wherein the API comprises from about 0.01% to about 1.0% of the total weight of the composition. Further provided herein are pharmaceutical compositions, wherein the API comprises from about 0.05% to about 1.0% of the total weight of the composition. Further provided herein are pharmaceutical compositions, wherein the API comprises from about 0.1% to about 0.5% of the total weight of the composition. Further provided herein are pharmaceutical compositions, wherein the log P of the API is greater than about 2.5. Further provided herein are pharmaceutical compositions, wherein the log P of the API is greater than about 3.0. Further provided herein are pharmaceutical compositions, wherein the log P of the API is greater than about 3.5. Further provided herein are pharmaceutical compositions, wherein the log P of the API is greater than about 4.0. Further provided herein are pharmaceutical compositions, wherein the log P of the API is greater than about 4.5. Further provided herein are pharmaceutical compositions, wherein the log P of the API is greater than about 5.5. Further provided herein are pharmaceutical compositions, wherein the log P of the API is greater than about 6.0. Further provided herein are pharmaceutical compositions, wherein the log P of the API is greater than about 6.5. Further provided herein are pharmaceutical compositions, wherein the log P of the API is greater than about 7.0. Further provided herein are pharmaceutical compositions, wherein the log P of the API is greater than about 7.5. Further provided herein are pharmaceutical compositions, wherein the log P of the API is from about 4.0 to about 10.0. Further provided herein are pharmaceutical compositions, wherein the log P of the API is from about 5.0 to about 9.0. Further provided herein are pharmaceutical compositions, wherein the log P of the API is from about 6.0 to about 8.5. Further provided herein are pharmaceutical compositions, wherein the log P of the API is from about 6.5 to about 8.0. Further provided herein are pharmaceutical compositions, wherein the log P of the API is from about 7.0 to about 8.0.

Further provided herein are pharmaceutical compositions, comprising: an API or a pharmaceutically acceptable salt thereof, present in an amount of from about 0.01% to about 2.0% of the total weight of the composition; a solubilizer, present in an amount of from about 5% to about 50% of the total weight of the composition; and a stratum corneum penetration enhancer, present in an amount of from about 1% to about 20% of the total weight of the composition. Further provided herein are pharmaceutical compositions, comprising an API or a pharmaceutically acceptable salt thereof, present in an amount of from about 0.01% to about 2.0% of the total weight of the composition; 2-(2-ethoxyethoxy)ethanol, present in an amount of from about 10% to about 30% of the total weight of the composition; and lecithin, present in an amount of from about 2% to about 19% of the total weight of the composition. Further provided herein are pharmaceutical compositions, comprising an API or a pharmaceutically acceptable salt thereof, present in an amount of from about 0.1% to about 0.5% of the total weight of the composition; 2-(2-ethoxyethoxy)ethanol, present in an amount of from about 15% to about 25% of the total weight of the composition; and lecithin, present in an amount of from about 4% to about 6% of the total weight of the composition.

In some embodiments, a formulation described herein is a stable formulation. In some embodiments, the formulation retains about 90 to about 110% w/w of the initial amount of the compound or a pharmaceutically acceptable salt thereof after stored for 6 months at 40° C. and 75% RH. In some embodiments, a total impurity amount in the formulation is no more than 2% w/w based on the initial amount of the compound or a pharmaceutically acceptable salt thereof after stored for 6 months at 40° C. and 75% RH. In some embodiments, an assay value of the formulation is 90% to 110% w/w based on an initial API amount when stored at about 40° C. and 75% RH for 6 months. In some embodiments, an assay value of the formulation is 90% to 110% w/w based on an initial API amount when stored at about 25° C. and 60% RH for 9 months. In some embodiments, a total impurity amount of the formulation is no more than about 2% w/w based on an initial API amount when stored at about 40° C. and 75% RH for 6 months. In some embodiments, a total impurity amount of the formulation is no more than about 2% w/w based on an initial API amount when stored at about 25° C. and 60% RH for 9 months. In some embodiments, the composition of a formulation described herein degrades by less than 2% when held at 60° C. for 3 days. In some embodiments, the composition of a formulation described herein degrades by less than 1% when held at 60° C. for 3 days. In some embodiments, the composition of a formulation described herein degrades by less than 0.5% when held at 60° C. for 3 days. In some embodiments, the composition of a formulation described herein degrades by less than 1% when held at 23° C. for 9 months. In some embodiments, the composition of a formulation described herein degrades by less than 0.5% when held at 23° C. for 9 months. In some embodiments, the composition of a formulation described herein degrades by less than 0.2% when held at 23° C. for 9 months.

Further provided herein are pharmaceutical compositions, further comprising a preservative. In some embodiments, the preservative is calcium benzoate, chlorobutanol, nipalgin or sorbate. In some embodiments, the preservative is calcium benzoate. In some embodiments, the preservative is chlorobutanol. In some embodiments, the preservative is nipalgin. In some embodiments, the preservative is sorbate.

Further provided herein are methods for treating skin diseases. Further provided herein are methods for treating skin diseases, comprising administering the pharmaceutical compositions described herein. Further provided herein are use of the described pharmaceutical compositions in preparation of a medicament for treating a skin disease. In some embodiments, the skin disease is caused by inflammation. In some embodiments, the skin disease is caused by immune inflammation. In some embodiments, the skin disease is psoriasis, vitiligo, lupus erythematosus, chronic eczema, dermatitis or contact dermatitis. In some embodiments, the skin disease is psoriasis. In some embodiments, the skin disease is vitiligo. In some embodiments, the skin disease is lupus. In some embodiments, the skin disease is erythematosus. In some embodiments, the skin disease is eczema or chronic eczema. In some embodiments, the skin disease is dermatitis or contact dermatitis.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a bar graph showing the effects of various solvents (PEG 400, Transcutol, Tween 20, Tween 80, Laurocapram, oleic acid, ethanol and KHS-15) on percutaneous penetration of clofazimine. The X axis indicates the identity of the solvent and the Y axis indicates clofazimine content in μg/cm².

FIG. 2 is a bar graph showing the effects of various solvents (PEG 400, transcutol, Tween 80 and labrasol) on cutaneous penetration of clofazimine. The X axis indicates the identity of the solvent and the Y axis indicates clofazimine content in μg/cm².

FIG. 3 is a bar graph showing the effects of various solvents (PEG 400, transcutol, Tween 80 and labrasol), on cutaneous penetration of clofazimine in gel and solution matrices. The X axis indicates the identity of the solvent and the Y axis indicates clofazimine content in μg/cm².

FIG. 4 is a bar graph showing the effects of various penetration enhancers on cutaneous penetration of clofazimine. The X axis indicates the identity of the penetration enhancer and the Y axis indicates clofazimine content in μg/cm².

FIG. 5 is a bar graph showing the effects of lecithin concentration on cutaneous penetration of clofazimine. The X axis indicates percent lecithin concentration and the Y axis indicates clofazimine content in μg/cm².

FIG. 6 is a bar graph showing the effects of pH on cutaneous penetration of clofazimine in gel preparations. The X axis indicates pH and the Y axis indicates clofazimine content in μg/cm².

FIG. 7 is a bar graph showing the effects of clofazimine concentration on cutaneous penetration of clofazimine. The X axis indicates percent clofazimine concentration and the Y axis indicates clofazimine content in μg/cm².

FIG. 8 is a bar graph showing the effects of gelling agent (carbomer 940) concentration on cutaneous penetration of clofazimine. The X axis indicates percent carbomer 940 concentration and the Y axis indicates clofazimine content in μg/cm².

FIG. 9 is a cartoon representation of the apparatus used to determine the skin permeability of various Clofazimine preparations, as described in Example 4.

FIG. 10 is a bar graph showing that the penetration of clofazimine through the stratum corneum increases with the clofazimine concentration in the topical cream as long as the clofazimine is completely dissolved in the formulation. The X axis indicates the tested formulation, and the Y axis indicates clofazimine content in the inner skin in μg/cm².

FIG. 11 shows the results of a trial comparing the inhibition of IL-2 Secretion of Activated Jurkat Cells treated with clofazimine as described in Example 22.

### DETAILED DESCRIPTION

The present disclosure is generally directed to compositions comprising pharmaceutically active agents that are useful as therapeutics that alleviate, abate or eliminate one or more conditions in a subject in need thereof, as further described herein. In particular, described herein are pharmaceutical compositions, their preparation and use, where the pharmaceutical compositions comprise a lipophilic API, at least one solubilizer, and at least one stratum corneum penetration enhancer in a combination such that the API has improved bioavailability compared to the API alone.

### Definitions

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The singular forms “a,” “an,” and, “the” include plural referents unless the context clearly dictates otherwise. Thus, for example, reference to “the surfactant” includes reference to one or more specific surfactants, reference to “an antioxidant” includes reference to one or more of such additives.

The term “subject” as used herein refers to a mammal (e.g., a human, mouse, rat, guinea pig, dog, cat, horse, cow, pig, or non-human primate, such as a monkey, chimpanzee or baboon).

“Effective amount” and “sufficient amount” may be used interchangeably, and refer to an amount of a substance that is sufficient to achieve an intended purpose or objective.

A “therapeutically effective amount” when used in connection with a pharmaceutical composition described herein is an amount of one or more pharmaceutically active agent(s) sufficient to produce a therapeutic result in a subject in need thereof.

“Therapeutically equivalent” when used in connection with a pharmaceutical composition described herein refers to an amount or quantity of a pharmaceutically acceptable salt or ester of a pharmaceutically active agent that is equivalent to the therapeutically effective amount of the free base or alcohol of the pharmaceutically active agent.

The definitions described herein apply irrespective of whether the terms in question appear alone or in combination. It is contemplated that the definitions described herein can be appended to form chemically-relevant combinations, such as e.g. “heterocycloalkylaryl”, “haloalkylheteroaryl”, “arylalkylheterocycloalkyl”, or “alkoxyalkyl”. The last member of the combination is the radical which is binding to the rest of the molecule. The other members of the combination are attached to the binding radical in reversed order in respect of the literal sequence, e.g. the combination arylalkylheterocycloalkyl refers to a heterocycloalkyl-radical which is substituted by an alkyl which is substituted by an aryl.

The term “substituted,” “substituent” or the like, unless otherwise indicated, can refer to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted.

The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

The term “alkane” refers to a hydrocarbon of formula CₙH₂ₙ₊₂, wherein n is an integer equal to or larger than 1. An alkane can be linear or branched. It is to be understood that isomers of an alkane is encompassed by the term “alkane.” As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl or a lower alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl. The term “perhaloalkyl” denotes an alkyl group where all hydrogen atoms of the alkyl group have been replaced by the same or different halogen atoms.

### Active Pharmaceutical Ingredient (API)

Various embodiments described herein are directed to compositions comprising an effective amount of an active pharmaceutical agent (API). “Active pharmaceutical agent,” “API,” “drug,” “pharmaceutically active agent,” “bioactive agent,” “therapeutic agent,” and “active agent” and the like may be used interchangeably and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, esters, prodrugs, active metabolites, isomers, fragments, analogs, solvates, hydrates, radioisotopes, etc.

The partition-coefficient (P) as referenced herein is a ratio of concentrations of a compound between two immiscible solvent phases at equilibrium. Most commonly, one of the solvents is water and the other is hydrophobic, typically 1-octanol. The logarithm of the ratio is log P, as shown below, (conventionally the lipophilic phase is the numerator and hydrophilic phase is the denominator)

$$log {P}_{octanol / water} = log ( \frac{{[ solute ]}_{octanol}}{{[ solute ]}_{water}} )$$

log P is a measure of lipophilicity or hydrophobicity. Hydrophobicity affects drug absorption, bioavailability, hydrophobic drug-receptor interactions, metabolism of molecules, and toxicity. Hydrophilic compounds are soluble in water (“water-loving”) and polar solvents. Lipophilic compounds are less soluble in water (“water-fearing” or hydrophobic) and polar solvents, but are more soluble in organic solvents. Thus: Low hydrophilicity=high lipophilicity=high log P=poor absorption. High hydrophilicity=low lipophilicity=low log P=good absorption.

Those of skill in the art are familiar with Lapinsky's rule of five, which suggests for compounds to have a reasonable likelihood of being well absorbed their log P must be less than 5.0.

Partition coefficients can be measured experimentally or estimated via calculation. Various methods for calculating (or predicting) log P have been developed, typically by fitting calculated log P values with experimentally measured log P values for training sets of thousands of molecules, mostly drug-like. log P calculations are considered very robust and accurately process many organic molecules. For example, over 50% of molecules log P is predicted with error of less than 0.25, while over 80% with error of less than 0.5. Less than 3.5% of structures are predicted with an error greater 1.0. To distinguish from a measured log P, a calculated log P is sometimes written as clog P.

In some embodiments, the API is lipophilic. In some embodiments, the API is insoluble in polar solvents. In some embodiments, the API is insoluble in aqueous media. In some embodiments, the API is insoluble in water.

In some embodiments, the API has a calculated log P of at least 2.5. In some embodiments, the API has a calculated log P of at least 2.6. In some embodiments, the API has a calculated log P of at least 2.7. In some embodiments, the API has a calculated log P of at least 2.8. In some embodiments, the API has a calculated log P of at least 2.9. In some embodiments, the API has a calculated log P of greater than 3.0. In some embodiments, the API has a calculated log P of greater than 3.1. In some embodiments, the API has a calculated log P of greater than 3.2. In some embodiments, the API has a calculated log P of greater than 3.3. In some embodiments, the API has a calculated log P of greater than 3.4. In some embodiments, the API has a calculated log P of greater than 3.5. In some embodiments, the API has a calculated log P of greater than 3.6. In some embodiments, the API has a calculated log P of greater than 3.7. In some embodiments, the API has a calculated log P of greater than 3.8. In some embodiments, the API has a calculated log P of greater than 3.9. In some embodiments, the API has a calculated log P of greater than 4.0. In some embodiments, the API has a calculated log P of greater than 4.1. In some embodiments, the API has a calculated log P of greater than 4.2. In some embodiments, the API has a calculated log P of greater than 4.3. In some embodiments, the API has a calculated log P of greater than 4.4. In some embodiments, the API has a calculated log P of greater than 4.5. In some embodiments, the API has a calculated log P of greater than 4.6. In some embodiments, the API has a calculated log P of greater than 4.7. In some embodiments, the API has a calculated log P of greater than 4.8. In some embodiments, the API has a calculated log P of greater than 4.9. In some embodiments, the API has a calculated log P of greater than 5.0. In some embodiments, the API has a calculated log P of greater than 5.1. In some embodiments, the API has a calculated log P of greater than 5.2. In some embodiments, the API has a calculated log P of greater than 5.3. In some embodiments, the API has a calculated log P of greater than 5.4. In some embodiments, the API has a calculated log P of greater than 5.5. In some embodiments, the API has a calculated log P of greater than 5.6. In some embodiments, the API has a calculated log P of greater than 5.7. In some embodiments, the API has a calculated log P of greater than 5.8. In some embodiments, the API has a calculated log P of greater than 5.9. In some embodiments, the API has a calculated log P of greater than 6.0. In some embodiments, the API has a calculated log P of greater than 6.1. In some embodiments, the API has a calculated log P of greater than 6.2. In some embodiments, the API has a calculated log P of greater than 6.3. In some embodiments, the API has a calculated log P of greater than 6.4. In some embodiments, the API has a calculated log P of greater than 6.5. In some embodiments, the API has a calculated log P of greater than 6.6. In some embodiments, the API has a calculated log P of greater than 6.7. In some embodiments, the API has a calculated log P of greater than 6.8. In some embodiments, the API has a calculated log P of greater than 6.9. In some embodiments, the API has a calculated log P of greater than 7.0. Exemplary small molecule APIs with calculated log P of greater than 2.0, include, without limitation, those listed in Table 1.

TABLE 1

| API Name | clogP |
|---|---|
| Hesperetin | 2.29 |
| Lormetazepam | 2.4 |
| Naringenin | 2.44 |
| Cinchonidine | 2.49 |
| Alprenolol | 2.65 |
| Propranolol | 2.75 |
| Ketoprofen | 2.76 |
| Clofibric acid | 2.82 |
| Naproxen | 2.82 |
| Warfarin | 2.9 |
| Apigenin | 2.91 |
| Diazepam | 2.96 |
| Quinine | 2.99 |
| Quetiapine | 2.99 |
| Rosiglitazone | 3.02 |
| Clotiazepam | 3.03 |
| Tramadol | 3.1 |
| Fenbufen | 3.14 |
| Chlorphenamine | 3.15 |
| Pyrilamine | 3.23 |
| Venlafaxine | 3.27 |
| Brompheniramine | 3.3 |
| Diphenhydramine | 3.45 |
| Chrysin | 3.56 |
| Valsartan | 3.63 |
| Penbutolol | 3.64 |
| Diltiazem | 3.65 |
| Ibuprofen | 3.68 |
| Bupivacaine | 3.69 |
| Flurbiprofen | 3.75 |
| Progesterone | 3.78 |
| Chlordiazepoxide | 3.79 |
| Trazodone | 3.85 |
| Haloperidol | 3.85 |
| Glimepiride | 3.96 |
| Abiraterone | 4 |
| Indometacin | 4.18 |
| Clopidogrel | 4.21 |
| Flurazepam | 4.22 |
| Duloxetine | 4.26 |
| Nortriptyline | 4.32 |
| Celecoxib | 4.37 |
| Nilotinib | 4.4 |
| Atorvastatin | 4.46 |
| Maprotiline | 4.52 |
| Fluoxetine | 4.57 |
| Diclofenac | 4.73 |
| Amitriptyline | 4.85 |
| Imipramine | 5.04 |
| Loratadine | 5.05 |
| Cyproheptadine | 5.3 |
| Chlorpromazine | 5.3 |
| Sertraline | 5.35 |
| Flufenamic acid | 5.53 |
| Miconazole | 5.81 |
| Rimonabant | 6.47 |
| Clofazimine | 7.7 |

An acid dissociation constant, Ka, (or acidity constant) is a measure of the strength of an acid in solution, typically water. It is the equilibrium constant for the chemical dissociation of acids. In aqueous solution, the equilibrium of acid dissociation is written:

HA+H₂O [symbol] A⁻+H₃O+

where HA is an acid that dissociates into A⁻, (the conjugate base of the acid) and a hydrogen ion (which combines with a water molecule to make a hydronium ion, H₃O⁺). The dissociation constant can also be written with the H₂O removed:

HA [symbol] A⁻+H+

$${K}_{a} = \frac{[ {A}^{-} ] [ {H}^{+} ]}{[ HA ]}$$

pKₐ, the logarithmic value of Kₐ, is more often used to express acid strength/weakness:

pKₐ=−log₁₀(Kₐ)

The more positive the value of pKₐ, the smaller the extent of dissociation, and the weaker the acid. In general:

pKₐ=−2 to 12→weak acid (little or only partial dissociation in water)

pKa<−2→strong acid (completely or mostly dissociated in water)

In some embodiments, an API described herein is a weak base. In some embodiments, the API comprises a weak base functional group. In some embodiments, the API has a pKa of equal or greater than 3.0. In some embodiments, the API has a pKa of equal or greater than 3.5. In some embodiments, the API has a pKa of equal or greater than 4.0. In some embodiments, the API has a pKa of equal or greater than 4.5. In some embodiments, the API has a pKa of equal or greater than 5.0. In some embodiments, the API has a pKa of equal or greater than 5.5. In some embodiments, the API has a pKa of equal or greater than 6.0. In some embodiments, the API has a pKa of equal or greater than 6.5. In some embodiments, the API has a pKa of equal or greater than 7.0. In some embodiments, the API has a pKa of equal or greater than 7.5. In some embodiments, the API has a pKa of equal or greater than 8.0.

In some embodiments, an API described herein API is present in the form of a free base. In some embodiments, the API is present in the form of a pharmaceutically acceptable salt. Pharmaceutically acceptable salts include, but are not limited to, metal salts, such as sodium salts, potassium salts, and lithium salts; alkaline earth metals, such as calcium salts, magnesium salts, and the like; organic amine salts, such as triethylamine salts, pyridine salts, nicotine salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Pharmaceutically acceptable salts further include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt. In some embodiments, the API is clofazimine, or a pharmaceutically acceptable salt thereof. In some embodiments, the API is one listed in Table 1, or a pharmaceutically acceptable salt thereof.

In some embodiments, an API described herein comprises from about 0.0001% to about 50% of the total weight of a herein described pharmaceutical composition. In some embodiments, the API comprises from about 0.001% to about 10% of the total weight of a herein described pharmaceutical composition. In some embodiments, the API comprises from about 0.001%, 0.005%, 0.01%, 0.05%, or 0.1% to about 0.2%, 0.5%, 1%, 2% or 5% of the total weight of a herein described pharmaceutical composition. In some embodiments, the API comprises from about 0.001% to about 2.5% of the total weight of a herein described pharmaceutical composition. In some embodiments, the API comprises from about 0.005% to about 1% of the total weight of a herein described pharmaceutical composition. In some embodiments, the API comprises from about 0.05% to about 1% of the total weight of a herein described pharmaceutical composition. In some embodiments, the API comprises from about 0.01% to about 1% of the total weight of a herein described pharmaceutical composition. In some embodiments, the API comprises from about 0.01% to about 0.5% of the total weight of a herein described pharmaceutical composition. In some embodiments, the API comprises from about 0.01% to about 2% of the total weight of a herein described pharmaceutical composition. In some embodiments, the API comprises from about 0.01% to about 0.05% of the total weight of a herein described pharmaceutical composition. In some embodiments, the API comprises about 0.01% of the total weight of the composition. In some embodiments, the API comprises about 0.02% of the total weight of the composition. In some embodiments, the API comprises about 0.025% of the total weight of the composition. In some embodiments, the API comprises about 0.03% of the total weight of the composition. In some embodiments, the API comprises about 0.04% of the total weight of the composition. In some embodiments, the API comprises about 0.05% of the total weight of the composition. In some embodiments, the API comprises about 0.06% of the total weight of the composition. In some embodiments, the API comprises about 0.07% of the total weight of the composition. In some embodiments, the API comprises about 0.075% of the total weight of the composition. In some embodiments, the API comprises about 0.08% of the total weight of the composition. In some embodiments, the API comprises about 0.09% of the total weight of the composition. In some embodiments, the API comprises about 0.1% of the total weight of the composition. In some embodiments, the API comprises about 0.11% of the total weight of the composition. In some embodiments, the API comprises about 0.12% of the total weight of the composition. In some embodiments, the API comprises about 0.125% of the total weight of the composition. In some embodiments, the API comprises about 0.13% of the total weight of the composition. In some embodiments, the API comprises about 0.14% of the total weight of the composition. In some embodiments, the API comprises about 0.15% of the total weight of the composition. In some embodiments, the API comprises about 0.16% of the total weight of the composition. In some embodiments, the API comprises about 0.17% of the total weight of the composition. In some embodiments, the API comprises about 0.175% of the total weight of the composition. In some embodiments, the API comprises about 0.18% of the total weight of the composition. In some embodiments, the API comprises about 0.19% of the total weight of the composition. In some embodiments, the API comprises about 0.20% of the total weight of the composition. In some embodiments, the API comprises about 0.25% of the total weight of the composition. In some embodiments, the API comprises about 0.3% of the total weight of the composition. In some embodiments, the API comprises about 0.35% of the total weight of the composition. In some embodiments, the API comprises about 0.4% of the total weight of the composition. In some embodiments, the API comprises about 0.45% of the total weight of the composition. In some embodiments, the API comprises about 0.5% of the total weight of the composition. In some embodiments, the API comprises about 0.6% of the total weight of the composition. In some embodiments, the API comprises about 0.7% of the total weight of the composition. In some embodiments, the API comprises about 0.8% of the total weight of the composition. In some embodiments, the API comprises about 0.9% of the total weight of the composition. In some embodiments, the API comprises about 1% of the total weight of the composition. In some embodiments, the API comprises about 1.1% of the total weight of the composition. In some embodiments, the API comprises about 1.2% of the total weight of the composition. In some embodiments, the API comprises about 1.3% of the total weight of the composition. In some embodiments, the API comprises about 1.4% of the total weight of the composition. In some embodiments, the API comprises about 1.5% of the total weight of the composition. In some embodiments, the API comprises about 1.6% of the total weight of the composition. In some embodiments, the API comprises about 1.7% of the total weight of the composition. In some embodiments, the API comprises about 1.8% of the total weight of the composition. In some embodiments, the API comprises about 1.9% of the total weight of the composition. In some embodiments, the API comprises about 2% of the total weight of the composition. In some embodiments, the API is clofazimine, or a pharmaceutically acceptable salt thereof. In some embodiments, the API is one listed in Table 1, or a pharmaceutically acceptable salt thereof. In some embodiments, the API is clofazimine or a pharmaceutically acceptable salt thereof.

In some embodiments, pharmaceutical compositions described herein comprise a lipophilic API, a solubilizer and optionally a penetration enhancer. In some embodiments, the pharmaceutical compositions described herein comprise a lipophilic API with a clog P greater than 4, a solubilizer with solubility of APIs with a clog P greater than 4 and optionally a penetration enhancer. In some embodiments, the solubility of the API in the solubilizer is at least 0.1 mg/mL. In some embodiments, the solubility of the API in the solubilizer is at least 0.5 mg/mL. In some embodiments, the solubility of the API in the solubilizer is at least 3 mg/mL. In some embodiments, the solubility of the API in the solubilizer is at least 4 mg/mL. In some embodiments, the solubility of the API in the solubilizer is at least 5 mg/mL. In some embodiments, the solubility of the API in the solubilizer is at least 6 mg/mL. In some embodiments, the solubility of the API in the solubilizer is at least 7 mg/mL. In some embodiments, the solubility of the API in the solubilizer is at least 8 mg/mL. In some embodiments, the solubility of the API in the solubilizer is at least 9 mg/mL. In some embodiments, the solubility of the API in the solubilizer is at least 10 mg/mL. In some embodiments, the solubility of the API in the solubilizer is at least 11 mg/mL. In some embodiments, the solubility of the API in the solubilizer is at least 12 mg/mL. In some embodiments, the solubility of the API in the solubilizer is at least 13 mg/mL. In some embodiments, the solubility of the API in the solubilizer is at least 14 mg/mL. In some embodiments, the solubility of the API in the solubilizer is at least 15 mg/mL. In some embodiments, the solubility of the API in the solubilizer is at least 20 mg/mL. In some embodiments, the solubility of the API in the solubilizer is at least 25 mg/mL. In some embodiments, the solubility of the API in the solubilizer is at least 50 mg/mL. In some embodiments, the API is clofazimine, or a pharmaceutically acceptable salt thereof. In some embodiments, the API is one listed in Table 1, or a pharmaceutically acceptable salt thereof.

In some embodiments, described herein is a pharmaceutical composition comprising an active pharmaceutical ingredient (API) or a pharmaceutically acceptable salt thereof, and at least one solubilizer, wherein the compound has a structure of Formula (I):

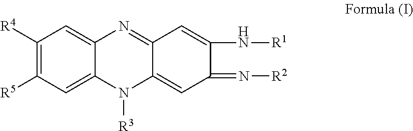

wherein,
each of R¹, R² and R³ is independently an aryl radical selected from the group consisting of phenyl, chlorophenyl, lower alkylphenyl and lower alkoxyphenyl; a C₁-C₁₂ alkyl; a C₃-C₈ cycloalkyl; or a C₁-C₁₁ heteroalkyl, wherein each of the aryl, alkyl, cycloalkyl, and heteroalkyl is substituted or unsubstituted;
R₄ represents a hydrogen or halogen atom; and
R₅ represents a hydrogen or halogen atom.
   In some embodiments, the compound or the pharmaceutically acceptable salt thereof is dissolved in the at least one solubilizer. In some embodiments, the pharmaceutical composition is formulated for topical administration. In some embodiments, R¹ is phenyl, chlorophenyl, lower alkylphenyl, or lower alkoxyphenyl. In some embodiments, R³ is phenyl, chlorophenyl, lower alkylphenyl, or lower alkoxyphenyl. In some embodiments, both R¹ and R³ are selected from phenyl, chlorophenyl, lower alkylphenyl, and lower alkoxyphenyl, wherein the phenyl, chlorophenyl, lower alkylphenyl, and lower alkoxyphenyl is substituted or unsubstituted. In some embodiments, each of R¹ and R³ is independently an ethyl, propyl, butyl, pentyl, hexyl, heptyl, decyl, cyclohexyl, or cycloheptyl radical. In some embodiments, R² is an ethyl, propyl, butyl, pentyl, hexyl, heptyl, decyl, cyclohexyl, or cycloheptyl radical. In some embodiments, R² is an ethyl, n-propyl, 1-methylethyl (i-propyl), n-butyl, heptyl, 1,3-dimethylbutyl, sec-butyl, 1,1-dimethylethyl (t-butyl), 3,5,5-trimethylpentyl, n-dodecyl, n-decyl, cyclopentyl, cyclohexyl, 4-methylcyclohexyl, 3,5-dimethylcyclohexyl, or cycloheptyl radical. In some embodiments, each of R⁴ and R⁵ is hydrogen. In some embodiments, the compound is clofazimine, 2-(p-chlor-anilino)-3-cyclohexylimino-5-(p-chlorophenyl)-3,5-dihydro-phenazine, 2-anilino-3-cyclohexylimino-5-phenyl-3,5-dihydrophenazine. In some embodiments, the compound is clofazimine.

### Solubilizing Agent/Solubilizer/Solvent

In some embodiments, pharmaceutical compositions described herein comprise a lipophilic API, a solubilizer, and optionally a penetration enhancer. Solubilizing agents, or solubilizers or solvents, or other similar terms known to those of skill in the art, refer to an agent or combination of agents that solubilize or partially solubilize an API, as described herein. In some embodiments, the pharmaceutical compositions comprise a lipophilic API with a clog P greater than 4, a solvent with solubility of API with a clog P greater than 4 and a penetration enhancer.

In one aspect, described herein is a topical pharmaceutical composition comprising an active pharmaceutical ingredient (API) (such as clofazimine) or a pharmaceutically acceptable salt thereof and at least one solubilizer. In some embodiments, described herein is a topical pharmaceutical composition, comprising: an API or a pharmaceutically acceptable salt thereof present in an amount of from about 0.001% to about 10% of the total weight of the composition, wherein the active pharmaceutical ingredient has a calculated log P in octanol-water equal or greater than about 4.0; and at least one solubilizer present in an amount of from about 20% to about 99.999% of the total weight of the composition. In some embodiments, the pharmaceutical composition is a non-aqueous composition. In some embodiments, the API or the pharmaceutically acceptable salt thereof is dissolved in the at least one solubilizer. In some embodiments, polarizing microscope can be used to determine whether an API is dissolved in the at least one solubilizer. In some embodiments, the API has a calculated log P in octanol-water equal or greater than about 4.5, 5.0, 5.5, 6.0, 6.5, 7.0, or 7.5. In some embodiments, the API has a calculated log P in octanol-water within a range of from about 4.0, 4.5, 5.0, 5.5 or 6 to about 7, 8, 9, or 10. In some embodiments, the API or the pharmaceutically acceptable salt thereof is present in an amount of from about 0.01% to about 5% of the total weight of the pharmaceutical composition. In some embodiments, the API or the pharmaceutically acceptable salt thereof is present in an amount of from about 0.001% to about 10% of the total weight of the pharmaceutical composition. In some embodiments, the API or the pharmaceutically acceptable salt thereof is present in an amount of from about 0.01% to about 0.1% of the total weight of the pharmaceutical composition. In some embodiments, the API or the pharmaceutically acceptable salt thereof is present in an amount of from about 0.01% to about 2% of the total weight of the pharmaceutical composition. In some embodiments, the API or the pharmaceutically acceptable salt thereof is present in an amount of from about 0.01% to about 1% of the total weight of the pharmaceutical composition. In some embodiments, the API or the pharmaceutically acceptable salt thereof is present in an amount of from about 0.1% to about 0.5% of the total weight of the pharmaceutical composition.

In some embodiments, described herein is a topical pharmaceutical composition, comprising: clofazimine or a pharmaceutically acceptable salt thereof present in an amount of from about 0.001% to about 10% of the total weight of the composition; and at least one solubilizer present in an amount of from about 20% to about 99.999% of the total weight of the composition. In some embodiments, the pharmaceutical composition is a non-aqueous composition. In some embodiments, the clofazimine or the pharmaceutically acceptable salt thereof is dissolved in the at least one solubilizer. In some embodiments, the clofazimine or the pharmaceutically acceptable salt thereof is clofazimine. In some embodiments, the clofazimine or the pharmaceutically acceptable salt thereof is present in an amount of from about 0.01% to about 5% of the total weight of the pharmaceutical composition. In some embodiments, the clofazimine or the pharmaceutically acceptable salt thereof is present in an amount of from about 0.01% to about 2% of the total weight of the pharmaceutical composition. In some embodiments, the clofazimine or the pharmaceutically acceptable salt thereof is present in an amount of from about 0.01% to about 1% of the total weight of the pharmaceutical composition. In some embodiments, the clofazimine or the pharmaceutically acceptable salt thereof is present in an amount of from about 0.1% to about 0.5% of the total weight of the pharmaceutical composition. In some embodiments, the clofazimine or the pharmaceutically acceptable salt thereof is present in an amount of from about 0.01% to about 0.1% of the total weight of the pharmaceutical composition. In some embodiments, the clofazimine or the pharmaceutically acceptable salt thereof is present in an amount of from about 0.01% to about 0.2% of the total weight of the pharmaceutical composition. In some embodiments, the clofazimine or the pharmaceutically acceptable salt thereof is present in an amount of about 0.5% of the total weight of the pharmaceutical composition.

In some embodiments, the solubilizer is an amide, an ester, an alcohol, such as ethanol, isopropanol, butanol, benzyl alcohol, a polyol, polyvinylalcohol, ethylene glycol, propylene glycol, polypropylene glycol, a butanediol, a propylene glycol ether, glycerol, a glyceride, a fatty acid, a fatty acid ester, a fatty acid glyceride, a polyethylene glycol fatty acid ester, a vegetable oil, oleic acid, castor oil, a diethylene glycol ether, a PEGyled glyceride, 2-(2-ethoxyethoxy) ethanol (Transcutol), PEG 400, polyoxyethylene sorbitan monooleate (e.g., those sold under the trademark Tween®), KHS-15, Laurocapram (i.e., N-Dodecylcaprolactan or 1-dodecylazepan-2-one), pentaerythritol, sorbitol, mannitol, dimethyl isosorbide, hydroxypropyl methylcellulose and other cellulose derivatives, maltodextrins, cyclodextrins, cyclodextrin derivatives, isomers thereof or combination thereof.

In some embodiments, the solubilizer is a fatty acid, a glyceride, a vegetable oil, oleic acid, a PEGyled glyceride, 2-(2-ethoxyethoxy) ethanol (Transcutol), PEG 400, polyoxyethylene (20) sorbitan monooleate (e.g., sold under the trademark Tween-20®), polyoxyethylene (80) sorbitan monooleate (e.g., sold under the trademark Tween-80®), ethanol, KHS-15, Laurocapram (i.e., N-Dodecylcaprolactam or 1-dodecylazepan-2-one) (e.g., sold under the trademark Azone), or a combination thereof. In some embodiments, the solubilizer comprises Laurocapram. In some embodiments, the solubilizer comprises ethanol. In some embodiments, the solubilizer comprises Transcutol. In some embodiments, the solubilizer comprises 2-(2-ethoxyethoxy) ethanol. In some embodiments, the API is clofazimine, or a pharmaceutically acceptable salt thereof. In some embodiments, the API is one listed in Table 1, or a pharmaceutically acceptable salt thereof.

In some embodiments, the solubilizer comprises a weight percent of the composition of about 1% to about 50%. In some embodiments, the solubilizer comprises a weight percent of the composition of about 1% to about 3%, about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 3% to about 5%, about 3% to about 10%, about 3% to about 15%, about 3% to about 20%, about 3% to about 25%, about 3% to about 30%, about 3% to about 35%, about 3% to about 40%, about 3% to about 45%, about 3% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the solubilizer comprises a weight percent of the composition of about 1%, about 3%, about 5%, about 10%, about 15%, about 20%, about 25%, about 30%, about 35%, about 40%, about 45%, or about 50%. In some embodiments, the solubilizer comprises a weight percent of the composition of at least about 1%, about 3%, about 5%, about 10%, about 15%, about 20%, about 25%, about 30%, about 35%, about 40%, or about 45%. In some embodiments, the solubilizer comprises a weight percent of the composition of at most about 3%, about 5%, about 10%, about 15%, about 20%, about 25%, about 30%, about 35%, about 40%, about 45%, or about 50%.

In some embodiments, the Transcutol comprises a weight percent of the composition of about 1% to about 50%. In some embodiments, the Transcutol comprises a weight percent of the composition of about 1% to about 3%, about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 3% to about 5%, about 3% to about 10%, about 3% to about 15%, about 3% to about 20%, about 3% to about 25%, about 3% to about 30%, about 3% to about 35%, about 3% to about 40%, about 3% to about 45%, about 3% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the Transcutol comprises a weight percent of the composition of about 1%, about 3%, about 5%, about 10%, about 15%, about 20%, about 25%, about 30%, about 35%, about 40%, about 45%, or about 50%. In some embodiments, the Transcutol comprises a weight percent of the composition of at least about 1%, about 3%, about 5%, about 10%, about 15%, about 20%, about 25%, about 30%, about 35%, about 40%, or about 45%. In some embodiments, the Transcutol comprises a weight percent of the composition of at most about 3%, about 5%, about 10%, about 15%, about 20%, about 25%, about 30%, about 35%, about 40%, about 45%, or about 50%.

In some embodiments, pharmaceutical formulations described herein comprise one or more fatty acids or derivatives thereof. Exemplary fatty acids include carboxylic acids consisting of a hydrocarbon chain and a terminal carboxy (—COOH) group. The hydrocarbon chain may be saturated (containing no carbon-carbon double bonds) or unsaturated, including monounsaturated (containing one carbon-carbon double bond) or polyunsaturated (containing more than one carbon-carbon double bond). Fatty acids can contain carbon chains that are straight (or unbranched), carbon chains, or they can contain branched carbon chains. In some embodiments, the one or more fatty acids comprise C₉-C₂₀ fatty acids, C₉-C₁₂ fatty acids, C₉-C₁₅ fatty acids, C₉-C₁₈ fatty acids, C₁₂-C₁₈ fatty acids, or C₉-C₃₂ fatty acids.

In some embodiments, pharmaceutical formulations described herein comprises one or more long chain fatty acids, or a combination thereof. In some embodiments, the long chain fatty acids are carboxylic acids comprising at least 12 carbon atoms. In some embodiments, the long chain fatty acid comprises at least 11 carbon atoms. In some embodiments, the long chain fatty acid comprises at least 12 carbon atoms. In some embodiments, the long chain fatty acid comprises at least 13 carbon atoms. In some embodiments, the long chain fatty acid comprises at least 14 carbon atoms. In some embodiments, the long chain fatty acid comprises at least 15 carbon atoms. In some embodiments, the long chain fatty acid comprises at least 16 carbon atoms. In some embodiments, the long chain fatty acid comprises at least 17 carbon atoms. In some embodiments, the long chain fatty acid comprises at least 18 carbon atoms. In some embodiments, the long chain fatty acid comprises at least 19 carbon atoms. In some embodiments, the long chain fatty acid comprises at least 20 carbon atoms. Examples of straight chain, saturated fatty acids include, but are not limited to those listed in Table 2. In some embodiments, pharmaceutical formulations described herein comprises one or more fatty acids of Table 2 and Table 3.

TABLE 2

| Common name | Chemical structure |
|---|---|
| Lauric acid | CH₃(CH₂)₁₀COOH |
| Myristic acid | CH₃(CH₂)₁₂COOH |
| Palmitic acid | CH₃(CH₂)₁₄COOH |
| Stearic acid | CH₃(CH₂)₁₆COOH |
| Arachidic acid | CH₃(CH₂)₁₈COOH |
| Behenic acid | CH₃(CH₂)₂₀COOH |
| Lignoceric acid | CH₃(CH₂)₂₂COOH |
| Cerotic acid | CH₃(CH₂)₂₄COOH |

Examples of straight chain, unsaturated fatty acids include, but are not limited to, those listed in Table 3.

TABLE 3

| Name | #C | Structural Formula |
|---|---|---|
| Myristoleic acid | 14 | CH₃(CH2)₃CH═CH(CH₂)₇COOH |
| Palmitoleic acid | 16 | CH₃(CH₂)₅CH═CH(CH₂)₇COOH |
| Sapienic acid | 16 | CH₃(CH₂)₈CH═CH(CH₂)₄COOH |
| Oleic acid | 18 | CH₃(CH₂)₇CH═CH(CH₂)₇COOH |
| Elaidic acid | 18 | CH₃(CH₂)₇CH═CH(CH₂)₇COOH |
| Vaccenic acid | 18 | CH₃(CH₂)₅CH═CH(CH₂)₉COOH |
| Linoleic acid | 18 | CH₃(CH₂)₄CH═CHCH₂CH═CH(CH₂)₇COOH |
| Linolelaidic acid | 18 | CH₃(CH₂)₄CH═CHCH₂CH═CH(CH₂)₇COOH |
| γ-Linolenic acid | 18 | CH₃(CH₂)₄CH═CHCH₂CH═CHCH₂CH═CH(CH₂)₄COOH |
| α-Linolenic acid | 18 | CH₃CH₂CH═CHCH₂CH═CHCH₂CH═CH(CH₂)₇COOH |
| Stearidonic acid | 18 | CH₃CH₂CH═CHCH₂CH═CHCH₂CH═CHCH₂CH═CH(CH₂)₄COOH |
| Paullinic acid | 20 | CH₃(CH₂)₅CH═CH(CH₂)₁₁COOH |
| Gondoic acid | 20 | CH₃(CH₂)₇CH═CH(CH₂)₉COOH |
| Dihomo-γ-linolenic acid | 20 | CH₃(CH₂)₄CH═CHCH₂CH═CHCH₂CH═CH(CH₂)₆COOH |
| Mead acid | 20 | CH₃(CH₂)₇CH═CHCH₂CH═CHCH₂CH═CH(CH₂)₃COOH |
| Arachidonic acid | 20 | CH₃(CH₂)₄CH═CHCH₂CH═CHCH₂CH═CHCH₂CH═CH(CH₂)₃COOH |
| Eicosapentaenoic acid | 20 | CH₃CH₂CH═CHCH₂CH═CHCH₂CH═CHCH₂CH═CHCH₂CH═CH(CH₂)₃COOH |
| Erucic acid | 22 | CH₃(CH₂)₇CH═CH(CH₂)₁₁COOH |
| Docosatetraenoic acid | 22 | CH₃(CH₂)₄CH═CHCH₂CH═CHCH₂CH═CHCH₂CH═CH(CH₂)₅COOH |
| Docosahexaenoic acid | 22 | CH₃CH₂CH═CHCH₂CH═CHCH₂CH═CHCH₂CH═CHCH₂CH═CHCH₂CH═CH(CH₂)₂COOH |
| Nervonic acid | 24 | CH₃(CH₂)₇CH═CH(CH₂)₁₃COOH |

In some embodiments, the solubilizer comprises a glyceride. Glycerides are fatty acid esters of glycerol.

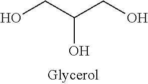

Glycerides may be monoglycerides, diglycerides and triglycerides, wherein glycerol is esterified with one, two, or three fatty acids. Other terms in the art for triglycerides include TG's, triacylglycerols, TAG's, triacylglycerides, fats and the like and are used interchangeably herein. Glycerides may be further modified by substitution onto one or more for the hydrocarbon chains.

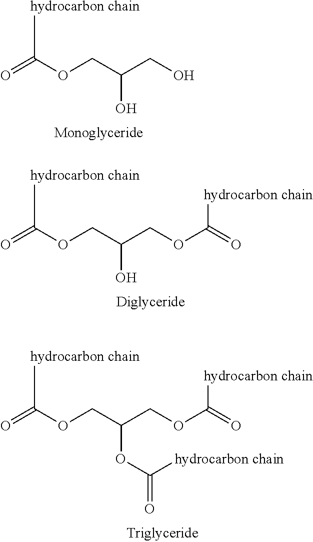

Examples of mono- and diglycerides include, but are not limited to, Monopalmitolein, Monoelaidin, Monocaproin, Monocaprylin, Monocaprin, Monolaurin, Glyceryl monomyristate, Glyceryl monooleate, Glyceryl monooleate, Glycerol monooleate/linoleate, Glycerol monolinoleate, Glyceryl ricinoleate, Glyceryl monolaurate, Glycerol monopalmitate, Glycerol monostearate, Glyceryl mono-, dioleate, Glyceryl palmitic/stearic, Glyceryl acetate, Glyceryl laurate, Glyceryl citrate/lactate/oleate/linoleate, Glyceryl caprylate, Glyceryl caprylate/caprate, Caprylic acid mono, diglycerides, Caprylic/capric glycerides, Mono- and diacetylated monoglycerides, Glyceryl monostearate, Lactic acid esters of mono, diglycerides, Dicaproin, Dicaprin, Dioctanoin, Dimyristin, Dipalmitin, Distearin, Glyceryl dilaurate, Glyceryl dioleate, Glycerol esters of fatty acids, 1,2 and 1,3-diolein, Dielaidin, Dilinolein, and combinations thereof. Other fatty acids include stearyl alcohol, capric acid, caprylic acid, lauric acid, myristic acid, stearic acid, oleic acid, linoleic acid, linolenic acid, arachnidoic acid, behenic acid, and their corresponding pharmaceutically acceptable salts. Preferred fatty acid and fatty alcohol derivatives include sodium dioctyl sulfosuccinate, sodium lauryl sulfate, amide esters (e.g., lauric acid diethanolamide, sodium lauryl sarcosinate, lauroyl carnitine, palmitoyl carnitine and myristoyl carnitine), esters with hydroxy-acids (e.g., sodium stearoyl lactylate); sugar esters [e.g., lauryl lactate, glucose monocaprylate, diglucose monocaprylate, sucrose laurate, sorbitan monolaurate (Arlacel® 20), sorbitan monopalmitate (Span-40), sorbitan monooleate (Span-80), sorbitan monostearate and sorbitan tristearate, lower alcohol fatty acid esters [e.g., ethyl oleate (Crodamol EO), isopropyl myristate (Crodamol IPM) and isopropyl palmitate (Crodamol IPP)], esters with propylene glycol [e.g., propylene glycol monolaurate (Lauroglycol FCC), propylene glycol ricinoleate (Propymuls), propylene glycol monooleate (Myverol® P-06), propylene glycol monocaprylate (Capryol® 90), propylene glycol dicaprylate/dicaprate (Captex® 200) and propylene glycol dioctanoate (Captex 800)], esters with glycerol [e.g., glyceryl monooleate (Peceol), glyceryl ricinoleate, glyceryl laurate, glyceryl dilaurate (Capmul® GDL), glyceryl dioleate (Capmul GDO), glycerol monolinoleate (Maisine®), glyceryl mono/dioleate (Capmul GMO-K), glyceryl caprylate/caprate (Capmul MCM), caprylic acid mono/diglycerides (Imwitor® 988), mono- and diacetylated monoglycerides (Myvacet® 9-45)], triglycerides [e.g., corn oil, almond oil, soybean oil, coconut oil, castor oil, hydrogenated castor oil, hydrogenated coconut oil, Pureco 100, Hydrokote APS, Captex 300, 350, Miglyol 812, Miglyol 818 and Gelucire 33/01)], mixtures of propylene glycol esters and glycerol esters [e.g., mixture of oleic acid esters of propylene glycol and glycerol (Arlacel 186)], and polyglycerized fatty acids such as polyglyceryl oleate (Plurol® Oleique), polyglyceryl-2 dioleate (Nikko] DGDO), polyglyceryl-10 trioleate, polyglyceryl-10 laurate (Nikkol Decaglyn 1-L), polyglyceryl-10 oleate (Nikkol Decaglyn 1-O), and polyglyceryl-10 mono, dioleate (Caprol® PEG 860).

Additional fatty acid derivatives include polyethoxylated fatty acids (e.g., PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate), PEG-fatty acid diesters (e.g., PEG-20 dilaurate, PEG-20 dioleate, PEG-20 distearate, PEG-32 dilaurate and PEG-32 dioleate), PEG-fatty acid mono- and di-ester mixtures, polyethylene glycol glycerol fatty acid esters (e.g., PEG'ylated glycerol 12 acyloxy-stearate, PEG-20 glyceryl laurate, PEG-30 glyceryl laurate, PEG-40 glyceryl laurate, PEG-20 glyceryl oleate and PEG-30 glyceryl oleate) and alcohol-oil transesterification products [e.g., polyoxyl 40 castor oil (Cremophor® RH40), polyoxyl 35 castor oil (Cremophor EL or Incrocas 35), PEG-25 trioleate (TAGAT® TO), PEG-60 corn glycerides (Crovol M70), PEG-60 almond oil (Crovol A70), PEG 40 palm kernel oil (Crovol PK70), PEG-50 castor oil (Emalex C-50), PEG-50 hydrogenated castor oil (Emalex HC-50), PEG-60 hydrogenated castor oil (Cremophor RH60), PEG-8 caprylic/capric glycerides (Labrasol®), lauroyl macrogol 32 glycerides (Gelucire® 44/14), linoleoyl macrogoglycerides (Labrafil®), stearoyl macrogol-32 glycerides (Gelucire 50/13), and PEG-6 caprylic/capric glycerides (Softigen® 767)].

Bile acid and sterol derivatives include, but are not limited to, cholate, ursodeoxycholate, chenodeoxycholate, taurochenodeoxycholate, tauroursodeoxycholate, glycochenodeoxycholate, glycoursodeoxycholate, sterols and sterol esters or ethers such as PEG-24 cholesterol ether.

Tocol derivatives include derivatives of substances with the tocol structure [2 methyl-2-(4,8,12-trimethyltridecyl)chroman-6-ol] or the tocotrienol structure [2 methyl-2-(4,8,12-trimethyltrideca-3,7,11-trienyl)chroman-6-of]. In particular, the mono-, di-, trimethyl-tocols, commonly known as tocopherols and their organic acid esters such as the acetate, nicotinate, succinate, and polyethylnene glycol succinate esters are included. For example, a-tocopherol acetate, a-tocopherol nicotinate, a tocopherol succinate, a.-tocopherol polyethyleneglycol (200-8000 MW) succinate, a tocopherol polyethylene glycol 400 succinate, dl-a-tocopherol polyethyleneglycol 1000 succinate, and d-a-tocopherol polyethyleneglycol 1000 succinate (Vitamin E TPGS, Eastman Chemical Co.) are included. For the practice of this disclosure the mixed racemic forms (e.g. all racemic or dl-) as well as the pure enantiomers (e.g. d-, l- or RRR-) are suitable. Preferred tocol derivative include a-tocopherol esters and a polyethoxylated a-tocopherol esters. More specific preferred tocol derivatives include a-tocopherol, a-tocopherol acetate, a-tocopherol nicotinoate, a-tocopherol succinate, a-tocopherol polyethyleneglycol succinate, a-tocopherol polyethyleneglycol (200-8000 MW) succinate, a-tocopherol polyethylene glycol 400 succinate, a-tocopherol polyethyleneglycol 1000 succinate, dl-a-tocopherol polyethyleneglycol 1000 succinate, or d-a-tocopherol polyethyleneglycol 1000 succinate.

As used herein, the term “long chain lipid solvent” refers to pharmaceutically acceptable lipid solvents comprising twelve or more carbon atoms. In some embodiments, the long chain lipid solvents are able to dissolve the therapeutic amount of API. Examples of long chain lipid solvents include, but are not limited to fatty alcohols, fatty acids, glycerides, vegetable oils, hydro-vegetable oils, animal oils, PEGylated glycerides, Vitamin E derivatives and combinations thereof. In some embodiments, the long chain lipid solvent comprises at least twelve carbon atoms.

In one aspect, pharmaceutical compositions described herein comprise an API (such as clofazimine) or a pharmaceutically acceptable salt thereof and at least one solubilizer. In some embodiments, the pharmaceutical composition comprises an API selected from Table 1 and at least one solubilizer that comprises one or more of the following: (i) petroleum jelly, (ii) one or more alkanes each independently having at least nine carbons, (iii) a fatty alcohol having at least nine carbons; and (iv) a fatty acid having at least nine carbons. In some embodiments, the at least one solubilizer comprises one, two, three, four, five, six or more distinct solubilizers. In some embodiments, the at least one solubilizer comprises one or more of the following: (i) petroleum jelly (i.e., Vaseline), (ii) one or more alkanes each independently having at least nine carbons, (iii) a fatty alcohol having at least nine carbons; (iv) a fatty acid having at least nine carbons, (v) a diol such as propyl glycol, (vi) polyol such as glycerin, (vii) polyethoxylated glycerides such as polyethoxylated monoglycerides, polyethoxylated diglycerides and/or polyethoxylated triglycerides (e.g., polyethoxylated hydrogenated castor oil sold under the tradename Kolliphor® RH40), (viii) polyethylene glycol, (ix) a partially saturated hydrocarbon, or a combination thereof. In some embodiments, the at least one solubilizer comprises a solubilizer described elsewhere herein. In some embodiments, the at least one solubilizer comprises one or more of the following: (i) petroleum jelly, (ii) one or more alkanes each independently having at least nine carbons, (iii) a fatty alcohol having at least nine carbons; and (iv) a fatty acid having at least nine carbons. In some embodiments, the at least one solubilizer comprises one or more of the following: (i) petroleum jelly, (ii) one or more alkanes each independently having at least nine carbons, or both.

In some embodiments, a mass ratio of the API (such as clofazimine) or a pharmaceutically acceptable salt thereof to a herein-described solubilizer is from about 1:10 to about 10,000 in the pharmaceutical composition. In some embodiments, a mass ratio of the API (such as clofazimine) or a pharmaceutically acceptable salt thereof to a herein-described solubilizer is within a range of from about 1:40, 1:50, 1:60, 1:70, 1:80, 1:90, or 1:100 to about 1:200, 1:300, 1:400, 1:500, 1:750, 1:1000, 1:2000, 1:3000, 1:4000 or 1:5000 in the pharmaceutical composition. In some embodiments, a mass ratio of the API (such as clofazimine) or a pharmaceutically acceptable salt thereof to a herein-described solubilizer is about 1:50, 1:70, 1:100, 1:400, 1:1000, 1:2500, or 1:3000.

In some embodiments, pharmaceutical compositions described herein comprise at least one solubilizer, wherein the at least one solubilizer comprises petroleum jelly (i.e., Vaseline). In some embodiments, a mass ratio of the API (such as clofazimine) or a pharmaceutically acceptable salt thereof to petroleum jelly is from about 1:50 to about 1:1000. In some embodiments, a mass ratio of the API (such as clofazimine) or a pharmaceutically acceptable salt thereof to petroleum jelly is from about 1:100 to about 1:250. In some embodiments, the petroleum jelly is present in an amount of from about 20% to about 99% of the total weight of the pharmaceutical composition. In some embodiments, the petroleum jelly is present in an amount of from about 50% to about 95% of the total weight of the pharmaceutical composition. In some embodiments, the petroleum jelly is present in an amount of from about 70% to about 95% of the total weight of the pharmaceutical composition. In some embodiments, the petroleum jelly is present in an amount of from about 70% to about 80% of the total weight of the pharmaceutical composition. In some embodiments, the petroleum jelly is present in an amount of from about 80% to about 90% of the total weight of the pharmaceutical composition. In some embodiments, the petroleum jelly is present in an amount of from about 90% to about 95% of the total weight of the pharmaceutical composition. In some embodiments, the petroleum jelly is present in an amount of at least 90% of the total weight of the pharmaceutical composition. In some embodiments, the petroleum jelly is present in an amount of about 81%, about 82%, about 83%, about 84%, about 85%, about 86%, about 87%, about 88%, about 89%, about 90%, about 90%, about 91%, about 92%, about 93%, about 94%, about 95%, about 96%, about 97%, about 98%, about 99%, or about 99.9% of the total weight of the pharmaceutical composition.

In some embodiments, pharmaceutical compositions described herein comprise at least one solubilizer, wherein the at least one solubilizer comprises one or more alkanes. In some embodiments, each of the one or more alkanes independently having at least nine carbons. In some embodiments, the one or more alkanes comprise nonane, decane, undecane, dodecane, tridecane, tetradecane, pentadecane, hexadecane, heptadecane, octadecane, nonadecane, icosane, heneicosane, docosane, tricosane, tetracosane or a combination thereof, wherein each of the nonane, decane, undecane, dodecane, tridecane, tetradecane, pentadecane, hexadecane, heptadecane, octadecane, nonadecane, icosane, heneicosane, docosane, tricosane, and tetracosane is independently linear or branched. In some embodiments, the one or more alkanes comprise nonane, decane, undecane, dodecane, tridecane, tetradecane, pentadecane, hexadecane, or a combination thereof, wherein each of the nonane, decane, undecane, dodecane, tridecane, tetradecane, pentadecane, and hexadecane is independently linear or branched. In some embodiments, the one or more alkanes are in a liquid state at 25° C. In some embodiments, the one or more alkanes are in a liquid state at 50° C. In some embodiments, the one or more alkanes are in a liquid state at 80° C. In some embodiments, the one or more alkanes are liquid paraffin. In some embodiments, the one or more alkanes (such as liquid paraffin) are present in an amount of from about 0.5% to about 30% of the total weight of the pharmaceutical composition. In some embodiments, the one or more alkanes are present in an amount of from about 2% to about 15% of the total weight of the pharmaceutical composition. In some embodiments, the one or more alkanes are present in an amount of from about 1%, 2%, 3%, 4% or 5% to about 6%, 7%, 8%, 9%, 10%, 11%, 12%, 13%, 14%, 15%, or 20% of the total weight of the pharmaceutical composition. In some embodiments, the one or more alkanes are present in an amount of from about 5% to about 15% of the total weight of the pharmaceutical composition. In some embodiments, the one or more alkanes are present in an amount of from about 5% to about 25% of the total weight of the pharmaceutical composition. In some embodiments, the one or more alkanes are present in an amount of from about 7.5% to about 12.5% of the total weight of the pharmaceutical composition. In some embodiments, the one or more alkanes are present in an amount of from about 2.5% to about 10% of the total weight of the pharmaceutical composition. In some embodiments, the one or more alkanes are present in an amount of from about 5% to about 10% of the total weight of the pharmaceutical composition. In some embodiments, the one or more alkanes are present in an amount of about 5%, about 5.5%, about 6%, about 6.5%, about 7%, about 7.5%, about 8%, about 8.5%, about 9%, about 9.5%, or about 10% of the total weight of the pharmaceutical composition. In some embodiments, the one or more alkanes are present in an amount of about 8%, about 8.1%, about 8.2%, about 8.3%, about 8.4%, about 8.5%, about 8.6%, about 8.7%, about 8.8%, about 8.9%, or about 9% of the total weight of the pharmaceutical composition. In some embodiments, a herein described pharmaceutical composition comprises from about 5 wt % to about 15 wt % of liquid paraffin.

In some embodiments, pharmaceutical compositions described herein comprise at least one solubilizer, wherein the at least one solubilizer comprises a fatty alcohol. In some embodiments, the fatty alcohol is a fatty alcohol of at least nine carbons. In some embodiments, the pharmaceutical composition comprises one or more fatty alcohols. In some embodiments, the fatty alcohol comprises an alcohol having 9, 10, 11, 12, 13, 14, 15, 16, 17, 18, 19, 20, 21, 22, 23, 24, 25, 26, 27, 28, 29, 30, 31, 32, 33, or 34 carbons. In some embodiments, the fatty alcohol comprises an alcohol having 12 to 24 carbons. In some embodiments, the fatty alcohol comprises an alcohol of 9 to 32, 8 to 24, 9 to 18, 12 to 24, or 15 to 24 carbons. In some embodiments, the fatty alcohol is fully saturated or partially saturated. In some embodiments, the fatty alcohol has a linear structure. In some embodiments, the fatty alcohol is branched. In some embodiments, the fatty alcohol is stearyl alcohol. In some embodiments, the fatty alcohol is present in an amount of from about 0.1% to about 15% of the total weight of the pharmaceutical composition. In some embodiments, the fatty alcohol (such as stearyl alcohol) is present in an amount of from about 1% to about 10% of the total weight of the pharmaceutical composition. In some embodiments, the fatty alcohol is present in an amount of from about 1% to about 10%, from about 2% to about 8%, from about 3% to about 6%, or from about 4% to about 5% of the total weight of the pharmaceutical composition. In some embodiments, the fatty alcohol is present in an amount of about 2%, about 2.5%, about 3%, about 3.5%, about 4%, about 4.5%, about 5%, about 5.5%, about 6%, about 6.5%, about 7%, about 7.5%, or about 8% of the total weight of the pharmaceutical composition.

In some embodiments, pharmaceutical compositions described herein comprise at least one solubilizer, wherein the at least one solubilizer comprises a fatty acid. In some embodiments, the fatty acid is a fatty acid of at least nine carbons. In some embodiments, the pharmaceutical composition comprises one or more fatty acids. In some embodiments, the fatty acid comprises 9, 10, 11, 12, 13, 14, 15, 16, 17, 18, 19, 20, 21, 22, 23, 24, 25, 26, 27, 28, 29, 30, 31, 32, 33, or 34 carbons. In some embodiments, the fatty acid comprises an acid of 9 to 32, 8 to 24, 9 to 18, 12 to 24, or 15 to 24 carbons. In some embodiments, pharmaceutical compositions described herein comprise at least one solubilizer, wherein the at least one solubilizer comprises one or more partially saturated hydrocarbon each independently having at least nine carbons. In some embodiments, pharmaceutical compositions described herein comprise at least one solubilizer, wherein the at least one solubilizer comprises a diol such as propyl glycol. In some embodiments, pharmaceutical compositions described herein comprise at least one solubilizer, wherein the at least one solubilizer comprises a polyol such as glycerin. In some embodiments, pharmaceutical compositions described herein comprise at least one solubilizer, wherein the at least one solubilizer comprises one or more polyethoxylated glycerides such as polyethoxylated monoglycerides, polyethoxylated diglycerides and/or polyethoxylated triglycerides (e.g., polyethoxylated hydrogenated castor oil sold under the tradename Kolliphor® RH40). In some embodiments, pharmaceutical compositions described herein comprise at least one solubilizer, wherein the at least one solubilizer comprises polyethylene glycol. In some embodiments, the polyethylene glycol has a weight average molecular weight within a range of from about 500 Da, about 1000 Da, about 1500 Da, about 2000 Da, or about 3000 Da to about 3500 Da, about 4000 Da, about 5000 Da, about 10000 Da, or about 50000 Da. In some embodiments, the polyethylene glycol has a weight average molecular weight of from about 3000 Da to about 4000 Da. In some embodiments, the polyethylene glycol has a weight average molecular weight of from about 2000 Da to about 6000 Da. In some embodiments, the polyethylene glycol has a weight average molecular weight of from about 2000 Da to about 8000 Da. In some embodiments, the polyethylene glycol is PEG 3500. In some embodiments, pharmaceutical compositions described herein comprise at least one solubilizer, wherein the at least one solubilizer comprises.

In some embodiments, a herein described pharmaceutical composition comprises at least one solubilizer, wherein the at least one solubilizer comprises: (i) petroleum jelly in an amount of from about 20% to about 99.9% of the total weight of the pharmaceutical composition, (ii) liquid paraffin in an amount of from about 0% to about 30% of the total weight of the pharmaceutical composition, and (iii) optionally stearyl alcohol in an amount of from about 0.1% to about 15% of the total weight of the pharmaceutical composition. In some embodiments, a herein described pharmaceutical composition consists of an API (such as clofazimine) or a pharmaceutically acceptable salt thereof and at least one solubilizer, wherein the at least one solubilizer comprises: (i) petroleum jelly in an amount of from about 20% to about 99.9% of the total weight of the pharmaceutical composition, and (ii) liquid paraffin in an amount of from about 0% to about 30% of the total weight of the pharmaceutical composition. In some embodiments, the liquid paraffin is in an amount of from about 0.5% to about 30% of the total weight of the pharmaceutical composition.

It will be understood by those of skill in the art that other excipients or components may be added to or mixed with the solubilizing agent(s) to enhance the properties or performance of the solubilizing agent(s) or resulting formulation. Examples of such excipients include, but are not limited to, surfactants, emulsifiers, thickeners, colorants, and the like.

In some embodiments, the API is clofazimine, or a pharmaceutically acceptable salt thereof. In some embodiments, the API is one listed in Table 1, or a pharmaceutically acceptable salt thereof.

### Penetration Enhancers

In some embodiments, the pharmaceutical compositions described herein comprise a lipophilic API, a solubilizer and a penetration enhancer.

The stratum corneum is the outermost layer of the epidermis, consisting of 15-20 layers of dead cells (corneocytes), with no nuclei or organelles, embedded in a lipid matrix of ceramides, cholesterol and fatty acids. Desquamation is the process of cell shedding from the surface of the stratum corneum; cells migrate, over a fourteen day period, through the epidermis towards the surface. The stratum corneum is 10-40 μm thick, shielding the internal structures of the body from external injury, functioning as a barrier to protect underlying tissue from infection, dehydration, chemicals and mechanical stress. Cells of the stratum corneum contain a dense network of keratin that helps keep the skin hydrated by preventing water evaporation. In addition, this layer is responsible for the “spring back” or stretchy properties of skin. An inability to correctly maintain the skin barrier function due to the dysregulation of epidermal components can lead to skin disorders. Skin is attractive target for drug administration (sometimes referred to as topical administration). It offers an easily accessible route without first-pass metabolism, is associated with high patient compliance and through the site of application, drug delivery can be locally directed. However, successful transdermal drug delivery requires overcoming the low permeability of the stratum corneum. One approach to overcome this problem is to employ penetration enhancers.

Penetration enhancers are agents that partition into and interact with the components of the stratum corneum, increasing skin permeability in a temporary, reversible manner. Thus, penetration enhancers reversibly reduce the barrier properties of the stratum corneum to drug penetration, and allow drugs to penetrate more readily into the viable skin tissue and in some cases also into the systemic circulation. Advantages of chemical enhancers over physical enhancers (such as iontophoresis, sonophoresis, electroporation, etc.) are design flexibility, ease of application, the possibility of self-administration and prolonged drug delivery through patches, patient compliance and their incorporation into inexpensive and simple formulations. Table 4 provides a list of various chemical classes of penetration enhancers and exemplary members of the classes for use in a pharmaceutical composition described herein. In some embodiments, a pharmaceutical composition described herein comprises an API of Table 1 and a penetration enhancer of Table 4. In some embodiments, the pharmaceutical composition comprises (a) an API selected from Table 1, (b) at least one solubilizer that comprises one or more of the following: (i) petroleum jelly, (ii) one or more alkanes each independently having at least nine carbons, (iii) a fatty alcohol having at least nine carbons; and (iv) a fatty acid having at least nine carbons, and optionally a penetration enhancer of Table 4.

TABLE 4

| Chemical class: | Enhancer: |
|---|---|
| Alcohols | Short-chain alcohols |
| | Ethanol |
| | Isopropyl alcohol |
| | Long-chain alcohols |
| | Decanol |
| | Octanol |
| | Glycols |
| | Propylene glycol (PG) |
| Amides | Cyclic amides |
| | Laurocapram (i.e., 1-dodecylazacycloheptan-2-one) |
| Fatty acids | Lauric acid |
| | Oleic acid |
| | Linoleic acid |
| Esters | Alkyl esters |
| | Ethyl acetate |
| | Butyl acetate |
| | Methyl acetate |
| | Fatty acid esters |
| | Isopropyl myristate |
| | Isopropyl palmitate |
| Ether alcohols | Transcutol ®(diethylene glycolmonoethyl ether) |
| Surfactants | Anionic surfactants |
| | Sodium lauryl sulphate (SLS) |
| | Cationic surfactants |
| | Benzalkonium chloride |
| | Cetylpyridinium chloride |
| | Cetyltrimethylammonium bromide; |
| | Nonionic surfactants |
| | Polysorbates (Tween ® 20, Tween ® 80, etc.) |
| | Zwitterionic surfactants |
| | Dodecyl betaine |
| Sulphoxides and | Dimethyl sulphoxide (DMSO) |
| analogues | Decylmethyl sulphoxide (DCMS) |
| Essential oils | Eucalyptus |
| | Ylang ylang |
| | Chenopodium |
| Terpenes and | d-Limonene |
| derivatives | 1-Menthol |
| | 1,8-Cineole |
| Pyrrolidones | N-methyl-1-2-pyrrolidone (NMP) |
| | 2-pyrrolidone (2P) |
| Oxazolidines | 4-decyloxazolidin-2-one |
| Phospholipids | Phosphatidylcholine (PC) |
| Enzymes | Acid phosphatase, papain, phospholipase C |

In some embodiments, the penetration enhancers are compounds or mixture of compounds comprising a hydrophobic group (usually a hydrocarbon chain) and a hydrophilic group. They may perform one or more roles including penetration enhancer, solubility enhancer, bioavailability enhancer, stability enhancer, antioxidant and emulsifying agent. Other terms in the art for penetration enhancer include emulsifier, emulsifying agent, surface-active agent, wetting agent, suspending agent and the like. Examples of penetration enhancer include, but are not limited to, phospholipids, sucrose esters of fatty acids, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, sodium dodecyl sulfate, lauromacrogol Arlasolve, Poloxamers, Labrafil, Labrasol, Tween 80 and the like.

In some embodiments, the penetration enhancer is SDS, ethanol, DMSO, polyoxyethylene (80) sorbitan monooleate, polyoxyethylene (20) sorbitan monooleate, octanol, oleic acid or lecithin. In some embodiments, the penetration enhancer is octanol. In some embodiments, the penetration enhancer is lecithin. In some embodiments, the API is clofazimine, or a pharmaceutically acceptable salt thereof. In some embodiments, the API is one listed in Table 1, or a pharmaceutically acceptable salt thereof.

In some embodiments, lipids are fatty acids, and derivatives thereof, that are soluble in non-polar, or organic, solvents while insoluble in water and polar solvents. Categories of lipids include, but are not limited, to fatty acids, phospholipids, sphingolipids, saccharolipids, polyketides, sterol lipids, prenol lipids and the like. In some embodiments, phospholipids are made up of glycerol to which is attached a phosphate group and two fatty acids. Other terms in the art for phospholipids include glycerophospholipids, phosphoglycerides, diacylglycerides and the like. The phosphate group can be unmodified (i.e. in the structure below R=H) or modified by attachment (i.e. in the structure below R≠H) to simple organic molecules such as, but not limited to choline, ethanolamine or serine. Phospholipids may be further modified by substitution onto one or more for the hydrocarbon chains.

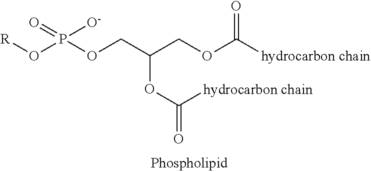

In some embodiments, phospholipids are selected from glycerophospholipid, sphingolipid, and/or phospholipid derivatives. In some embodiments, glycerophospholipids include, but are not limited to phosphatidylcholine, phosphatidyl ethanolamine, phosphatidyl ethanolamine, phosphatidyl serine, phosphatidyl glycerol, diphosphatidylglycerol, phosphatidylinositol, and mixtures thereof. Phospholipid derivatives according to the present disclosure include, but are not limited to dioleoylphosphatidylcholine, dimyristoylphosphatidylcholine, dipentadeanoylphosphatidylcholine, dilauroylphosphatidylchoine, dipalmitoylphosphatidylcholine (DPPC), distearoylphosphatidylcholine (DSPC), diarachidonyiphosphatidylcholine (DAPC), dioleoylphosphatidylethanolamine, dipalmitoylphosphatidylethanolamine (DPPE), and distearoylphosphatidylethanolamine (DSPE), disteraoylphosphatidylglycerol (DSPG), phosphatidylinositol, dipalmitoylphosphatidic acid (DPPA), distearoylphosphatidic acid (DSPA), and the like, and mixtures thereof.

In some embodiments, phosphatidylcholines are phospholipids wherein a choline group (Me₃N⁺—CH₂—CH₂—O—) is attached to the phosphate group.

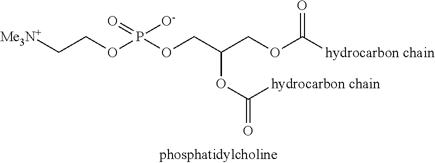

A non-limiting example of a phosphatidylcholine is 1-oleoyl-2-palmitoyl-phosphatidyl choline, as shown below:

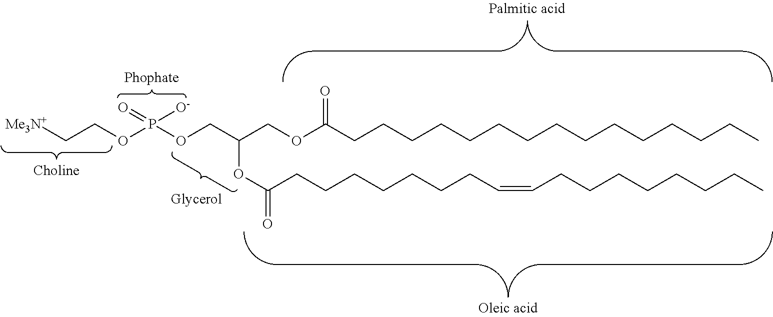

In some embodiments, lecithin is a mixture of phospholipids. Lecithins can be isolated from various sources including, but not limited to eggs, soybeans, milk, marine sources, rapeseed, cottonseed and sunflower.

In some embodiments, the penetration enhancer is a phospholipid. In some embodiments, the phospholipid is phosphatidylcholine. In some embodiments, the phospholipid is a mixture comprising phosphatidylcholine. In some embodiments, the penetration enhancer is lecithin. In some embodiments, the phosphatidylcholine is lecithin. In some embodiments, the lecithin contains more than 25% of phosphatidylcholine. In some embodiments, the lecithin contains more than 80% of phosphatidylcholine. In some embodiments, the phosphatidylcholine is from egg origin. In some embodiments, the phosphatidylcholine is from or soybean origin. In some embodiments, the API is clofazimine, or a pharmaceutically acceptable salt thereof. In some embodiments, the API is one listed in Table 1, or a pharmaceutically acceptable salt thereof.

In one aspect, pharmaceutical compositions described herein comprise an API (such as clofazimine) or a pharmaceutically acceptable salt thereof, at least one solubilizer, and a stratum corneum penetration enhancer. In some embodiments, the stratum corneum penetration enhancer is polyethoxylated sorbitan monooleate, Laurocapram, phospholipids, ethanol, pegylated fatty acid glyceride, or a combination thereof. In some embodiments, the stratum corneum penetration enhancer is selected from Table 4. In some embodiments, the stratum corneum penetration enhancer is lecithin. In some embodiments, the stratum corneum penetration enhancer is present in an amount of from about 1% to about 20% of the total weight of the pharmaceutical composition.

In some embodiments, the penetration enhancer comprises about 1% to about 20% of the total weight of a herein described pharmaceutical composition. In some embodiments, the penetration enhancer comprises about 1% to about 5%, about 5% to about 10%, about 10% to about 15%, about 15% to about 20%, about 2% to about 12%, or about 7.5% to about 12.5% of the total weight of a herein described pharmaceutical composition. In some embodiments, the penetration enhancer comprises about 1% of the total weight of a herein described pharmaceutical composition. In some embodiments, the penetration enhancer comprises about 1% of the total weight of the composition. In some embodiments, the penetration enhancer comprises about 2% of the total weight of the composition. In some embodiments, the penetration enhancer comprises about 3% of the total weight of the composition. In some embodiments, the penetration enhancer comprises about 4% of the total weight of the composition. In some embodiments, the penetration enhancer comprises about 5% of the total weight of the composition. In some embodiments, the penetration enhancer comprises about 6% of the total weight of the composition. In some embodiments, the penetration enhancer comprises about 7% of the total weight of the composition. In some embodiments, the penetration enhancer comprises about 8% of the total weight of the composition. In some embodiments, the penetration enhancer comprises about 9% of the total weight of the composition. In some embodiments, the penetration enhancer comprises about 10% of the total weight of the composition. In some embodiments, the penetration enhancer comprises about 11% of the total weight of the composition. In some embodiments, the penetration enhancer comprises about 12% of the total weight of the composition. In some embodiments, the penetration enhancer comprises about 13% of the total weight of the composition. In some embodiments, the penetration enhancer comprises about 14% of the total weight of the composition. In some embodiments, the penetration enhancer comprises about 15% of the total weight of the composition. In some embodiments, the penetration enhancer comprises about 16% of the total weight of the composition. In some embodiments, the penetration enhancer comprises about 17% of the total weight of the composition. In some embodiments, the penetration enhancer comprises about 18% of the total weight of the composition. In some embodiments, the penetration enhancer comprises about 19% of the total weight of the composition. In some embodiments, the penetration enhancer comprises about 20% of the total weight of the composition. In some embodiments, the penetration enhancer comprises about 25% of the total weight of the composition. In some embodiments, the penetration enhancer comprises about 30% of the total weight of the composition. In some embodiments, the API is clofazimine, or a pharmaceutically acceptable salt thereof. In some embodiments, the API is one listed in Table 1, or a pharmaceutically acceptable salt thereof.

In some embodiments, the pharmaceutical compositions comprise a lipophilic API, a solubilizer, and a penetration enhancer. In some embodiments, the ratio by weight of the API to (solubilizer+penetration enhancer) is from about 1:25 to 1:2500. In some embodiments, the ratio by weight of the API to (solubilizer+penetration enhancer) is from about 1:50 to 1:2000. In some embodiments, the ratio by weight of the API to (solubilizer+penetration enhancer) is from about 1:100 to 1:1000. In some embodiments, the ratio by weight of the API to (solubilizer+penetration enhancer) is from about 1:100 to 1:500. In some embodiments, the ratio by weight of the API to (solubilizer+penetration enhancer) is about 1:250. In some embodiments, the ratio by weight of the penetration enhancer to the solubilizer is from about 1:1 to 1:50. In some embodiments, the ratio by weight of the penetration enhancer to the solubilizer is from about 1:2 to 1:20. In some embodiments, the ratio by weight of the penetration enhancer to the solubilizer is from about 1:2 to 1:10. In some embodiments, the ratio by weight of the penetration enhancer to the solubilizer is from about 1:3 to 1:5. In some embodiments, the ratio by weight of the penetration enhancer to the solubilizer is about 1:4. In some embodiments, the API is clofazimine, or a pharmaceutically acceptable salt thereof. In some embodiments, the API is one listed in Table 1, or a pharmaceutically acceptable salt thereof.

### Gelling Agents

In some embodiments, pharmaceutical compositions described herein comprise a lipophilic API (such as clofazimine), at least one solubilizer and optionally a penetration enhancer. In some embodiments, the pharmaceutical compositions may optionally comprise one or more gelling agents.

Gels are transparent or translucent, semi-solid, highly cross-linked polymeric matrices. They comprise small amounts of solid, gelling agent, dispersed in a relatively large amount of liquid; although most of their content is liquid, they demonstrate more solid like properties. When dispersed in an appropriate solvent, gelling agents merge or entangle to form a three-dimensional network structure which limits fluid flow by entrapment and immobilization of the solvent molecules. A gel is formed when the particles of a gelling agent cross-link, via chemical bonds or physical interactions (e.g. hydrogen bonds), into a three dimensional network that behaves like a solid, even though it is mostly liquid.

Creams, ointments, gels, pastes and the like have been used as topical dosage forms. Topical gel formulations may provide advantages as drug delivery systems, such as: they are less greasy than creams or ointments; they possess better application properties; excess gel is easily removed from the skin; they are typically more stable then creams and ointments.

Various types of gels exist, including, but not limited to:
Hydrogels—consisting of high water content and are superabsorbent.
Organogels—noncrystalline, nonglassy, thermoplastic solid materials composed of a liquid organic phase entrapped in a three-dimensionally, cross-linked network.
Xerogels—solids formed from gels that do not shrink in the process, generally retaining a high degree of porosity. Examples include rubber and gelatin
Aerogels—low-density materials created when the liquid component of a gel is replaced by a gas (e.g. nitrogen, air).
   Others include inorganic, organic, biopolymer, monomer gels, emulgels, in situ gels, micro-emulsion gels, and the like.

Polyacrylic acid (PAA or carbomer or carbopol) is a synthetic, high molecular weight polymer of acrylic acid. The chemical name is poly(1-carboxyethylene).

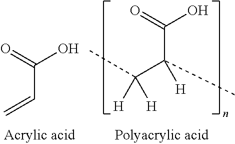

Carbomers are water soluble, and can absorb and retain water, able to swell up to 1000 times their original volume, and as such may be used as gelling agents. Carbomers are often associated with codes (such as, for example, 940, 934, 940, 941, 934P and the like) reflecting the molecular weight and the composition of the polymer.

In a water solution at neutral pH, carbomers are anionic polymers, i.e. many of the acid side chains lose their protons and acquire a negative charge. Carbomers may be used as alkali metal or ammonium salts, e.g. sodium polyacrylate. In the dry powder form, the positively charged sodium ions are bound to the polyacrylate. In aqueous solution, the sodium ions move freely, since they are replaced by positively charged hydrogen ions. Instead of an organized polymeric chain, this leads to a swollen gel capable of absorbing high volumes of water.

Gelling may be effected via a two-step process. First, the carbomer is hydrated and dispersed throughout the aqueous media. Then the solution is neutralized by addition of a base. Neutralizing agents include sodium hydroxide, potassium hydroxide, ammonium hydroxide, or organic amine such as triethanolamine (TEA). Neutralizing 1 g of carbomer to pH 7 requires about 0.01 equivalent of base.

In some embodiments, the pharmaceutical compositions described herein are gels. In some embodiments, the pharmaceutical compositions described herein comprise a lipophilic API, a solubilizer, a penetration enhancer and a gelling agent.

In some embodiments, the gelling agent is a carbomer, polyacrylic acid, hyaluronic acid, polyvinyl alcohol, methylcellulose, ethylcellulose, hydroxyethyl cellulose, hydroxypropyl cellulose, hydroxyporplymethyl cellulose (HPMC), carboxymethyl cellulose (CMC), sodium CMC, hypromellose, carbopol, polyvinyl alcohol, polyvinylmethacrylic acid, polaxamer, adenosylcobalamin, gellan gum, pectin, high methoxyl pectin (HMP), low methoxyl pectin (LMP), tragacanth, acacia, or combinations thereof.

In some embodiments, the gelling agent is a carbomer, polyacrylic acid, polyvinyl alcohol, cellulose, methyl cellulose, ethyl cellulose, hypromellose, hydroxyethyl cellulose, hyaluronic acid or a combination thereof. In some embodiments, the gelling agent is a carbomer. In some embodiments, the gelling agent is carbomer 940. In some embodiments, the API is clofazimine, or a pharmaceutically acceptable salt thereof. In some embodiments, the API is one listed in Table 1, or a pharmaceutically acceptable salt thereof.

In some embodiments, the gelling agent comprises about 0.1% to 5% of the total weight of the composition. In some embodiments, the gelling agent comprises about 0.1% to about 0.5%, about 0.1% to about 1%, about 0.2% to about 1%, about 0.5% to about 2.5%, about 0.5% to about 5%, about 1% to about 2%, about 1% to about 5%, or about 0.5% to about 3% of the total weight of the composition. In some embodiments, the gelling agent comprises about 0.1% of the total weight of the composition. In some embodiments, the gelling agent comprises about 0.2% of the total weight of the composition. In some embodiments, the gelling agent comprises about 0.3% of the total weight of the composition. In some embodiments, the gelling agent comprises about 0.4% of the total weight of the composition. In some embodiments, the gelling agent comprises about 0.5% of the total weight of the composition. In some embodiments, the gelling agent comprises about 0.6% of the total weight of the composition. In some embodiments, the gelling agent comprises about 0.7% of the total weight of the composition. In some embodiments, the gelling agent comprises about 0.75% of the total weight of the composition. In some embodiments, the gelling agent comprises about 0.8% of the total weight of the composition. In some embodiments, the gelling agent comprises about 0.9% of the total weight of the composition. In some embodiments, the gelling agent comprises about 1.0% of the total weight of the composition. In some embodiments, the gelling agent comprises about 1.2% of the total weight of the composition. In some embodiments, the gelling agent comprises about 1.3% of the total weight of the composition. In some embodiments, the gelling agent comprises about 1.4% of the total weight of the composition. In some embodiments, the gelling agent comprises about 1.5% of the total weight of the composition. In some embodiments, the gelling agent comprises about 1.6% of the total weight of the composition. In some embodiments, the gelling agent comprises about 1.7% of the total weight of the composition. In some embodiments, the gelling agent comprises about 1.8% of the total weight of the composition. In some embodiments, the gelling agent comprises about 1.9% of the total weight of the composition. In some embodiments, the gelling agent comprises about 2.0% of the total weight of the composition. In some embodiments, the gelling agent comprises less than 2.0% of the total weight of the composition. In some embodiments, the gelling agent comprises less than 1.5% of the total weight of the composition. In some embodiments, the gelling agent comprises less than 1% of the total weight of the composition. In some embodiments, the gelling agent comprises less than 0.75% of the total weight of the composition. In some embodiments, the API is clofazimine, or a pharmaceutically acceptable salt thereof. In some embodiments, the API is one listed in Table 1, or a pharmaceutically acceptable salt thereof.

In some embodiments, the ratio by weight of the gelling agent to the (API+solubilizer+penetration enhancer) is about 1:5. In some embodiments, the ratio by weight of the gelling agent to the (API+solubilizer+penetration enhancer) is about 1: 10. In some embodiments, the ratio by weight of the gelling agent to the (API+solubilizer+penetration enhancer) is about 1:50. In some embodiments, the ratio by weight of the gelling agent to the (API+solubilizer+penetration enhancer) is about 1:250. In some embodiments, the ratio by weight of the gelling agent to the (API+solubilizer+penetration enhancer) is about 1:500. In some embodiments, the API is clofazimine, or a pharmaceutically acceptable salt thereof. In some embodiments, the API is one listed in Table 1, or a pharmaceutically acceptable salt thereof.

### Neutralizing Agents

In some embodiments, pharmaceutical compositions described herein comprise a lipophilic API, at least one solubilizer and optionally a penetration enhancer. In some embodiments, the pH of the pharmaceutical composition is no more than 9.5. In some embodiments, the pH is no more than 9.0. In some embodiments, the pH is no more than 8.5. In some embodiments, the pH is no more than 8.0. In some embodiments, the pH is no more than 7.5. In some embodiments, the pH is about 6-8. In some embodiments, the pH is about 6-7. In some embodiments, the pH is about 5-7. In some embodiments, the pH is about 5-6. In some embodiments, the pH is about 4-9. In some embodiments, the pH is about 6.5 to 7.5. In some embodiments, the pH is below 4 or 5. In some embodiments, the pH is above 6, 7, 8, or 9. In some embodiments, the pH is about 7. In some embodiments, the pH of the pharmaceutical composition is adjusted to the desired pH by addition of a neutralizing agent (or pH adjuster). In some embodiments, the neutralizing agent is triethanolamine, ethylenediamine, sodium citrate, sodium hydroxide, potassium hydroxide, ammonium hydroxide, sodium hydrogen phosphate or a combination thereof. In some embodiments, the neutralizing agent is triethanolamine. In some embodiments, the API is clofazimine, or a pharmaceutically acceptable salt thereof. In some embodiments, the API is one listed in Table 1, or a pharmaceutically acceptable salt thereof. In some embodiments, the pharmaceutical compositions described herein comprise a lipophilic API, a solubilizer, a penetration enhancer, a gelling agent and a neutralizing agent.

### Water

In some embodiments, the pharmaceutical compositions described herein comprise a lipophilic API, a solubilizer, optionally a penetration enhancer, and optionally water. In some embodiments, the pharmaceutical compositions described herein comprise a lipophilic API, a solubilizer, optionally a penetration enhancer, optionally a gelling agent, optionally a neutralizing agent and optionally water. In some embodiments, the pharmaceutical compositions comprise from about 30% to about 95% water. In some embodiments, the pharmaceutical compositions comprise from about 40% to about 90% water by weight. In some embodiments, the pharmaceutical compositions comprise from about 50% to about 90% water by weight. In some embodiments, the pharmaceutical compositions comprise from about 60% to about 80% water by weight. In some embodiments, the pharmaceutical compositions comprise from about 70% to about 80% water by weight. In some embodiments, the pharmaceutical compositions comprise from about 75% to about 80% water by weight. In some embodiments, the pharmaceutical compositions comprise about 75% water by weight. In some embodiments, the pharmaceutical compositions comprise less than 10%, less than 5%, less than 2%, less than 1%, less than 0.5% or less than 0.1% of water by weight. In some embodiments, the pharmaceutical composition is free of water. In some embodiments, the API is clofazimine, or a pharmaceutically acceptable salt thereof. In some embodiments, the API is one listed in Table 1, or a pharmaceutically acceptable salt thereof. In some embodiments, pharmaceutical compositions described herein are free of water.

### Other Additives

In some embodiments, pharmaceutical compositions described herein comprise a lipophilic API, at least one solubilizer, optionally a penetration enhancer, and optionally one or more additives. In some embodiments, the pharmaceutical compositions described herein comprise a lipophilic API, a solubilizer, a penetration enhancer, a gelling agent, a neutralizing agent, optionally water and, optionally, one or more additives. In some embodiments, the pharmaceutical compositions further comprise a moisturizing agent. In some embodiments, the moisturizing agent is glycerin, urea, allantoin or a combination thereof. In some embodiments, the API is clofazimine, or a pharmaceutically acceptable salt thereof. In some embodiments, the API is one listed in Table 1, or a pharmaceutically acceptable salt thereof.

In some embodiments, the pharmaceutical compositions described herein further comprise a preservative. In some embodiments, the preservative is sodium benzoate, chlorobutanol, paraben or sorbic acid. In some embodiments, the API is clofazimine, or a pharmaceutically acceptable salt thereof. In some embodiments, the API is one listed in Table 1, or a pharmaceutically acceptable salt thereof.

Compositions described herein may optionally comprise an antioxidant. In some embodiments, antioxidants are used to reduce the oxidation or degradation of API and excipients in the composition. In some embodiments, the antioxidant is selected from ascorbic acid, ascorbyl palmitate, butylated hydroxy anisole (BHA), butylated hydroxyl toluene (BHT), citric acid, cysteine, gallic acid, guaiac resin, methionine, phosphoric acid, potassium metabisulfite, propyl gallate, sesamol, sodium edate, sodium sulfite, sodium bisulfite, sodium metabisulfite, sodium thiosulfate, terbuteryl hydroquinone, tartaric acid, tertiary butyl hydroquinone, Vitamin E (tocopherol), thioglycerol, thioglycollic acid, or a combination thereof. In some embodiments, the antioxidant is vitamin E, butylated hydroxytoluene, butylated hydroxyanisole, or a combination thereof. In some embodiments, the antioxidant is vitamin E, 2,6-di-tert-butyl-4-methylphenol, butylhydroxyanisole, ascorbyl palmitate, tert-butyl hydroquinone or a combination thereof.

In some embodiments, the one or more additives are present in an amount of less than 2.0% of the total weight of a herein described pharmaceutical composition. In some embodiments, the one or more additives are present in an amount less than 1.75% of the total weight of the composition. In some embodiments, the one or more additives are present in an amount less than 1.5% of the total weight of the composition. In some embodiments, the one or more additives are present in an amount less than 1.25% of the total weight of the composition. In some embodiments, the one or more additives are present in an amount less than 1.0% of the total weight of the composition. In some embodiments, the one or more additives comprise about 0.1% of the total weight of the composition. In some embodiments, the one or more additives comprise about 0.2% of the total weight of the composition. In some embodiments, pharmaceutical compositions described herein include a lipophilic API, a long chain fatty acid or a long chain fatty acid glyceride, a phospholipid and, optionally, and one or more additives. In some embodiments, the one or more additives comprise about 0.3% of the total weight of the composition. In some embodiments, the one or more additives comprise about 0.4% of the total weight of the composition. In some embodiments, the one or more additives comprise about 0.5% of the total weight of the composition. In some embodiments, the one or more additives comprise about 0.6% of the total weight of the composition. In some embodiments, the one or more additives comprise about 0.7% of the total weight of the composition. In some embodiments, the one or more additives comprise about 0.8% of the total weight of the composition. In some embodiments, the one or more additives comprise about 0.9% of the total weight of the composition. In some embodiments, the one or more additives comprise about 1.0% of the total weight of the composition. In some embodiments, the one or more additives comprise about 1.1% of the total weight of the composition. In some embodiments, the one or more additives comprise about 1.2% of the total weight of the composition. In some embodiments, the one or more additives comprise about 1.25% of the total weight of the composition. In some embodiments, the one or more additives are present in an amount of from about 0.01% to about 2% of the total weight of the composition. In some embodiments, the one or more additives are present in an amount of from about 0.1% to about 1.75% of the total weight of the composition. In some embodiments, the one or more additives are present in an amount of from about 0.2% to about 1.5% of the total weight of the composition. In some embodiments, the one or more additives are present in an amount of from about 0.3% to about 1.25% of the total weight of the composition. In some embodiments, the one or more additives are present in an amount of from about 0.4% to about 1.1% of the total weight of the composition.

In some embodiments, other additives conventionally used in pharmaceutical compositions may be included, and these additives are well known in the art. Such additives include, but are not limited to, anti-adherents (anti-sticking agents, glidants, flow promoters, lubricants) (e.g., talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate) anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4hydroxymethyl-2,6-di-tert-butyl phenol, tocopherol, etc.), binders (adhesives), i.e., agents that impart cohesive properties to powdered materials through particle-particle bonding (e.g., matrix binders (dry starch, dry sugars), film binders (PVP, starch paste, celluloses, bentonite, sucrose)), chemical binders (e.g., polymeric cellulose derivatives, such as carboxy methyl cellulose, HPC, HPMC, etc., sugar syrups, corn syrup, water soluble polysaccharides (e.g., acacia, tragacanth, guar, alginates, etc), gelatin, gelatin hydrolysate, agar, sucrose, dextrose, non-cellulosic binders (e.g., PVP, PEG, vinyl pyrrolidone copolymers, pregelatinized starch, sorbitol, glucose, etc.), bufferants, where the acid is a pharmaceutically acceptable acid (e.g., hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, etc) and where the base is a pharmaceutically acceptable base (e.g., an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a pharmaceutically acceptable salt of acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, parabromophenylsulfonic acid, propionic acid, p-toluenesulfonic acid, salicylic acid, stearic acid, succinic acid, tannic acid, tartaric acid, thioglycolic acid, toluenesulfonic acid, and uric acid, chelating agents (e.g., EDTA and EDTA salts), coagulants (e.g., alginates) colorants or opaquants, (e.g., titanium dioxide, food dyes, lakes, natural vegetable colorants, iron oxides, silicates, sulfates, magnesium hydroxide and aluminum hydroxide), coolants (e.g. halogenated hydrocarbons (e.g., trichloroethane, trichloroethylene, dichloromethane, fluorotrichloromethane), diethylether and liquid nitrogen) cryoprotectants (e.g., trehelose, phosphates, citric acid, tartaric acid, gelatin, dextran, mannitol, etc.), diluents or fillers (e.g., lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose disintegrants or super disintegrants (e.g., croscarmellose sodium, starch, starch derivatives, clays, gums, cellulose, cellulose derivatives, alginates, crosslinked polyvinylpyrrolidone, sodium starch glycolate and microcrystalline cellulose), hydrogen bonding agents, (e.g., magnesium oxide), flavorants or desensitizers (e.g., spray-dried flavors, essential oils and ethyl vanillin), ion-exchange resins (e.g., styrene/divinyl benzene copolymers, and quaternary ammonium compounds), plasticizers (e.g., polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate), preservatives (e.g., ascorbic acid, boric acid, sorbic acid, benzoic acid, and salts thereof, parabens, phenols, benzyl alcohol, and quaternary ammonium compounds), solvents (e.g., alcohols, ketones, esters, chlorinated hydrocarbons and water) sweeteners, including natural sweeteners (e.g., maltose, sucrose, glucose, sorbitol, glycerin and dextrins), and artificial sweeteners (e.g., aspartame, saccharine and saccharine salts) and thickeners (viscosity modifiers, thickening agents) (e.g., sugars, polyvinylpyrrolidone, cellulosics, polymers and alginates).

Additives can also be materials such as proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, carnuaba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics (e.g., dicalcium phosphate, hydroxyapitite, tricalcium phosphate, talc and titania), polyols (e.g., mannitol, xylitol and sorbitol polyethylene glycol esters) and polymers (e.g., alginates, poly(lactide coglycolide), gelatin, crosslinked gelatin and agar-agar).

It should be appreciated that there is considerable overlap between the above listed components in common usage, since a given component is often classified differently by different practitioners in the field, or is commonly used for any of several different functions, or may have differing functions depending on the levels in the composition. Thus, the above-listed components should be taken as merely exemplary, and not limiting, of the types of components that can be included in compositions of the present disclosure.

Other agents suitable for inclusion in the pharmaceutical composition include, humectants, such as propylene glycol, glycerin, butylene glycol, sorbitol, triacetin, and mixtures thereof; fragrances, such as lavender oil, rose oil, lemon oil, almond oil, other FDA-approved fragrances, and mixtures thereof; cooling agents, such as menthol, e.g., 1-menthol and dl-menthol; camphor, e.g., d-camphor and dl-camphor; borneol, e.g., d-borneol and dl-borneol; and mixtures thereof.

### Stabilizers

In some embodiments, the compositions disclosed herein comprise a stabilizer. In some embodiments, any of the solubilizers, penetration enhancers, gelling agents, or additives serve the additional function of a stabilizer. In some embodiments, a stabilizer decreases the decomposition of the active pharmaceutical ingredient in the composition, such as the APIs in Table 1.

In some embodiments, the compositions disclosed herein comprise clofazimine. Clofazimine degrades by oxidation and hydrolysis and requires stabilization for storage. In some embodiments, the stabilizer is selected from labrasol, transcutol, propyl glycol, oleic acid, glycerin, PEG 3350, Polyoxyl 40 Hydrogenated castor oil (RH 40), stearyl alcohol, vaseline, and liquid paraffin. In some embodiments, the stabilizer is selected from propyl glycol, glycerin, PEG 3350, Polyoxyl 40 Hydrogenated castor oil (RH 40), stearyl alcohol, vaseline, and liquid paraffin. In some embodiments, the stabilizer is selected from stearyl alcohol, vaseline, and liquid paraffin.

### Stability

Topical formulations described herein are chemically and physically stable at various storage conditions for a prolonged period of time. The term “stable,” as used herein, can refer to a formulation that maintains its appearance (e.g., coloring, uniformity, no agglomeration) after a given storage period. The term “stable,” as used herein, can also refer to a formulation that maintains the API content, e.g., from about 90% to about 110% of the initial API amount or from about 95% to about 105% of the initial API amount after a given storage period. The term “stable,” as used herein, can also refer to a formulation that retains no more than 2% w/w of total impurity or less than 1% w/w of single impurity based on the weight of the initial API amount after a given storage period. The term “stable,” as used herein, can also refer to a formulation that retains a microbial level below a prescribed limit after a given storage period. In some embodiments, that period of time or storage period is for at least 7 days, at least 14 days, at least 15 days, at least 1 month, at least 2 months, at least 3 months, at least 6 months, at least 9 months, at least 12 months, at least 15 months, at least 18 months, or at least 24 months. In some embodiments, a formulation described herein is stable for at least 6 months. In some embodiments, a formulation described herein is stable for at least 12 months. In some embodiments, a formulation described herein is stable for at least 18 months. In some embodiments, a formulation described herein is stable for at least 24 months. In some embodiments, the storage condition is at refrigerated temperature such as about 4° C. In some embodiments, the storage condition is at about 40° C. In some embodiments, the storage condition is at about 25° C. In some embodiments, the storage condition is an accelerated condition of about 40° C. and about 75% RH. In some embodiments, the storage condition is about 25° C. and about 60% RH.

In some embodiments, a formulation described herein has assay values of no less than 90% and no more than 110% of labeled amount of API (e.g., clofazimine or an API of table 1). In some embodiments, the assay value can be measured using an HPLC method. In some embodiments, a formulation described herein has assay values of about 85% to about 115%, about 90% to about 110%, about 95% to about 105%, about 98% to about 102%, or 99% to about 101% after a given storage period of labeled API (e.g., clofazimine). In some embodiments, a formulation described herein has assay values of about 90% to about 110% after a given storage period of labeled API (e.g., clofazimine) when stored at 40° C. In some embodiments, a formulation described herein has assay values of about 90% to about 110% after a given storage period of labeled API when stored at 25° C. In some embodiments, the API in a stable formulation described herein has at least about 85% w/w, at least about 86% w/w, at least about 87% w/w, at least about 88% w/w, at least about 89% w/w, at least about 90% w/w, at least about 91% w/w, at least about 92% w/w, at least about 93% w/w, at least about 94% w/w, at least about 95% w/w, at least about 96% w/w, at least about 97% w/w, at least about 98% w/w, at least about 99% w/w, or at least about 100% w/w of the initial API amount after a given storage period. In some embodiments, a formulation described herein retains about 90% to about 110% w/w of the initial API amount after a given storage period. In some embodiments, a formulation described herein retains about 95% to about 105% w/w of the initial API amount after a given storage period. In some embodiments, a formulation described herein retains at least about 85% w/w, at least about 86% w/w, at least about 87% w/w, at least about 88% w/w, at least about 89% w/w, at least about 90% w/w, at least about 91% w/w, at least about 92% w/w, at least about 93% w/w, at least about 94% w/w, at least about 95% w/w, at least about 96% w/w, at least about 97% w/w, at least about 98% w/w, at least about 99% w/w, or at least about 100% w/w of the initial API amount after a given storage period. In some embodiments, the API in a stable formulation described herein has at most about 115% w/w, at most about 110% w/w, at most about 109% w/w, at most about 108% w/w, at most about 107% w/w, at most about 106% w/w, at most about 105% w/w, at most about 104% w/w, at most about 103% w/w, at most about 102% w/w, at most about 101% w/w, or at most about 100% w/w of the initial API amount after a given storage period. In some embodiments, the API in a stable formulation described herein is within the range of about 100±15% w/w, about 100±10% w/w, about 100±9% w/w, about 100±8% w/w, about 100±7% w/w, about 100±6% w/w, about 100±5% w/w, about 100±4% w/w, about 100±3% w/w, about 100±2.5% w/w, about 100±2.0% w/w, about 100±1.5% w/w, about 100±1.0% w/w, or about 100±0.5% w/w of the initial API amount after a given storage period. In some embodiments, a formulation described herein has an assay value of about 90% to about 110% of the initial API amount after a given storage period when stored at 25° C. In some embodiments, a formulation described herein has an assay value of about 90% to about 110% of the initial API amount after a given storage period when stored at 40° C. In some embodiments, a formulation described herein has an assay value of about 95% to about 105% of the initial API amount after a given storage period when stored at 25° C. and/or at 40° C. In some embodiments, a formulation described herein has an assay value of about 98% to about 102% of the initial API amount after a given storage period when stored at 25° C. and/or at 40° C. In some embodiments, the API amount is determined by liquid chromatography such as high performance liquid chromatography (HPLC). In some embodiments, the total impurities in a stable formulation described herein is no more than about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4 w/w, or about 2.5% w/w based on the weight of the initial amount of the API after a given storage period. In some embodiments, the total impurities in a stable formulation described herein is no more than about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, or about 10% w/w based on the weight of the formulation after a given storage period. In some embodiments, the storage period is at least 6 months, at least 9 months, at least 12 months or any period in-between. In some embodiments, the storage period is at least 6 months. In some embodiments, the storage period is at least 9 months. In some embodiments, the storage condition has a relative humidity of about 60% In some embodiments, the storage condition has a relative humidity of about 75%. In some embodiments, the storage condition has a temperature of about 40° C. In some embodiments, the storage condition has a temperature of about 25° C. In some embodiments, the API is clofazimine. In some embodiments, the API is an API of Table 1.

In some embodiments, the compositions disclosed herein comprise clofazimine and at least one stabilizer. In some embodiments, the composition is stable for 3 days at 60° C. In some embodiments, the composition is stable for 1, 2, 3, 4, 5, 6, 7, 8, 9, or more months at room temperature and various humidity levels (such as 60% and 75%). In some embodiments, the composition is stable for 1, 2, 3, 4, 5, 6, 7, 8, 9, or more months at elevated temperatures and elevated humidities (such as 75%). In some embodiments, the amount of total impurities resulting from degradation is no more than about 0.01%, 0.05%, 0.1%, 0.2%, 0.3%, 0.4%, 0.5%. 0.6%, 0.7%, 0.8%, 0.9%, 1.0%, 2.0%, 3.0% or 5.0% based on the weight of the initial API amount or the weight of formulation after a given storage period. In some embodiments, the amount of impurities resulting from degradation is about 0.01% to about 5%. In some embodiments, the amount of impurities resulting from degradation is about 0.01% to about 0.05%, about 0.01% to about 0.1%, about 0.01% to about 0.25%, about 0.01% to about 0.5%, about 0.01% to about 0.75%, about 0.01% to about 1%, about 0.01% to about 2%, about 0.01% to about 3%, about 0.01% to about 5%, about 0.1% to about 0.25%, about 0.1% to about 0.5%, about 0.1% to about 0.75%, about 0.1% to about 1%, about 0.1% to about 2%, about 0.1% to about 3%, or about 0.1% to about 5% based on the weight of the initial API amount or the weight of the formulation after a given storage period. In some embodiments, the amount of total impurities resulting from degradation is less than about 0.2% based on the weight of the initial API amount over 9 months. In some embodiments, the amount of total impurities resulting from degradation is less than about 0.15% over 9 months. In some embodiments, the amount of total impurities resulting from degradation is no more than about 2% based on the weight of the initial API amount after a given storage period. In some embodiments, the amount of total impurities resulting from degradation is no more than about 1% based on the weight of the initial API amount after a given storage period. In some embodiments, the amount of a single impurity resulting from degradation is no more than about 1% based on the weight of the initial API amount after a given storage period. In some embodiments, the amount of a single impurity resulting from degradation is no more than about 0.5% based on the weight of the initial API amount after a given storage period. In some embodiments, the total impurity is determined by liquid chromatography, such as high performance liquid chromatography (HPLC). In some embodiments, the storage period is at least 6 months, at least 9 months, at least 12 months or any period in-between. In some embodiments, the storage period is at least 6 months. In some embodiments, the storage period is at least 9 months. In some embodiments, the storage condition has a relative humidity of about 60% In some embodiments, the storage condition has a relative humidity of about 75%. In some embodiments, the storage condition has a temperature of about 40° C. In some embodiments, the storage condition has a temperature of about 25° C. In some embodiments, the API is clofazimine. In some embodiments, the API is an API of Table 1.

In some embodiments, a formulation described herein is physically stable over a storage period. In some embodiments, the API of the formulation remains dissolved with no crystalline formation as observed visually over a storage period of at least 1 month, 6 months, 9 months, 12 months, or 12 months. In some embodiments, the API of the formulation remains dissolved with no crystalline formation as observed by polarizing microscope over a storage period of at least 1 month, 6 months, 9 months, 12 months, or 12 months. In some embodiments, the storage period is at least 6 months. In some embodiments, the storage period is at least 9 months. In some embodiments, the storage condition has a temperature of about 40° C. In some embodiments, the storage condition has a temperature of about 25° C. In some embodiments, the API is clofazimine. In some embodiments, the API is an API of Table 1.

### Methods of Administration

In some embodiments, pharmaceutical compositions described herein are for topical administration. In some embodiments, the pharmaceutical compositions described herein are for transdermal administration. In some embodiments, the API is clofazimine, or a pharmaceutically acceptable salt thereof. In some embodiments, the API is one listed in Table 1, or a pharmaceutically acceptable salt thereof. Accordingly, described herein are methods of treating a disease or condition to a subject in need thereof, wherein the method comprises applying a herein described topical formulation to the subject. Further provided herein are methods of delivering a herein described API (such as clofazimine or a pharmaceutically acceptable salt thereof) to the skin of a subject for example, to an epidermis layer and/or a dermis layer of the skin, by administering a herein described topical formulation.

Topical or transdermal administration refers to delivery of an API by passage into and through the skin or mucosal tissue. Hence, the terms “transdermal” and “transmucosal” are used interchangeably unless specifically stated otherwise. Likewise, the terms “skin,” “derma,” “epidermis,” “mucosa” and the like will also be used interchangeably unless specifically stated otherwise. A topical pharmaceutical composition is applied to a particular place on or in the body. Most often topical administration refers to application to body surfaces such as the skin or mucous membranes. Topical medications may be epicutaneous, meaning they are applied directly to the skin. In some instances, a formulation may be applied directly to the skin in a free form, which is sufficient to effect transdermal delivery of the API without the use of structures, such as a backing member, etc. In some instances, a formulation may be applied to the skin with the aid of a structure, such as a backing member, bandage or cover, for example a matrix patch.

There are several classes of topical pharmaceutical compositions. In some embodiments, the pharmaceutical compositions described herein are gels, lotions, creams, ointments, topical solutions, drops, jellies, balms, foams, mousses, patches, pastes, aerosols, sprays, powders, dispersible powders, granules, emulsions, sponges, tapes, tinctures or solids.

In some embodiments, the pharmaceutical compositions are gels, creams or ointments. In some embodiments, the pharmaceutical compositions are gels. In some embodiments, the pharmaceutical compositions are creams. In some embodiments, the pharmaceutical compositions are ointments. In some embodiments, the API is clofazimine, or a pharmaceutically acceptable salt thereof. In some embodiments, the API is one listed in Table 1, or a pharmaceutically acceptable salt thereof.

In some embodiments, the area to which the pharmaceutical composition is applied is washed prior to administration. In some embodiments, the area to which the pharmaceutical composition is applied is washed and dried prior to administration. In some embodiments, the area to which the pharmaceutical composition is applied is washed, dried and exfoliated prior to administration.

In some embodiments, a herein described pharmaceutical composition is administered once daily. In some embodiments, the pharmaceutical composition is administered twice daily. In some embodiments, the pharmaceutical composition is administered three times daily. In some embodiments, the pharmaceutical composition is administered four times daily. In some embodiments, the pharmaceutical composition is administered more than four times daily. In some embodiments, the pharmaceutical composition is administered no more than once daily. In some embodiments, the pharmaceutical composition is administered at least once daily. In some embodiments, the pharmaceutical composition is administered at least twice daily. In some embodiments, the pharmaceutical composition is administered one or more times a day for at least 1 day, 3 days, 7 days, 14 days, 21 days, 28 days, 1 month, 2 months, 3 months, 6 months, 12 months, 2 years, or any numbers or ranges in between, for example, 1, 2, 3, 4, 5, 6, 7, 8, 9, 10, 11, 12, 13, 14, 15, 16, 17, 18, 19, 20, 21, 22, 23, 24, 25, 26, 27, or 28 days, or 1, 2, 3, 4, 5, 6, 7, 8, 9, 10, 11, or 12 months. In some embodiments, the pharmaceutical composition is administered for at least a week, at least a month, at least 3 months, at least 6 months, or at least a year. In some embodiments, the pharmaceutical composition is administered for at most a month, at most 3 months, at most 6 months, at most a year, or at most 10 years. In some embodiments, the number of the daily application of the pharmaceutical composition depends upon the severity of the disease or disorder to be treated. In some embodiments, the pharmaceutical composition is administered as needed, i.e. in order to relieve symptoms of the disease or disorder.

In some embodiments, after administration of the composition to the topical area, the composition and area may be covered with a protective cloth, bandage, gauze, wrap or the like. The protective cloth, bandage, gauze or wrap may prevent transfer of the composition to, for example, clothing, furniture, bedding, third parties and the like. The protective cloth, bandage, gauze or wrap may protect the area from, for example, dirt, germs, bacteria and the like.

### Combinations

In some embodiments, pharmaceutical compositions described herein may further comprise a second API. In some embodiments, the second API is an anti-inflammatory, a topical anesthetic or a combination thereof. Typical topical anesthetics include, but are not limited to, lidocaine, xylocaine, buprenorphine and fentanyl. Any suitable topical corticosteroid can be employed as an anti-inflammatory drug. Suitable exemplary corticosteroids include cortisol (hydrocortisone); tetrahydrocortisol; prednisone (cortisone); prednisolone (cortisol); 6α-methylprednisolone; fludrocortisone (9α-fluorocortisol); 11-desoxycortisol; cortisone (11-dehydrocortisol); corticosterone; triamcinolone (9α-fluoro-16α-hydroxyprednisolone); paramethasone (6α-fluoro-16α-methylprednisolone); betamethasone (9α-fluoro-16β-methylprednisolone); dexamethasone (9α-fluoro-16α-methylprednisolone); desoxycorticosterone acetate (doca acetate, percorten acetate); desoxycorticosterone pivalate (percorten pivalate); fludrocortisone acetate (florine acetate); cortisol (hydrocortisone) (cortef, hydrocortone); cortisol acetate (cortef acetate, hydrocortone acetate); cortisol cypionate (cortef); cortisol sodium phosphate (hydrocortone phosphate); cortisol sodium succinate (solu-cortef); beclopmethasone dipropionate (vanceril); betamethasone (celestone); betamethasone sodium phosphate and acetate (celestone soluspan); betamethasone dipropionate (diprosone); betamethasone valerate (valisone); betamethasone benzoate (benisone, flurodate); cortisone acetate (cortone acetate); dexamethasone (decadron, gammacorten); dexamethasone sodium phosphate (decadron phosphate, hexadrol phosphate); dexamethasone acetate (decadron-L.A.); fuprednisolone (alphadrol); meprednisone (betapar); methylprednisolone (medrol); methylprednisolone acetate (depo-medrol, medrol acetate); methylprednisolone sodium succinate (solu-medrol); paramethasone acetate (haldrone); prednisolone (delta-cortef); prednisolone acetate (meticortelone acetate); prednisolone sodium phosphate (hydeltrasol); prednisolone sodium succinate (meticortelone soluble); prednisolone tebutate (hydelta-T.B.A.); prednisone (deltasone, paracort); triamcinolone (aristocort, kenacort); triamcinolone acetonide (aristoderm, kenalog); triamcinolone diacetate (aristocort diacetate, kienacort diacetate); triamcinolone hexacotonide (aristospan); desonide (tridesilon); desoximetasone (topicort); flumethasone pivalate (locorten); fluocinolone acetonide (fluonid, synalar); fluocinonide (lidex, topsyn); fluorometholone (oxylone); flurandrenolide (cordran); halcinonide (halog); and medrysone (HMS liquifilm, medrocort).

In some embodiments, the second API is an anti-pruritic agent. Anti-pruritic agents include, but are not limited to, pramoxine, diphenhydramine, benzocaine, lidocaine, bupivacaine, chloroprocaine, dibucaine, etidocaine, mepivacaine, tetracaine, dyclonine, hexylcaine, lignocaine, phenacaine, procaine, ketamine, phenol, butamben, butambenpicrate, cocaine, dimethisoquin, diperodon, dyclonine, methapyriline, oxyprocaine, p-buthylaminobenzoic acid 2-(die-ethylamino) ethyl ester, piperocaine, prilocaine, tripelennamine, dyclonine, resorcinol, cinchocaine, dexivacaine, diamocaine, levobupivacaine, oxethazaine, proparacaine, propoxycaine, pyrrocaine, risocaine, rodocaine, ropivacaine, pramocaine, proxazocain, 4-(3-(p-butoxyphenoxy)propyl) morpholine, gamma-morpholinopropyl 4-n-butoxyphenyl ether, p-butoxyphenyl gamma-morpholinopropyl ether, 4-[3-(4-butoxyphenoxy)-propyl]morpholine, menthol, camphor, menthyl anthranilate, and pharmaceutically acceptable salts thereof, derivatives, and mixtures thereof.

In some embodiments, the second API is an antimicrobial agent. Examples of antimicrobial agents include penicillins and related drugs, carbapenems, cephalosporins and related drugs, erythromycin, aminoglycosides, bacitracin, gramicidin, mupirocin, chloramphenicol, thiamphenicol, fusidate sodium, lincomycin, clindamycin, macrolides, novobiocin, polymyxins, rifamycins, spectinomysin, tetracyclines, vanomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, eveminomycin, glycopeptide, glyclyclycline, ketolides, oxazolidinone; imipenen, amikacin, netilmicin, fosfomycin, gentamycin, ceftriaxone, Ziracin, Linezolid, Synercid, Aztreonam, and Metronidazole, Epiroprim, Sanfetrinem sodium, Biapenem, Dynemicin, Cefluprenam, Cefoselis, Sanfetrinem celexetil, Cefpirome, Mersacidin, Rifalazil, Kosan, Lenapenem, Veneprim, Sulopenem, ritipenam acoxyl, Cyclothialidine, micacocidin A, carumonam, Cefozopran and Cefetamet pivoxil.

In some embodiments, the second API is an anti-acne agent. Examples of topical anti-acne agents include adapalene, azelaic acid, benzoyl peroxide, clindamycin and clindamycin phosphate, doxycycline, erythromycin, keratolytics such as salicylic acid and retinoic acid (Retin-A), norgestimate, organic peroxides, retinoids such as isotretinoin and tretinoin, sulfacetamide sodium, and tazarotene. Particular anti-acne agents include adapalene, azelaic acid, benzoyl peroxide, clindamycin (e.g., clindamycin phosphate), doxycycline (e.g., doxycycline monohydrate), erythromycin, isotretinoin, norgestimate, sulfacetamide sodium, tazarotene, etretinate and acetretin.

In some embodiments, the second API is an antihistamine. Examples of antihistamines include diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride, and the like. Examples of local anesthetic agents include dibucaine hydrochloride, dibucaine, lidocaine hydrochloride, lidocaine, benzocaine, p-buthylaminobenzoic acid 2-(die-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine and dyclonine hydrochloride.

In some embodiments, the second API is an antiseptic. Examples of antiseptics include alcohols, quaternary ammonium compounds, boric acid, chlorhexidine and chlorhexidine derivatives, iodine, phenols, terpenes, bactericides, disinfectants including thimerosal, phenol, thymol, benzalkonium chloride, benzethonium chloride, chlorhexidine, povidone iode, cetylpyridinium chloride, eugenol and trimethylammonium bromide.

In some embodiments, the second API is an analgesic. Examples of analgesics include alfentanil, benzocaine, buprenorphine, butorphanol, butamben, capsaicin, clonidine, codeine, dibucaine, enkephalin, fentanyl, hydrocodone, hydromorphone, indomethacin, lidocaine, levorphanol, meperidine, methadone, morphine, nicomorphine, opium, oxybuprocaine, oxycodone, oxymorphone, pentazocine, pramoxine, proparacaine, propoxyphene, proxymetacaine, sufentanil, tetracaine and tramadol.

In some embodiments, the second API is an anesthetic. Examples of anesthetic agents include alcohols such as phenol; benzyl benzoate; calamine; chloroxylenol; dyclonine; ketamine; menthol; pramoxine; resorcinol; troclosan; procaine drugs such as benzocaine, bupivacaine, chloroprocaine; cinchocaine; cocaine; dexivacaine; diamocaine; dibucaine; etidocaine; hexylcaine; levobupivacaine; lidocaine; mepivacaine; oxethazaine; prilocaine; procaine; proparacaine; propoxycaine; pyrrocaine; risocaine; rodocaine; ropivacaine; tetracaine; and derivatives, such as pharmaceutically acceptable salts and esters including bupivacaine HCl, chloroprocaine HCl, diamocaine cyclamate, dibucaine HCl, dyclonine HCl, etidocaine HCl, levobupivacaine HCl, lidocaine HCl, mepivacaine HCl, pramoxine HCl, prilocaine HCl, procaine HCl, proparacaine HCl, propoxycaine HCl, ropivacaine HCl, and tetracaine HCl.

In some embodiments, the second API is an antihemorrhagic agent. Examples of antihemorrhagic agents include thrombin, phytonadione, protamine sulfate, aminocaproic acid, tranexamic acid, carbazochrome, carbaxochrome sodium sulfanate, rutin and hesperidin.

The amount a suitable second API can be present in, is about 0.1 wt % to about 99.9 wt % of the composition. Typically, the amount of a second API will depend upon the specific agent employed in the composition. In some embodiments, the API can be up to about 10 wt %, up to about 5 wt %, up to about 2 wt %, up to about 1 wt % or up to about 0.1 wt % of the composition.

### Doses

Dosing can be dependent on severity and responsiveness of the disease or condition to be treated, with the course of treatment lasting from several days to several months, or until a cure is effected or a diminution of disease state or a diminution of disease symptoms is achieved. Optimal dosing schedules may be calculated from measurements of drug accumulation in the body of the patient. Persons of skill in the medical arts can determine optimum dosages, dosing methodologies and repetition rates. Dosage levels on the order of from about 1 ug/kg to about 100 mg/kg of body weight per administration are useful in the treatment of a disease. In some embodiments, dosage amounts are from about 0.001 mg/kg to about 1,000 mg/kg. In some embodiments, dosage amounts are from about 0.001 mg/kg to about 0.01 mg/kg, about 0.001 mg/kg to about 0.1 mg/kg, about 0.001 mg/kg to about 0.25 mg/kg, about 0.001 mg/kg to about 0.5 mg/kg, about 0.001 mg/kg to about 0.75 mg/kg, about 0.001 mg/kg to about 1 mg/kg, about 0.001 mg/kg to about 5 mg/kg, about 0.001 mg/kg to about 10 mg/kg, about 0.001 mg/kg to about 50 mg/kg, about 0.001 mg/kg to about 100 mg/kg, about 0.001 mg/kg to about 1,000 mg/kg, about 0.01 mg/kg to about 0.1 mg/kg, about 0.01 mg/kg to about 0.25 mg/kg, about 0.01 mg/kg to about 0.5 mg/kg, about 0.01 mg/kg to about 0.75 mg/kg, about 0.01 mg/kg to about 1 mg/kg, about 0.01 mg/kg to about 5 mg/kg, about 0.01 mg/kg to about 10 mg/kg, about 0.01 mg/kg to about 50 mg/kg, about 0.01 mg/kg to about 100 mg/kg, about 0.01 mg/kg to about 1,000 mg/kg, about 0.1 mg/kg to about 0.25 mg/kg, about 0.1 mg/kg to about 0.5 mg/kg, about 0.1 mg/kg to about 0.75 mg/kg, about 0.1 mg/kg to about 1 mg/kg, about 0.1 mg/kg to about 5 mg/kg, about 0.1 mg/kg to about 10 mg/kg, about 0.1 mg/kg to about 50 mg/kg, about 0.1 mg/kg to about 100 mg/kg, about 0.1 mg/kg to about 1,000 mg/kg, about 0.25 mg/kg to about 0.5 mg/kg, about 0.25 mg/kg to about 0.75 mg/kg, about 0.25 mg/kg to about 1 mg/kg, about 0.25 mg/kg to about 5 mg/kg, about 0.25 mg/kg to about 10 mg/kg, about 0.25 mg/kg to about 50 mg/kg, about 0.25 mg/kg to about 100 mg/kg, about 0.25 mg/kg to about 1,000 mg/kg, about 0.5 mg/kg to about 0.75 mg/kg, about 0.5 mg/kg to about 1 mg/kg, about 0.5 mg/kg to about 5 mg/kg, about 0.5 mg/kg to about 10 mg/kg, about 0.5 mg/kg to about 50 mg/kg, about 0.5 mg/kg to about 100 mg/kg, about 0.5 mg/kg to about 1,000 mg/kg, about 0.75 mg/kg to about 1 mg/kg, about 0.75 mg/kg to about 5 mg/kg, about 0.75 mg/kg to about 10 mg/kg, about 0.75 mg/kg to about 50 mg/kg, about 0.75 mg/kg to about 100 mg/kg, about 0.75 mg/kg to about 1,000 mg/kg, about 1 mg/kg to about 5 mg/kg, about 1 mg/kg to about 10 mg/kg, about 1 mg/kg to about 50 mg/kg, about 1 mg/kg to about 100 mg/kg, about 1 mg/kg to about 1,000 mg/kg, about 5 mg/kg to about 10 mg/kg, about 5 mg/kg to about 50 mg/kg, about 5 mg/kg to about 100 mg/kg, about 5 mg/kg to about 1,000 mg/kg, about 10 mg/kg to about 50 mg/kg, about 10 mg/kg to about 100 mg/kg, about 10 mg/kg to about 1,000 mg/kg, about 50 mg/kg to about 100 mg/kg, about 50 mg/kg to about 1,000 mg/kg, or about 100 mg/kg to about 1,000 mg/kg. In some embodiments, dosage amounts are from about 0.001 mg/kg, about 0.01 mg/kg, about 0.1 mg/kg, about 0.25 mg/kg, about 0.5 mg/kg, about 0.75 mg/kg, about 1 mg/kg, about 5 mg/kg, about 10 mg/kg, about 50 mg/kg, about 100 mg/kg, or about 1,000 mg/kg. In some embodiments, dosage amounts are at least about 0.001 mg/kg, about 0.01 mg/kg, about 0.1 mg/kg, about 0.25 mg/kg, about 0.5 mg/kg, about 0.75 mg/kg, about 1 mg/kg, about 5 mg/kg, about 10 mg/kg, about 50 mg/kg, or about 100 mg/kg. In some embodiments, dosage amounts are at most about 0.01 mg/kg, about 0.1 mg/kg, about 0.25 mg/kg, about 0.5 mg/kg, about 0.75 mg/kg, about 1 mg/kg, about 5 mg/kg, about 10 mg/kg, about 50 mg/kg, about 100 mg/kg, or about 1,000 mg/kg. In some embodiments, the dosage amount is based on the amount of an API or a pharmaceutically acceptable salt thereof (such as clofazimine) over the weight of the subject. In some embodiments, the dosage amount is based on a herein described pharmaceutical composition over the weight of the subject.

Certain factors may influence the dosage required to effectively treat a subject, including, but not limited to, the severity of the disease or disorder, previous treatments, the general health and/or age of the subject, and other diseases present. The effective dosage for treatment may increase or decrease over the course of a particular treatment. Changes in dosage may result and become apparent from the results of diagnostic assays. For example, a subject may be monitored after administering a composition. Based on information from the monitoring, an increased or decreased amount of the composition can be administered.

### Conditions

In some embodiments, pharmaceutical compositions described herein are used to treat diseases or disorders. In some embodiments, pharmaceutical compositions described herein are used to treat diseases or disorders of a human. In some embodiments, the diseases are inflammatory diseases or disorders. In some embodiments, the diseases are inflammation-mediated diseases or disorders. In some embodiments, the diseases or disorders are skin diseases or disorders. In some embodiments, the diseases or disorders are inflammatory skin diseases or disorders. In some embodiments, the diseases or disorders are dermatological diseases or disorders. In some embodiments, the diseases or disorders are inflammatory dermatological diseases or disorders. In some embodiments, the diseases or disorders are associated with an over-expression of Kv1.3 channels, such as, for example, breast and lung cancer, melanoma, or chronic lymphocytic leukaemia. In some embodiments, the diseases or disorders are associated with an over-secretion of IL-2. Accordingly, in one aspect, described here are methods of treating a disease or condition associated with an over-expression of Kv1.3 channels, such as a cancer, using a topical formulation described herein. In one aspect, described here are methods of inhibiting Kv1.3 channels by administering a topical formulation described herein. In one aspect, described here are methods of inhibiting the secretion or over-secretion of IL-2 by administering a topical formulation described herein.

In some embodiments, the dermatological disease or skin disorder is rosacea, eczema (i.e., atopic dermatitis), acne, hidradenitis suppurativa, Palmo-Plantar Pustulosis, Psoriasis, Pustular Psoriasis, Generalized Pustular Psoriasis, Pyoderma Gangrenosum, Erosive Pustular Dermatosis of the Scalp, Sweet's Syndrome, Bowel-associated Dermatosis-arthritis Syndrome, Acute Generalized Exanthematous Pustulosis, Keratoderma Blenorrhagicum, Sneddon-Wilkinson Disease, IgA Pemphigus, Amicrobial Pustulosis of the Folds, Infantile Acropustulosis, Transient Neonatal Pustulosis, Neutrophilic Eccrine Hidradenitis, Rheumatoid Neutrophilic Dermatitis, Neutrophilic Urticaria, Dermatitis Herpetiformis, Linear IgA disease (LAD), Inflammatory Epidermolysis Bullosa Aquisita, Alopecia Areata, Autoimmune Angioedema, Autoimmune progesterone dermatitis, Autoimmune urticaria, Bullous pemphigoid, Cicatricial pemphigoid, Dermatitis herpetiformis, Epidermolysis bullosa acquisita, Erythema nodosum, Gestational pemphigoid, Lichen planus, Lichen sclerosus, Morphea, Pemphigus vulgaris, Pityriasis lichenoides et varioliformis acuta, Mucha-Habermann disease, Vitiligo, or Neutrophilic Dermatosis of the Dorsal Hands, Basal Cell Carcinoma, Bowen's Disease, Congenital Erythropoietic Porphyria, Contact Dermatitis, Darier's disease, Disseminated Superficial Actinic Porokeratosis, Dystrophic Epidermolysis Bullosa, Eczema, Epidermolysis bullosa simplex, Extra-mammary Paget's Disease, Erythropoietic Protoporphyria & X-Linked Dominant Protoporphyria, Fungal infections, Hailey-Hailey Disease, Herpes simplex, Hidradenitis Suppurativa, Hirsutism, Hyperhidrosis, Ichthyosis, Impetigo, Keloids, Keratosis pilaris, Lichen planus, Lichen Sclerosus, Melanoma, Melasma, Mucous Membrane Pemphigoid, Pemphigoid, Pemphigus Vulgaris, Pityriasis Lichenoides, Pityriasis Rubra Pilaris, Plantar Warts (Verrucas), Polymorphic Light Eruption, Pyoderma Gangrenosum, Scabies, Shingles (herpes zoster), Squamous Cell Carcinoma, Sweet's Syndrome, Urticaria and Angioedema or any combination thereof.

In some embodiments, the pharmaceutical compositions described herein are used to treat psoriasis, keratosis, eczema, rosacea, acne vulgaris, dermatitis, pruritus, seborrhea, skin cancers, inflammation, other skin disorders responsive to acitretin or etretinate, and combinations thereof. In some embodiments, the pharmaceutical compositions described herein are used to treat psoriasis, vitiligo, lupus erythematosus, eczema, chronic eczema, dermatitis and contact dermatitis.

In some embodiments, the pharmaceutical compositions described herein are used to treat psoriasis. In some embodiments, the psoriasis is plaque psoriasis, guttate psoriasis, inverse psoriasis, pustular psoriasis, erythrodermic psoriasis, or any combination thereof. In some embodiments, the psoriasis is mild. In some embodiments, the psoriasis is moderate. In some embodiments, the psoriasis is severe. Skin disorders vary greatly in symptoms and severity. They can be temporary or permanent, and may be painless or painful. Some have situational causes, while others may be genetic. Some skin conditions are minor, while others can be life-threatening.

In one aspect, described herein are methods of treating diseases or disorders in a subject in need thereof. In some embodiments, pharmaceutical compositions described herein are administered to the skin of the subject, e.g., scalp, face, elbows, knees, arms, legs, back such as lower back, etc. In some embodiments, the subject is a mammal, such as a pet (e.g. dog, cat, pig) or human. In some embodiments, the subject is a human. In some embodiments, the human is 10 years old or less. In some embodiments, the human is 21 years old or less. In some embodiments, the human is 21 years old or more. In some embodiments, the human is 30 years old or more. In some embodiments, the human is 40 years old or more. In some embodiments, the human is 50 years old or more. In some embodiments, the subject has a skin lesion area of about 2-10% of body surface area. In some embodiments, the subject has a skin lesion area of about 1-20% of body surface area. In some embodiments, the subject has a skin lesion area of about 1-40%, 5-20%, 5-30%, 10-20%, 2-15%, 2-10% or 1-20% of body surface area. In some embodiments, the subject has a skin lesion area of at least about 1%, 2%, 5%, 10%, 15%, 20%, 25%, or 30% of body surface area. In some embodiments, the subject has a skin lesion area of at most about 5%, 7.5%, 10%, 15%, 20%, 25%, 30%, 40%, 50%, or 80% of body surface area.

### EXAMPLES

The following examples are used to illustrate the present disclosure and shall not be considered to limit the scope of the present disclosure. The terms Transcutol, 2-(2-Ethoxyethoxy)ethanol, diethylene glycol monoethyl ether are recognized in the art and used interchangeably herein to represent a compound of formula:

EtOCH₂CH₂OCH2CH₂OH.

### Example 1: Solubility of Clofazimine in Aqueous Media

Clofazimine was added to various aqueous media, at various pH values (2-5 mL) in a 10 mL glass vial, at 25° C., with stirring. When no further dissolution was observable, the suspension was stirred for a further 24 hours at 25° C. If it appeared the API was still dissolving, the suspension was stirred for a further 24 hours at 25° C. This cycle was repeated until no further dissolution was observable. The suspension was then centrifuged and the supernatant filtered by microfiltration. The filtrate was diluted and clofazimine content determined by HPLC, and the solubility recorded and presented in Table 5.

TABLE 5

| Medium | Solubility (mg/ml) |
|---|---|
| pH 3.0 | 0.00 |
| pH 4.0 | 0.00 |
| pH 5.0 | 0.00 |
| pH 6.0 | 0.00 |
| pH 7.4 | 0.00 |
| pH 4.5 + 0.25% SDS | 0.71 |

### Example 2: Solubility of Clofazimine in Fatty Acids

Clofazimine was dissolved in fatty acids, at 25° C., and the solubility recorded and presented in Table 6

TABLE 6

| Solvent | Chemical Structure | Solubility (mg/ml) |
|---|---|---|
| Acetic acid | CH₃COOH | 30.19 |
| Propionic acid | CH₃CH₂COOH | 28.24 |
| Butyric acid | CH₃(CH₂)₂COOOH | 49.22 |
| Hexanoic acid | CH₃(CH₂)₄COOOH | 80.66 |
| Heptanoic acid | CH₃(CH₂)₅COOOH | 78.28 |
| Octanoic acid | CH₃(CH₂)₆COOOH | 56.33 |
| Isovaleric acid | CH₃CH(CH₃)CH₂COOH | 58.62 |
| Oleic acid | CH₃(CH₂)₇CH═CH(CH₂)₇COOH | 105.70 |
| Linoleic acid | CH₃(CH₂)₄CH═CH(CH₂)CH═CH(CH₂)₇COOH | 90.22 |

### Example 3: Solubility of Clofazimine in Various Solvents

Clofazimine was added to various solvents (2-5 mL) in a 10 mL glass vial, at 25° C., with stirring. When no further dissolution was observable, the suspension was stirred for a further 24 hours at 25° C. If it appeared the API was still dissolving, the suspension was stirred for a further 24 hours at 25° C. This cycle was repeated until no further dissolution was observable. The suspension was then centrifuged and the supernatant filtered by microfiltration. The filtrate was diluted and clofazimine content determined by HPLC, and the solubility recorded and presented in Table 7.

TABLE 7

| Solvent | Solubility (mg/ml) |
|---|---|
| Glycerol | 0.49 |
| Propane 1,2-diol | 0.42 |
| Ethanol | 0.95 |
| Polyglycerol fatty acid ester | 4.96 |
| Corn oil | 5.80 |
| Soybean oil | 5.10 |
| Coconut oil | 5.10 |
| Octanol | 7.48 |
| Tween 20 | 7.40 |
| Tween 80 | 10.07 |
| PEG 400 | 9.51 |
| Isopropyl myristate | 8.51 |
| Glyceryl monooleate | 8.86 |
| Polyoxyethylene hydrogenated castor oil | 8.67 |
| Castor oil | 11.03 |
| Oleic acid polyethylene glycol glyceride | 12.20 |
| Transcutol | 21.55 |
| Labrasol | 25.38 |
| Laurocapram | 46.72 |

### Example 4: Skin Permeability of Clofazimine Preparations

The permeability of clofazimine preparations, as described in Examples 5-14, in mouse skin was evaluated.

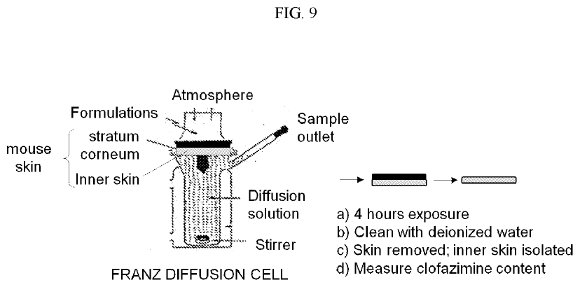

Experiments were conducted in independent vertical Franz diffusion cells at 32° C.±0.5° C. at a rotation speed of 100 rpm. The acceptor compartment volume was 7 mL, with a diffusion area of 3.14 cm². The acceptor compartment was filled with physiological saline solution. NU/NU nude mice skin was mounted, with the inner surface of the skin facing the saline diffusion solution (ensuring there were no air bubbles) and the stratum corneum exposed to the atmosphere. Clofazimine test preparation (0.5 g) was applied onto the skin and exposed for 4 hours, after which time the skin was removed from the device and cleaned by wiping with ethanol. The inner skin layer was isolated and the stratum corneum discarded. The inner skin later was cut into small pieces, transferred to a 100 mL volumetric flask and extracted by immersion in acetonitrile for 24 hours. The clofazimine content in the acetonitrile extract was determined by HPLC, and the clofazimine content per unit area of epidermis calculated. FIG. 9 shows a cartoon representation of the apparatus used for this procedure. This procedure was used to evaluate the skin permeability of the clofazimine preparations described in Examples 5-14.

### Example 5: Skin Permeability of Clofazimine Solutions

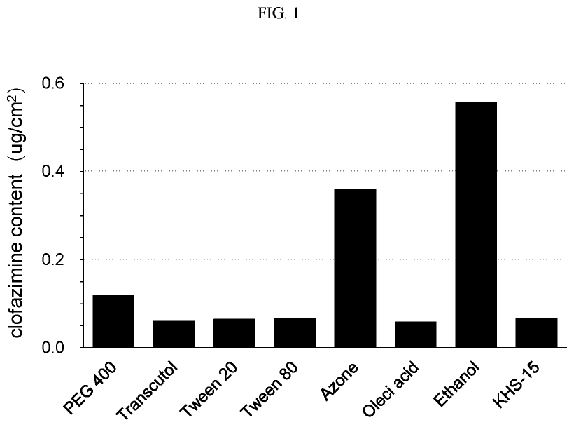

The permeability of clofazimine solutions in mouse skin was evaluated. Clofazimine (100 mg) was dissolved in eight different organic solvents (99.9 g); PEG 400, Transcutol, Tween 20, Tween 80, Laurocapram (N-Dodecyclcaprolactam), oleic acid, ethanol, and polyoxyethylene 15 hydroxystearic acid (KHS-15). The skin permeability of the various clofazimine solutions was examined in the NU/NU nude mice model, as described in example 4, and the results are shown in Table 8 and graphically in FIG. 1.

TABLE 8

| | clofazimine content |
|---|---|
| Solvent | (ug/cm²) |
| Laurocapram | 0.3593 |
| PEG 400 | 0.1174 |
| Transuctol | 0.0596 |
| Tween 20 | 0.0639 |
| Tween 80 | 0.0660 |
| Oleic acid | 0.0577 |
| Ethanol | 0.5565 |
| KHS-15 | 0.0660 |

### Example 6: Skin Permeability of Clofazimine Gel Preparations

The permeability of clofazimine gel preparations in mouse skin was evaluated. Clofazimine (100 mg; 0.1%, wt) was dissolved in various different organic solvents (9.9 g): Transuctol, PEG 400, Labrasol (or PEG-8 caprylic/capric glycerides), and Tween 80. A gel containing Carbomer 940 (0.5 g; 0.5 wt %) in water was prepared, and the pH adjusted to 7.0 by addition of triethanolamine (TEA). The clofazimine solution was added to the carbomer gel, with stirring until even dispersion was achieved; see Table 9 for amounts (Propylene glycol and Laurocapram were also evaluated, but resulted in clofazimine precipitation and formation of separated layers, respectively)

TABLE 9

| Component | Content (wt %) |
|---|---|
| Clofazimine | 0.1 |
| Carbomer 940 | 0.5 |
| Triethanolamine | Adjusted to pH 7.0 |
| Solvent | 10 |
| Water | Added to 100% |

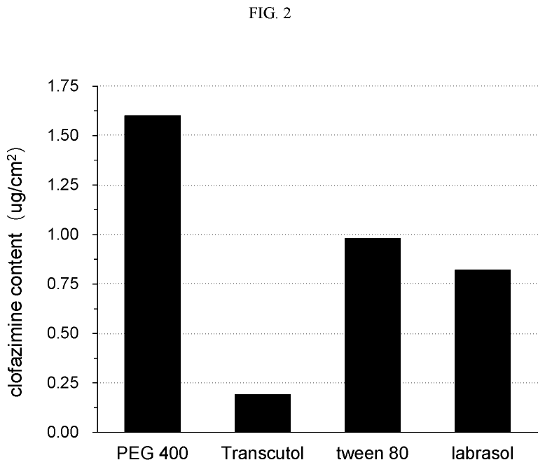

The skin permeability of the various clofazimine gel preparations was examined in the NU/NU nude mice model, as described in Example 4, and the results are shown in Table 10 and graphically in FIG. 2.

TABLE 10

| | clofazimine content |
|---|---|
| Solvent | (ug/cm²) |
| PEG 400 | 1.6000 |
| Transuctol | 0.1894 |
| Tween 80 | 0.9800 |
| labrasol | 0.8200 |

### Example 7: Comparison of Skin Permeability of Clofazimine Solutions Vs. Gel Preparations

The mouse skin permeability of various clofazimine formulations, as described in either Example 5 (solution) or 6 (gel preparation), and as shown in Table 11, were compared.

TABLE 11

| Component | | Solution | | | Gel | |
|---|---|---|---|---|---|---|
| Clofazimine | 0.1 | 0.1 | 0.1 | 0.1 | 0.1 | 0.1 |
| Carbomer 940 | — | — | — | 0.5 | 0.5 | 0.5 |
| Triethanolamine | — | — | — | pH = 7.0 | pH = 7.0 | pH = 7.0 |
| Solvent | Labrasol | Tween 80 | PEG400 | Labrasol | Tween 80 | PEG400 |
| Amount | 99.9 | 99.9 | 99.9 | 10 | 10 | 10 |
| Water | — | — | — | to 100% | to 100% | to 100% |

### Amounts as wt %

*Adjust to pH=7

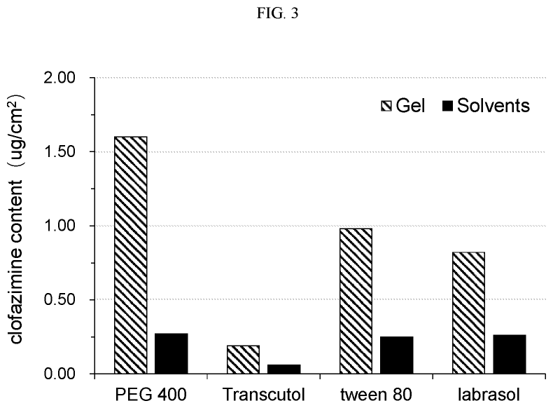

The skin permeability of the clofazimine solutions and gels was examined in the NU/NU nude mice model, as described in Example 4, and the results are shown in Table 12 and graphically in FIG. 3.

TABLE 12

| Preparation | PEG 400 | Transcutol | Tween 80 | labrasol |
|---|---|---|---|---|
| Gel | 1.6000 | 0.1894 | 0.9800 | 0.8200 |
| Solvents | 0.2700 | 0.0596 | 0.2500 | 0.2600 |

### Example 8: Effect of Penetration Enhancers on Skin Permeability of Clofazimine Gel Preparations

Clofazimine (100 mg) was dissolved in in Transcutol (9.9 g). This solution was added to a solution of carbomer (0.5 g) in water. A penetration enhancer (4 g) was then added, and the pH adjusted to 7.0 by addition of triethanolamine. The mixture was stirred for even dispersion until a gel formed. The penetration enhancers examined were: 0.05% sodium dodecyl sulfate (SDS), ethanol, DMSO, Tween 80, Tween 20, n-octanol, oleic acid, and lecithin (containing 20-30% of phosphatidylcholine), and water (as a control). The gel formulations are summarized in Table 13.

TABLE 13

| Component | Content (wt %) |
|---|---|
| Clofazimine | 0.1 |
| Carbomer 940 | 0.5 |
| Triethanolamine | adjust pH = 7.0 |
| Transcutol | 20 |
| Penetration enhancer | 4 |
| Water | to 100% |

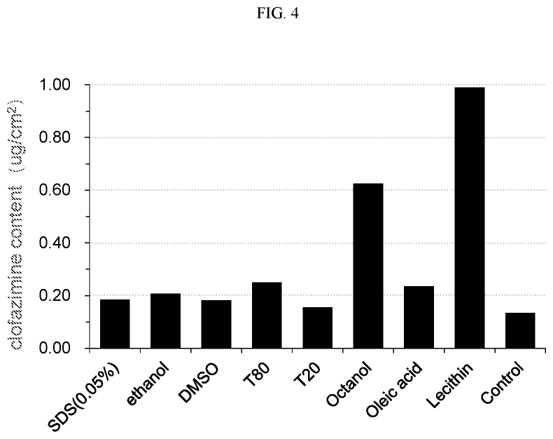

The skin permeability of the various clofazimine gel preparations was examined in the NU/NU nude mice model, as described in Example 4, and the results are shown in Table 14 and graphically in FIG. 4.

TABLE 14

| Penetration | clofazimine content |
|---|---|
| Enhancer | (ug/cm²) |
| SDS (0.05%) | 0.1829 |
| Ethanol | 0.2055 |
| DMSO | 0.1806 |
| Tween 80 | 0.2484 |
| Tween 20 | 0.1535 |
| Octanol | 0.6230 |
| Oleic acid | 0.2344 |
| Lecithin | 0.9881 |
| Control | 0.1331 |

### Example 9: Effect of Lecithin Concentration on Skin Permeability of Clofazimine Gel Preparations

Clofazimine (100 mg) was dissolved in Transcutol (20 g). This solution was added to a solution of carbomer (0.5) in water. Various amounts of lecithin (20-30% containing phosphatidylcholine) were then added and the pH adjusted to 7.0 by addition of triethanolamine. The mixture was stirred for even dispersion until a gel formed. The gel formulations are summarized in Table 15.

TABLE 15

| Component | Content (wt %) |
|---|---|
| Clofazimine | 0.1 |
| Carbomer 940 | 0.5 |
| Triethanolamine | Adjust to pH = 7.0 |
| Transcutol | 20 |
| Lecithin | 2-19 |
| Water | to 100% |

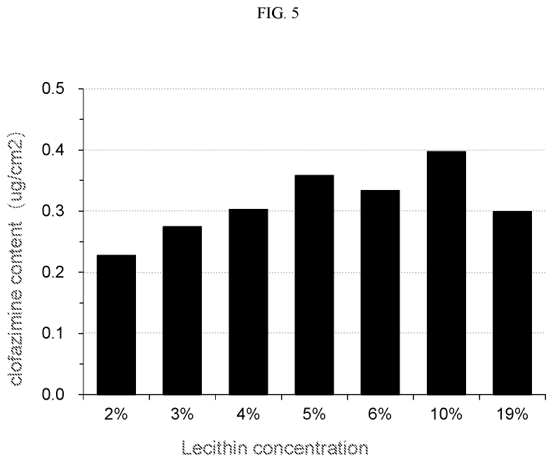

The skin permeability of the various clofazimine solutions was examined in the NU/NU nude mice model, as described in Example 4, and the results are shown in Table 16 and graphically in FIG. 5.

TABLE 16

| Amount of | clofazimine content |
|---|---|
| lecithin (wt %) | (ug/cm²) |
| 2% | 0.2272 |
| 3% | 0.2745 |
| 4% | 0.3026 |
| 5% | 0.3578 |
| 6% | 0.3335 |
| 10% | 0.3968 |
| 19% | 0.2989 |

### Example 10: Effect of pH on Skin Permeability of Clofazimine Hydrogel Preparations

Clofazimine (100 mg) was dissolved in Transcutol (20 g) and added to a solution of carbomer (0.5 g) in water. Lecithin (20-30% containing phosphatidylcholine; 5 g) was added, and the pH adjusted to 6.0, 7.0, 8.0, 9.0 or 9.5, by addition of triethanolamine. The mixture was stirred for even dispersion until a gel formed. The gel formulations are summarized in Table 17.

TABLE 17

| Component | Content (wt %) |
|---|---|
| Clofazimine | 0.1 |
| Carbomer 940 | 0.5 |
| Triethanolamine | Adjust to pH = 6.0-9.5 |
| Transcutol | 20 |
| Lecithin | 5 |
| Water | to 100% |

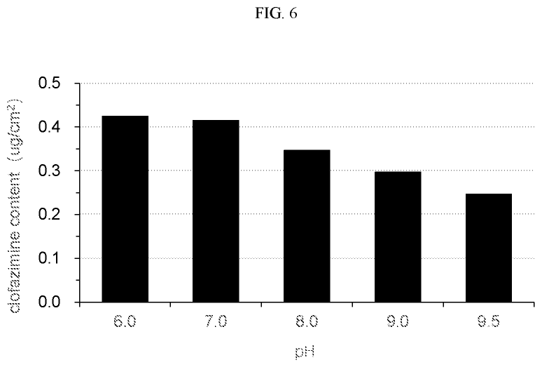

The skin permeability of the various clofazimine solutions was examined in the NU/NU nude mice model, as described in Example 4, and the results are shown in Table 18 and graphically in FIG. 6.

TABLE 18

| | clofazimine content |
|---|---|
| pH | (ug/cm²) |
| 6.0 | 0.4246 |
| 7.0 | 0.4146 |
| 8.0 | 0.3463 |
| 9.0 | 0.2970 |
| 9.5 | 0.2463 |

### Example 11: Effect of Clofazimine Concentration on Permeability of Clofazimine Gel Preparations

Various amounts of clofazimine were dissolved in Transcutol (20 g) and added to a solution of carbomer (0.5 g) in water. Lecithin (20-30% containing phosphatidylcholine; 5 g) was added, and the pH adjusted to 6.0, 7.0, 8.0, 9.0 or 9.5, by addition of triethanolamine. The mixture was stirred for even dispersion until a gel formed. The gel formulations are summarized in Table 19.

TABLE 19

| Component | Content (wt %) |
|---|---|
| Clofazimine | 0.0-0.5 |
| Carbomer 940 | 0.5 |
| Triethanolamine | Adjust pH = 7.0 |
| Transcutol | 20 |
| Lecithin | 5 |
| Water | Adjust to 100 |

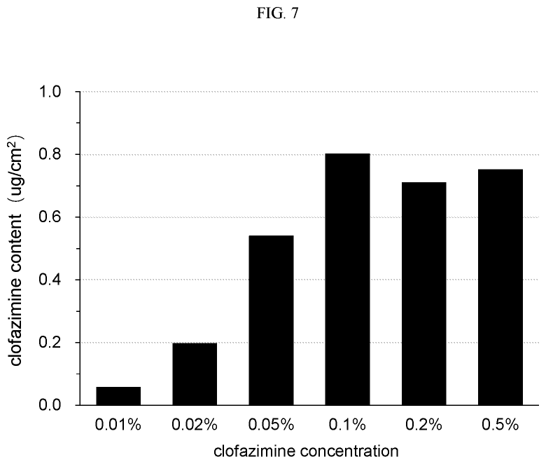

The skin permeability of the various clofazimine solutions was examined in the NU/NU nude mice model, as described in Example 4, and the results are shown in Table 20 and graphically in FIG. 7.

TABLE 20

| clofazimine | clofazimine content |
|---|---|
| amount | (ug/cm²) |
| 0.01% | 0.0568 |
| 0.02% | 0.1971 |
| 0.05% | 0.5394 |
| 0.1% | 0.8014 |
| 0.2% | 0.7102 |
| 0.5% | 0.7512 |

### Example 12: Effect of Carbomer Concentration on Permeability of Clofazimine Gel Preparations

Clofazimine (100 mg) was dissolved in Transcutol (20 g) and added to a solution of various amounts of carbomer (0.5, 0.8 and 1.0%) in water. Lecithin (20-30% containing phosphatidylcholine; 5 g) was added, and the pH adjusted to 7.0 by addition of triethanolamine. The mixture was stirred for even dispersion until a gel formed. The gel formulations are summarized in Table 21.

TABLE 21

| Component | Content (wt %) |
|---|---|
| Clofazimine | 0.1 |
| Carbomer 940 | 0.5-1.0 |
| Triethanolamine | Adjust pH = 7.0 |
| Transcutol | 20 |
| Lecithin | 5 |
| Water | Adjust to 100 |

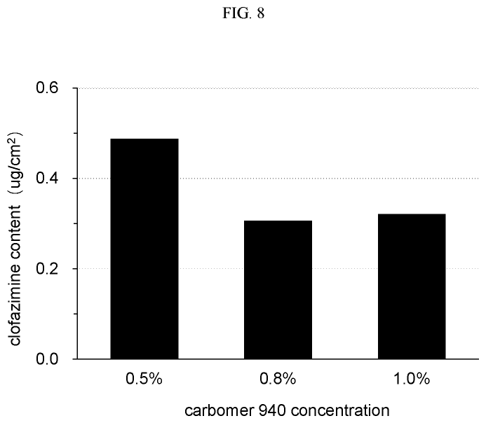

The skin permeability of the various clofazimine solutions was examined in the NU/NU nude mice model, as described in Example 4, and the results are shown in Table 22 and graphically in FIG. 8.

TABLE 22

| Carbomer 940 amount | clofazimine content (ug/cm²) |
|---|---|
| 0.5% | 0.4871 |
| 0.8% | 0.3054 |
| 1.0% | 0.3205 |

### Example 13: Clofazimine Gel Preparations

Three clofazimine gels containing a humectant (as a preservative, such as, but not limited to propylparaben and methylparaben) and an antioxidant (to prevent oxidation, such as, but not limited to butylated hydroxytoluene.)

Clofazimine was dissolved in Transcutol, and added to a mixture of Tween 80, propylene glycol, glycerin, allantoin, methylparaben, propylparaben and butylated hydroxytoluene, which was evenly stirred for full dispersion. The pH was adjusted to 6-7 by addition of triethanolamine to form a dark red transparent hydrogel. Table 23 provides the amounts for each preparation.

TABLE 23

| Component | F12-1 | F12-2 | F12-3 |
|---|---|---|---|
| Clofazimine | 0.005 | 0.1 | 1 |
| Transcutol | 10 | 20 | 30 |
| Carbomer | 0.5 | 0.5 | 0.5 |
| Tween 80 | 4 | 4 | 4 |
| Propylene glycol | 10 | 10 | 10 |
| Glycerin | 5 | 5 | 5 |
| Triethanolamine | adjust pH 7 | adjust pH 7 | adjust pH 7 |
| Allantoin | 0.5 | 0.5 | 0.5 |
| Methylparaben | 0.03 | 0.03 | 0.03 |
| Propylparaben | 0.01 | 0.01 | 0.01 |
| Butylated hydroxytoluene | 0.05 | 0.05 | 0.05 |
| Water | remainder | remainder | remainder |
| Total | 100% | 100% | 100% |

### Example 14: Clofazimine Cream Preparations

Three clofazimine creams were prepared.

Clofazimine and lecithin were dissolved in Transcutol, and heated to 80° C. A mixture of petrolatum, liquid paraffin and glyceryl monostearate were added and stirred until completely dissolved. A solution of sodium lauryl sulfate in an appropriate amount of water was heated to 85° C. until full dissolution. Methylparaben, propylparaben and BHT (amounts) were dissolved in propylene glycol and then added to the sodium lauryl sulfate solution, with stirring until full dissolution. This mixture was then added, with stirring, to the clofazimine solution, while maintaining the temperature at 85° C. After stirring evenly, the temperature is gradually lowered to room temperature, to obtain the desired cream. Table 24 provides the amounts for each preparation.

TABLE 24

| Component | F13-1 | F13-2 | F13-3 |
|---|---|---|---|
| Clofazimine | 0.1 | 0.1 | 0.1 |
| Octadecanol | 3.5 | 3.5 | 3.5 |
| Vaseline | 3 | 3 | 3 |
| Liquid paraffin | 5 | 5 | 5 |
| Triethanolamine | adjust pH 7 | adjust pH 7 | adjust pH 7 |
| Glyceryl monostearate | 3 | 3 | 3 |
| SDS | 1 | 1 | 0.5 |
| Transcutol | 10 | / | 20 |
| PEG 400 | / | 10 | |
| Lecithin | 5 | 5 | 5 |
| Propylene glycol | 15 | 15 | 15 |
| Glycerin | 5 | 5 | 5 |
| Allantoin | 0.5 | 0.5 | 0.5 |
| Methylparaben | 0.03 | 0.03 | 0.03 |
| Propylparaben | 0.01 | 0.01 | 0.01 |
| Butylated hydroxytoluene | 0.05 | 0.05 | 0.05 |
| Water | remainder | remainder | remainder |
| Total | 100% | 100% | 100% |

### Example 15: Compatibility of Clofazimine and Excipients

Clofazimine was dissolved into different excipients with different mass ratios, then the mixture was placed in the condition of 60° C. for 3 days. The total degradation impurities were measured by HPLC and summarized below in Table 25. The excipients were classified by the results as below.

TABLE 25

| Degradation total | |
|---|---|
| impurities (%) | Components (mass ratio of clofazimine and excipients) |
| 0-0.5 | PEG 3350 (1:400), |
| | RH 40 (1:100), |
| | Stearyl alcohol (1:70), |
| | liquid paraffin (1:1000), |
| | Vaseline (1:3000), |
| | propyl glycol (1:2500), |
| | glycerin (1:2500) |
| 0.5-1 | Oleic acid (1:50), |
| | labrasol (1:100), |
| | labrasol + water (1:100:800), |
| | labrasol + carbomer (1:100:5), |
| | transcutol + water (1:100:800) |
| >1 | Oleic acid + labrasol (1:50:100), |
| | oleic acid + labrasol + water (1:50:100:800), |
| | tween 80 (1:100), |
| | tween 80 + water (1:100:800), |
| | trascutol (1:100), |
| | transcutol + tween 80 (1:100:100), |
| | Campmul MCM (1:100), |
| | Campmul MCM + water (1:100:800), |
| | PEG 400 (1:400), |
| | Tefose 63 (1:100), |
| | Gelucire 44/14 (1:100) |

Clofazimine is easily hydrolyzed and oxidized. Excipients suitable to be used to prepare stable gels, ointments, or emulsions of clofazimine include labrasol, transcutol, propyl glycol, and glycerin. It was found that lipid excipients such as PEG 3350, stearyl alcohol, vaseline, and liquid paraffin could result in enhanced chemical stability of clofazimine.

### Example 16: Clofazimine Formulations

The topical formulations of Table 26 were prepared by the following. Clofazimine was dissolved in the mixture of stearyl alcohol, liquid paraffin and Vaseline at a temperature of 80° C., and then the resulting mixture was stirred and cooled to room temperature. The formulation base was prepared as the same method. No clofazimine crystal was observed by polarizing microscope in the three formulations, which means the clofazimine dissolved in the base.

TABLE 26

| Formulation 1 | Formulation 2 | Formulation 3 | Formulation base |
|---|---|---|---|
| Clofazimine | Clofazimine | Clofazimine | Stearyl alcohol |
| (0.01 g), | (0.1 g), | (0.5 g), | (4.3 g), |
| Stearyl alcohol | Stearyl alcohol | Stearyl alcohol | Liquid paraffin |
| (4.3 g), | (4.3 g), | (4.3 g), | (8.7 g), |
| Liquid paraffin | Liquid paraffin | Liquid paraffin | Vaseline (87 g) |
| (8.7 g), | (8.7 g), | (8.7 g), | |
| Vaseline (87 g) | Vaseline (86.9 g) | Vaseline (86.5 g) | |

### Example 17: Treatment of Clofazimine on Imiquimod-Induced Psoriasis Like Mouse Model

Fifty C57BL/6J mice were random divided into 5 groups, naïve group, model group, Formulation 1-3 groups. All mice were shaved on the back. Naïve group was the negative group and as the blank control. The other four groups were locally applied with 5% imiquimod cream (sold under the tradename Aldara®) once a day for a week. At the same time, each group was given Formulation base or one of Formulations 1-3 once a day for 7 consecutive days respectively. In the last three days, the changes of skin lesions were observed and evaluated by PASI method.

PASI method: the scores of erythema, scale and infiltration thickening in the skin lesions were scored respectively, and the sum of the three scores was the total score.

PASI scoring standard: 0, none; 1, mild; 2, moderate; 3, severe; 4, extremely severe.

The results were shown as below in Table 27 and confirmed the treatment effect of clofazimine on the imiquimod-induced psoriasis like mouse model.

TABLE 27

| Group | erythema | scale | thicken | scores |
|---|---|---|---|---|
| | Mean ± SEM | | | |
| | Day 5 | | | |
| Naive | 0 ± 0*** | 0 ± 0*** | 0 ± 0*** | 0 ± 0*** |
| Model | 1.3 ± 0.17 | 1.3 ± 0.21 | 1.4 ± 0.12 | 4 ± 0.25 |
| Formulation 1 | 0.4 ± 0.1*** | 0.4 ± 0.12** | 0.6 ± 0.12*** | 1.4 ± 0.26*** |
| Formulation 2 | 1.25 ± 0.19 | 0.45 ± 0.14** | 0.6 ± 0.12*** | 2.3 ± 0.25*** |
| Formulation 3 | 2.6 ± 0.39* | 0.85 ± 0.15 | 0.85 ± 0.13* | 4.3 ± 0.57 |
| | Mean ± SEM | | | |
| | Day 6 | | | |
| Naive | 0 ± 0*** | 0 ± 0*** | 0 ± 0*** | 0 ± 0*** |
| Model | 0.55 ± 0.09 | 0.95 ± 0.05 | 1.8 ± 0.13 | 3.3 ± 0.17 |
| Formulation 1 | 0.35 ± 0.11 | 0.45 ± 0.16* | 0.8 ± 0.2** | 1.6 ± 0.39** |
| Formulation 2 | 0.65 ± 0.11 | 0.6 ± 0.16 | 0.8 ± 0.08*** | 2.05 ± 0.25** |
| Formulation 3 | 1.1 ± 0.16** | 0.75 ± 0.11 | 1.1 ± 0.12** | 2.95 ± 0.24 |
| | Mean ± SEM | | | |
| | Day 7 | | | |
| Naive | 0 ± 0** | 0 ± 0*** | 0 ± 0*** | 0 ± 0*** |
| Model | 0.3 ± 0.08 | 0.45 ± 0.09 | 2.05 ± 0.19 | 2.8 ± 0.2 |
| Formulation 1 | 0.1 ± 0.07 | 0.25 ± 0.08 | 0.9 ± 0.1*** | 1.25 ± 0.2*** |
| Formulation 2 | 0.3 ± 0.08 | 0.25 ± 0.08 | 0.9 ± 0.07*** | 1.45 ± 0.17*** |
| Formulation 3 | 0.45 ± 0.12 | 0.35 ± 0.11 | 1.3 ± 0.11** | 2.1 ± 0.27* |

*p < 0.05,
**p < 0.01,
***p < 0.001 vs. Model group

### Example 18: Clofazimine Formulations

The topical formulations of Table 28 were prepared by the following. Clofazimine was dissolved in the mixture of stearyl alcohol, liquid paraffin and Vaseline at a temperature of 80° C., and then the resulting mixture was stirred and cooled to room temperature. Clofazimine crystal was observed by polarizing microscope in the two formulations (Formulations 4 and 5), which means clofazimine was not dissolved completely in the base.

TABLE 28

| Formulation 4 | Formulation 5 |
|---|---|
| Clofazimine (1 g), | Clofazimine (2 g), |
| Stearyl alcohol (4.3 g), | Stearyl alcohol (4.3 g), |
| Liquid paraffin (8.7 g), | Liquid paraffin (8.7 g), |
| Vaseline (87 g) | Vaseline (86.9 g) |

### Example 19: Stratum Corneum Penetration Effect of Different Topical Formulations

This experiment illustrates the stratum comeum penetration effect of different topical formulations on the nude mice skin.

Experiments were conducted in independent vertical Franz diffusion cells at 32° C.±0.5° C. at a rotation speed of 100 rpm. The acceptor compartment volume was 7 mL, with a diffusion area of 3.14 cm2. The acceptor compartment was filled with physiological saline solution. NU/NU nude mice skin was mounted, with the inner surface of the skin facing the saline diffusion solution (ensuring there were no air bubbles) and the stratum corneum exposed to the atmosphere. Clofazimine test preparation (0.5 g) was applied onto the skin and exposed for 4 hours, after which time the skin was removed from the device and cleaned by wiping with ethanol. The inner skin layer was isolated and the stratum corneum discarded. The inner skin later was cut into small pieces, transferred to a 100 mL volumetric flask and extracted by immersion in acetonitrile for 24 hours. The clofazimine content in the acetonitrile extract was determined by HPLC, and the clofazimine content per unit area of epidermis calculated.

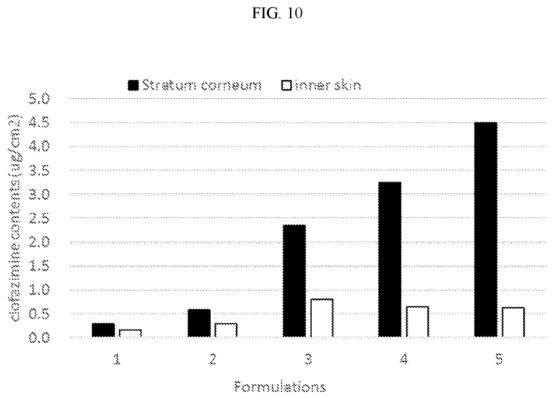

As illustrated in FIG. 10, the results showed that increasing clofazimine contents in the base would increase its contents in the stratum comeum; however, it would reach a peak by Formulation 3 (clofazimine dissolved in the base completely) and would not increase anymore by Formulation 4 and 5 (clofazimine dissolved in the base incompletely). The results indicated that only dissolved clofazimine can penetrate the stratum comeum.

### Example 20: Clofazimine Formulations

The topical formulations of Table 29 were prepared as the following. Clofazimine was dissolved in a mixture of liquid paraffin and Vaseline at temperature 80° C., and then the resultant mixture was stirred and cooled to the room temperature. No API crystal was observed by polarizing microscope in the three formulations, which means the API dissolved in the base.

TABLE 29
Topical Formulations

| Formulation 6 | Formulation 7 | Formulation 8 | Formulation 9 |
|---|---|---|---|
| Clofazimine | Clofazimine | Clofazimine | Clofazimine |
| (0.5 g) | (0.05 g) | (0.5 g) | (0.05 g) |
| Liquid paraffin | Liquid paraffin | Vaseline | Vaseline (500 g) |
| (100 g) | (100 g) | (500 g) | |
| Vaseline (400 g) | Vaseline (400 g) | | |

The in vivo PK profile of Formulations 6-9 will be investigated by applying the clofazimine topical formulations on minipig skin.

### Example 21: Inhibition of Kv1.3 Channels in Human Jurkat E6-1 Cells

Voltage-gated potassium channels of the Kv1.3 type are widely expressed in many types of cells, both normal and cancer. Activity of Kv1.3 channels plays an important role in setting the resting membrane potential, cell proliferation, apoptosis and volume regulation. Altered expression of Kv1.3 channels have been found in some cancer diseases such as breast, colon, pancreas, smooth muscle, skeletal muscle, lung, kidney and prostate cancer. Inhibitors of Kv1.3 channels evidence clinical application in therapy of some cancer disorders characterized by an over-expression of Kv1.3 channels, such as, for example, breast and lung cancer, melanoma, or chronic lymphocytic leukaemia.

The influence of clofazimine on the activity of the channels was studied by applying the voltage ramp protocol to a human Jurkat E6-1 cell line. The cell line was cultured in RPMI 1640 medium with 10% FBS and passaged. Voltage ramps depolarizing cell membranes from −100 mV up to 100 mV were applied every 2 s; the ramp duration was 50 ms and holding potential −70 mV. Upon application of the voltage ramp protocol, after Kv1.3 currents in Jurkat E6-1 cells were stable, the test formulations were added and detected. Formulations of clofazimine at 0.3 μM, 1 μM, 3 μM, 10 μM, and 20 μM in DMSO are shown below in Table 30. Clofazimine was found to inhibit Kv1.3 current in Jurkat E6-1 cells, and the fitted IC50 value was 3.3 μM±657.0 nM.

TABLE 30

| Inhibition ratio of Kv1.3 channel current in Jurkat E6-1 cells (%) | | | | | | |
|---|---|---|---|---|---|---|
| 0.3 μM | 1 μM | 3 μM | 10 μM | 20 μM | n | IC₅₀ |
| 7.09 ± 0.86% | 18.89 ± 3.38% | 46.84 ± 4.55% | 70.11 ± 2.91% | 83.62 ± 1.99% | 3 | 3.3 ± 0.657 μM |

### Example 22: Inhibition of IL-2 Secretion of Activated Jurkat Cells

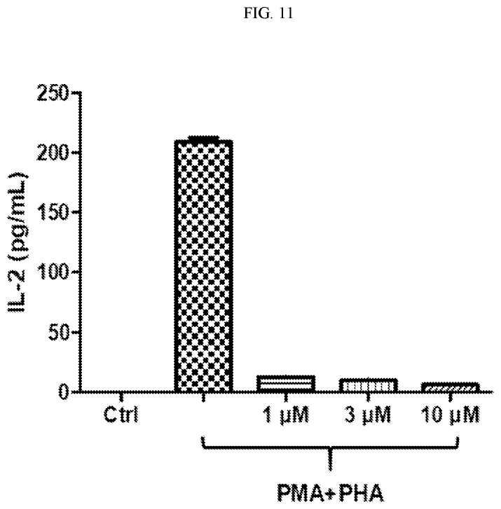

Jurkat cells were divided into 5 groups: negative (no treatment), positive control (PMA+PHA), and formulations of clofazimine at 1 μM, 3 μM, and 10 μM in DMSO. In each group, Jurkat cells were incubated with corresponding formulation. After 24 h, the secretion of IL-2 was detected by ELISA. The secretion of IL-2 in positive control group increased significantly; and was significantly inhibited by the clofazimine formulations (FIG. 11).

### Example 23 Clofazimine Formulations to Treat Psoriasis in Mice

| Formulation | 10 | 11 | 12 |
|---|---|---|---|
| Clofazimine (wt %) | 0.01% | 0.03% | 0.1% |
| White Vaseline (wt %) | 79.99% | 79.97% | 79.9% |
| Light liquid paraffin (wt %) | 20% | 20% | 20% |

Studies were performed comparing the effects of a once daily topical treatment of benvitimod cream (1%) (Zhonghao Pharma, China), calcipotriol ointment (15 g:75 mg) (Daivonex, LEO Pharma A/S, Denmark), Formulation 10 (clofazimine 0.01%), Formulation 11 (clofazimine 0.03%) and Formulation 12 (clofazimine 0.1%) in imiquimod induced psoriasis-like mice. The psoriasis-like skin inflammation mouse model was generated by daily topical application of imiquimod cream (5%) (Aldara, INOVA Pharmaceuticals, Singapore) on the back of mice for 9 consecutive days.

The benvitimod cream (1%) was found to significantly reduce skin lesions. The calcipotriol ointment (15 g:75 mg) ameliorated skin erythema, but caused skin damage, peeling and thickening due to damage on skin, resulting in no improvement in terms of scales and skin thickening. Formulations 10-12 (clofazimine 0.01%, 0.03% and 0.1%) significantly reduced skin lesions in a concentration-dependent manner. The efficacy of Formulation 12 (0.1%) was superior to that of Benvitimod cream (0.1%) with respect to reducing skin inflammation.

Benvitimod cream (1%), calcipotriol ointment (15 g:75 mg) and Formulations 10-12 (clofazimine 0.01%, 0.03% and 0.1%) each reduced micro abscesses in the cuticle, decreased the thickening of cuticle, and reduced inflammatory infiltration into the dermis. The benvitimod cream was slightly superior to that of calcipotriol cream in these respects. The effects of Formulations 10 and 11 (clofazimine 0.01% and 0.03%) were comparable to that of the calcipotriol ointment, and the effects of Formulation 12 (0.1%) was comparable to the benvitimod cream.

### Example 24 Clofazimine Blood Concentrations in Minipigs

Studies were performed to evaluate the blood concentration-time curve after topical application of Formulations 10-12 (clofazimine 0.01%, 0.03% and 0.1%) in Bama minipigs. 24 Bama minipigs (12 male and 12 female) were randomly divided into 4 groups (n=6), three groups received a single topical application of Formulations 10-12 (clofazimine 0.01%, 0.03% and 0.1%). A fourth group received repeated topical application of Formulation 11 (clofazimine 0.03%, bid, 12 h interval, 9 applications in total). In above 4 groups, Formulations 10-12 were applicated to 5% body surface area of Bama minipigs, that is, 1.5 mg ointment/cm² (corresponding to 0.15, 0.45, 1.5 μg API/cm² respectively). For the single dose groups, blood samples were collected at baseline, 0.167, 0.5, 1, 2, 4, 6, 8, 12, 24, 48 and 72 hours after drug application. Blood samples in the repeat dose group were collected before 5^{th} to 8^{th} topical application, 0.167, 0.5, 1, 2, 4, 6, 8, 12, 24, 48 and 72 hours after drug application. A validated LC-MS/MS method was used to determine the concentration of clofazimine in plasma and the pharmacokinetic parameters were calculated (the standard curve range of clofazimine is 0.0100˜10.0 ng/mL).

For single dose groups, a minority of samples in the 0.01% and 0.1% groups showed a very low level of clofazimine (close to the detection limit of the instrument). In the majority of the samples no clofazimine was detectable. For repeated dose groups, a very low level of clofazimine could be detected in plasma. No significant gender difference in clofazimine absorption was observed.

### Example 25 Clofazimine Tissue Distribution in Minipigs

Studies were performed to evaluate the pharmacokinetics and tissue distribution after a single-dose topical application of Formulation 11 (clofazimine 0.03%) in Bama minipigs. 24 Bama minipigs were divided into 6 groups (n=4), each group of minipigs received a single topical application of Formulation 11 (0.03%) at a dose of 1.5 mg ointment/cm². Blood samples, epidermis, dermis, subcutaneous tissue and subcutaneous muscle were collected at 0.5, 4, 24, 48, 72 and 96 hours (one time point for one group) after drug application. A validated LC-MS/MS method was used to determine the concentration of clofazimine in plasma, epidermis, dermis, subcutaneous tissues and subcutaneous muscle and the pharmacokinetic parameters were calculated (the standard curve range of clofazimine is 0.0100˜10.0 ng/mL).

At each time point, the distribution trend of clofazimine was as follows: epidermis >dermis >subcutaneous tissue subcutaneous muscle >plasma. The median time to peak epidermal and subcutaneous muscle concentrations was about 0.5 h, while the median time to peak dermal and subcutaneous tissues concentrations was about 4 h. The concentration of clofazimine in epidermis and dermis was much higher than that in subcutaneous tissue and muscle, indicating that the clofazimine was mainly distributed in the epidermis and dermis, and not absorbed into blood. The results of the studies are shown below in Table 31.

TABLE 31

| Sample | 0.5 h | 4 h | 24 h | 48 h | 72 h | 96 h |
|---|---|---|---|---|---|---|
| Epidermis | 829 ± 238 | 669 ± 668 | 161 ± 134 | 249 ± 197 | 681 ± 707 | 553 ± 893 |
| Dermis | 19.9 ± 10.6 | 62.7 ± 51.0 | 58.3 ± 51.6 | 46.9 ± 39.4 | 45.0 ± 19.4 | 22.0 ± 13.2 |
| subcutaneous | 0.178 | 0.648 | 0.139 | 0.122 | 0.232 | 0.221 |
| tissues | | | | | | |
| subcutaneous | 0.744 ± 1.05 | 0.267 ± 0.166 | 0.531 ± 0.325 | 0.115 ± 0.0407 | 0.0923 | 0.176 ± 0.114 |
| muscle | | | | | | |
| Plasma | 0.0135 | / | / | / | 0.0124 | / |

### Example 26 Dermal Irritation in Rabbits

Studies were performed to evaluate the dermal irritation after a 28 day topical treatment of Japanese White Rabbits with Formulation 11 and 12. 24 rabbits (12 males and 12 females) were divided into 6 groups (n=4), including 3 intact skin groups and 3 damaged skin groups. Plain ointment, Formulation 11 (0.03%) and Formulation 12 (0.1%), at a dose of 0.4 g/rabbit, were applied twice daily on the right shaved area in both damaged skin and intact skin. A similar amount of normal saline was applied on the left side as a control for consecutive 28 days. The general condition of all groups was good, and no skin irritation was observed at the administration site.

### Example 27 Skin Phototoxicity in Guinea Pig Back Skin

Studies were performed to evaluate the skin phototoxicity of Formulation 11 and 12 in guinea pigs. 40 Hartley guinea pigs (20 males and 20 females) were divided into 5 groups (n=8). Two squares of 5×5 cm/site on the back skin were shaved. Normal saline (negative control, 0.18 mL/side), 0.1% 8-MOP (positive control, 0.2 mL/side), plain ointment (0.18 g/side), Formulation 11 (0.03%, 0.18 g/side) and Formulation 12 (0.1%, 0.18 g/side) were applied to shaved skin. After 30 min the drugs were washed off, and the right side was UV-irradiated at 10 J/cm² for 18 min and the left side was covered with a sheet of aluminum foil (nonirradiated part). The skin reactions at 1, 24, 48, and 72 h after irritation were evaluated. No toxic reaction or phototoxicity was observed.

### Example 26 Skin Phototoxicity in Guinea Pig Nuchal Skin

Studies were performed to evaluate the skin phototoxicity of Formulation 11 and 12 in guinea pigs. 50 Hartley guinea pigs (25 males and 25 females) were divided into 5 groups (n=10)). The nuchal skin (4×4 cm) of each subject was shaved. Normal saline (negative control, 0.18 mL), 1% TCSA (positive control, 0.2 mL), plain ointment (0.18 g), Formulation 11 (0.03%, 0.18 g) and Formulation 12 (0.1%, 0.18 g) were applied to shaved skin for 5 days (1 time/d). Following last sensitization, the guinea pigs were challenged for 2 weeks. NS (0.18 mL/side), 0.5% TCSA (0.03 mL/side), plain ointment (0.18 g/side), Formulation 11 (0.03%, 0.18 g/side) and Formulation 12 (0.1%, 0.18 g/side) were applied to each side of shaved back skin. After 30 min the drugs were washed off, and the right side was UV-irradiated at 10 J/cm² for 19 min and the left side was covered with a sheet of aluminum foil. The skin reactions at 1, 24, 48, and 72 h after challenge were evaluated. No photo hypersensitivity reaction was observed.

### Example 27 Anaphylaxis in Guinea Pigs

Studies were performed to evaluate the active Cutaneous Anaphylaxis of Formulation 11 and 12 in guinea pigs. 50 Hartley guinea pigs (25 males and 25 females) were divided into 5 groups (n=10). The left side of the back skin for each guinea pig was shaved. Normal saline (negative control, 0.18 mL), 1% DNCB (positive control, 0.2 mL), plain ointment (0.18 g), Formulation 11 (0.03%, 0.18 g) and Formulation 12 (0.1%, 0.18 g) were applied to shaved skin at day 1, 8 and 15. 14 days after last sensitization, the guinea pigs were challenged. NS (0.18 mL), 0.5% DNCB (0.2 mL), plain ointment (0.18 g), 0.03% Formulation 11 (0.18 g) and 0.1% Formulation 12 (0.18 g) were applied to the right side of shaved back skin. The administration sites of each group were covered with gauze and cellophane for sealing and fixation for about 6 hours, then the gauze and cellophane were removed and skin reactions at 1, 24, 48, and 72 h thereafter were evaluated. The results of active cutaneous anaphylaxis were negative.

### Example 28 Stability of Formulations 10-12

Accelerated and long term stability experiments were performed on Formulation 10, Formulation 11, and Formulation 12. The assay procedure follows USP 40 clofazimine capsule method and its details are shown below in Table 32.

TABLE 32
Assay procedure for measuring stability

| | |
|---|---|
| Buffer | 4.5 mg/mL of sodium dodecyl sulfate, 1.7 mg/mL |
| | of tetrabutylammonium hydrogen sulfate, and 1.8 |
| | mg/mL of disodium hydrogen phosphate in water. |
| | Adjust with dilute phosphoric acid (about 8.5%) |
| | to a pH of 3.0 in 90% of the volume before |
| | diluting with water to volume. |
| Mobile phase | Acetonitrile and Buffer (65:35) |
| Chromatographic | See Chromatoagraphy <621>, System Suitability. |
| system | |
| Mode | LC |
| Detector | UV 280 nm. For Identification test B, use a diode |
| | array detector in the range of 190 nm-400 nm. |
| Column | 4.6-mm × 25-cm; 5-μm packing L7 |
| Flow rate | 1.0 mL/min |
| Injection volume | 20 μL |

The impurities were measured by HPLC and the parameters and procedures of HPLC are provided below and in Table 33. Phenylsilane bonded silica gel was used as filler (Agilent, ZORBAX SB Phenyl 250 mm 4.6 mm, 5 μm or equivalent column). The mobile phase A, acetonitrile and methanol were used as the mobile phase C with pH 3.0 buffer salt solution (4.5 g sodium dodecyl sulfate, 1.7 g tetrabutylammonium bisulphate and 1.8 g disodium hydrogen phosphate dodecahydrate dissolved in 900 mL water, adjusted the pH value to 3.0 with 8.5% phosphoric acid, and then diluted to 1 L with water) The flow rate was 1.0 mL/min. The detection wavelength was 280 nm. Gradient elution was performed at column temperature 30 as shown in the Table 33 below.

TABLE 33
HPLC parameters

| Time (min) | A (%) | B (%) | C (%) |
|---|---|---|---|
| 0 | 26 | 20 | 54 |
| 28 | 26 | 20 | 54 |
| 50 | 20 | 80 | 0 |
| 60 | 20 | 80 | 0 |
| 65 | 26 | 20 | 54 |
| 75 | 26 | 20 | 54 |

The results of the experiments are shown below in Tables 34-39. The accelerated stability experiments were conducted at 40° C. and 75% RH. The long term stability experiments were conducted at 25° C. and 60% RH. The abbreviation “NT” denotes that the sample was not tested at the time point.

TABLE 34
Accelerated stability of Formulation 10

| Test | | Specification | 0 M | 1 M | 2 M | 3 M | 6 M |
|---|---|---|---|---|---|---|---|
| Character | | The appearance of ointment | Conforms | Conforms | Conforms | Conforms | Conforms |
| | | should be orange yellow | | | | | |
| Identification | | The retention time of the | Conforms | Conforms | Conforms | Conforms | Conforms |
| | | main peak of the solution | | | | | |
| | | should be consistent with | | | | | |
| | | that of the reference solution | | | | | |
| Uniformity | | The ointment should be fine, | Conforms | Conforms | Conforms | Conforms | Conforms |
| | | evenly distributed and free | | | | | |
| | | from agglomeration | | | | | |
| Related | Single | Not more than 1.0% | 0.10% | 0.10% | 0.10% | 0.10% | 0.11% |
| substances | impurity | | (RRT0.81) | (RRT0.81) | (RRT0.81) | (RRT0.81) | (RRT0.80) |
| | Total | Not more than 2.0% | 0.10% | 0.10% | 0.10% | 0.10% | 0.11 |

| | | | | | | |
|---|---|---|---|---|---|---|
| Assay (%) | Based on the initial | 99.6 | 99.9 | 100.0 | 100.4 | 99.4 |
| | clofazimine concentration | | | | | |
| Microbial | Aerobe: ≤10² CFU/g; mold | Conforms | NT | NT | NT | NT |
| limits | and yeast: ≤10¹ CFU/g; | | | | | |
| | Staphylococcus aureus and | | | | | |
| | Pseudomonas aeruginosa | | | | | |
| | should not be detected. | | | | | |

TABLE 35
Accelerated stability of Formulation 11

| Test | | Specification | 0 M | 1 M | 2 M | 3 M | 6 M |
|---|---|---|---|---|---|---|---|
| Character | | The appearance of ointment | Conforms | Conforms | Conforms | Conforms | Conforms |
| | | should be orange yellow | | | | | |
| Identification | | The retention time of the | Conforms | Conforms | Conforms | Conforms | Conforms |
| | | main peak of the solution | | | | | |
| | | should be consistent with | | | | | |
| | | that of the reference solution | | | | | |
| Uniformity | | The ointment should be fine, | Conforms | Conforms | Conforms | Conforms | Conforms |
| | | evenly distributed and free | | | | | |
| | | from agglomeration | | | | | |
| Related | Single | Not more than 1.0% | 0.10% | 0.10% | 0.10% | <0.10% | 0.11% |
| substances | impurity | | (RRT0.81) | (RRT0.81) | (RRT0.81) | (0.09%) | (RRT0.80) |
| | | | | | | (RRT0.81) | |
| | Total | Not more than 2.0% | 0.10% | 0.10% | 0.10% | <0.10% | 0.11% |
| | | | | | | (0.09%) | |

| | | | | | | |
|---|---|---|---|---|---|---|
| Assay (%) | Based on the initial | 100.8 | 100.0 | 99.8 | 99.5 | 98.8 |
| | clofazimine concentration | | | | | |
| Microbial | Aerobe: ≤10² CFU/g; mold | Conforms | NT | NT | NT | NT |
| limits | and yeast: ≤10¹ CFU/g; | | | | | |
| | Staphylococcus aureus and | | | | | |
| | Pseudomonas aeruginosa | | | | | |
| | should not be detected. | | | | | |

TABLE 36
Accelerated stability of Formulation 12

| Test | | Specification | 0 M | 1 M | 2 M | 3 M | 6 M |
|---|---|---|---|---|---|---|---|
| Character | | The appearance of ointment | Conforms | Conforms | Conforms | Conforms | Conforms |
| | | should be orange red | | | | | |
| Identification | | The retention time of the | Conforms | Conforms | Conforms | Conforms | Conforms |
| | | main peak of the solution | | | | | |
| | | should be consistent with | | | | | |
| | | that of the reference solution | | | | | |
| Uniformity | | The ointment should be fine, | Conforms | Conforms | Conforms | Conforms | Conforms |
| | | evenly distributed and free | | | | | |
| | | from agglomeration | | | | | |
| Related | Single | Not more than 1.0% | 0.10% | 0.10% | 0.11% | 0.10% | 0.11% |
| substances | impurity | | (RRT0.81) | (RRT0.81) | (RRT0.81) | (RRT0.81) | (RRT0.80) |
| | Total | Not more than 2.0% | 0.10% | 0.10% | 0.11% | 0.10% | 0.11% |

| | | | | | | |
|---|---|---|---|---|---|---|
| Assay (%) | Based on the initial | 100.3 | 100.1 | 99.1 | 99.8 | 99.0 |
| | clofazimine concentration | | | | | |
| Microbial | Aerobe: ≤10² CFU/g; mold | Conforms | NT | NT | NT | NT |
| limits | and yeast: ≤10¹ CFU/g; | | | | | |
| | Staphylococcus aureus and | | | | | |
| | Pseudomonas aeruginosa | | | | | |
| | should not be detected. | | | | | |

TABLE 37
Long term stability of Formulation 10

| Test | | Specification | 0 M | 3 M | 6 M | 9 M |
|---|---|---|---|---|---|---|
| Character | | The appearance of ointment | Conforms | Conforms | Conforms | Conforms |
| | | should be orange yellow | | | | |
| Identification | | The retention time of the | Conforms | Conforms | Conforms | Conforms |
| | | main peak of the solution | | | | |
| | | should be consistent with | | | | |
| | | that of the reference solution | | | | |
| Uniformity | | The ointment should be fine, | Conforms | Conforms | Conforms | Conforms |
| | | evenly distributed and free | | | | |
| | | from agglomeration | | | | |
| Related | Single | Not more than 1.0% | 0.11% | 0.10% | 0.11% | 0.13% |
| substances | impurity | | (RRT0.81) | (RRT0.81) | (RRT0.80) | (RRT0.80) |
| | Total | Not more than 2.0% | 0.11% | 0.10% | 0.11% | 0.13% |

| | | | | | |
|---|---|---|---|---|---|
| Assay (%) | Based on the initial | 99.6 | 100.1 | 99.9 | 101.5 |
| | clofazimine concentration | | | | |
| Microbial | Aerobe: ≤10² CFU/g; mold | Conforms | NT | NT | NT |
| limits | and yeast: ≤10¹ CFU/g; | | | | |
| | Staphylococcus aureus and | | | | |
| | Pseudomonas aeruginosa | | | | |
| | should not be detected. | | | | |

TABLE 38
Long term stability of Formulation 11

| Test | | Specification | 0 M | 3 M | 6 M | 9 M |
|---|---|---|---|---|---|---|
| Character | | The appearance of ointment | Conforms | Conforms | Conforms | Conforms |
| | | should be orange yellow | | | | |
| Identification | | The retention time of the | Conforms | Conforms | Conforms | Conforms |
| | | main peak of the solution | | | | |
| | | should be consistent with | | | | |
| | | that of the reference solution | | | | |
| Uniformity | | The ointment should be fine, | Conforms | Conforms | Conforms | Conforms |
| | | evenly distributed and free | | | | |
| | | from agglomeration | | | | |
| Related | Single | Not more than 1.0% | 0.10% | 0.10% | 0.11% | 0.12% |
| substances | impurity | | (RRT0.81) | (RRT0.81) | (RRT0.80) | (RRT0.81) |
| | Total | Not more than 2.0% | 0.11% | 0.10% | 0.11% | 0.12% |

| | | | | | |
|---|---|---|---|---|---|
| Assay (%) | Based on the initial | 100.8 | 99.0 | 98.3 | 100.1 |
| | clofazimine concentration | | | | |
| Microbial | Aerobe: ≤10² CFU/g; mold | NT | NT | NT | NT |
| limits | and yeast: ≤10¹ CFU/g; | | | | |
| | Staphylococcus aureus and | | | | |
| | Pseudomonas aeruginosa | | | | |
| | should not be detected. | | | | |

TABLE 39
Long term stability of Formulation 12

| Test | | Specification | 0 M | 3 M | 6 M | 9 M |
|---|---|---|---|---|---|---|
| Character | | The appearance of ointment | Conforms | Conforms | Conforms | Conforms |
| | | should be orange red | | | | |
| Identification | | The retention time of the | Conforms | Conforms | Conforms | Conforms |
| | | main peak of the solution | | | | |
| | | should be consistent with | | | | |
| | | that of the reference solution | | | | |
| Uniformity | | The ointment should be fine, | Conforms | Conforms | Conforms | Conforms |
| | | evenly distributed and free | | | | |
| | | from agglomeration | | | | |
| Related | Single | Not more than 1.0% | 0.10% | 0.10% | 0.11% | 0.12% |
| substances | impurity | | (RRT0.81) | (RRT0.81) | (RRT0.80) | (RRT0.81) |
| | Total | Not more than 2.0% | 0.10% | 0.10% | 0.11% | 0.12% |

| | | | | | |
|---|---|---|---|---|---|
| Assay (%) | Based on the initial | 100.3 | 100.2 | 99.0 | 100.9 |
| | clofazimine concentration | | | | |
| Microbial | Aerobe: ≤10² CFU/g; | NT | NT | NT | NT |
| limits | mold and yeast: ≤10¹ CFU/g; | | | | |
| | Staphylococcus aureus and | | | | |
| | Pseudomonas aeruginosa | | | | |
| | should not be detected. | | | | |

### Example 29. Administration of the Herein Described Topical Clofazimine Formulations to Patients

This study is a single-center, double-blind, randomized, placebo-controlled clinical trial of the efficacy and safety of clofazimine ointments in the treatment of plaque psoriasis. The study will enroll about 32 subjects. The subjects will be randomly assigned to 4 groups of 8 in a 1:1:1:1 ratio.

The inclusion criteria for this study can include one or more of the following:

1. Willing to participate in the clinical study, fully understanding and informed of the study and signing the informed consent, willing to follow and able to complete all trial procedures;
2. No gender limitation, age 18˜65 at the time of signing the informed consent;
3. Female patients should not be pregnant and not plan to become pregnant during the trial, and effective contraceptive measures should be taken during the trial;
4. Male patients should agree to abstain from sex or use contraception during the trial;
5. Clinically diagnosed with plaque psoriasis for at least 6 months;
6. Total skin lesion area: 2-10% body surface area;
7. Investigator's overall evaluation (PGA) 2;
8. Observe the skin lesion diameter of the target site 3 cm and comply with external medication treatment;
9. Previous diseases such as heart disease, liver, kidney, digestive tract, nervous system, mental disorder and metabolic disorder.

The exclusion criteria for this study can include one or more of the following:

1. Exclusion based on target diseases
(a). Currently diagnosed with unusual psoriasis (e.g. erythroderma, pustular, and arthropathic psoriasis), and advanced psoriasis vulgaris;
(b). Psoriasis caused by drugs (e.g., lithium agents, beta blockers, antimalarials, angiotensin conversion inhibitors (ACEI), etc.);
(c). Psoriasis lesions greater than 10% of the body surface area;
(D). Palm-plantar psoriasis;
(e). Patients with other skin diseases that may interfere with the assessment of their condition;
(f). Patients with severe skin breakage and infection at the lesion site;
2. Exclusion based on medical history and accompanying diseases
(a) with severe organs and systemic diseases, including cardiovascular (e.g., heart failure, unstable angina pectoris), liver (such as cirrhosis of the liver), kidney (such as renal failure), lung (such as chronic obstructive pulmonary disease), endocrine (such as Cushing's syndrome, diabetes, obesity (BMI >25)), osteoarthritis, the digestive tract, nervous system and blood system diseases, etc., and other autoimmune diseases, malignant tumors, or researchers think doesn't fit to participate in this study to other diseases;
(b). Any of the following abnormalities are found in the laboratory tests during screening: peripheral white blood cell count <3.0×109/L or >15×109/L; Hemoglobin <90 g/L; Platelet <100×109/L; The upper limit of ALT and AST>normal value is 1.5 times; Serum creatinine and BUN are higher than the upper limit of normal.
(c). Conditions that may affect patient compliance, such as long-term business or leave, mental illness;
3. Exclusion based on previous and current treatments
(a). Received topical psoriatic drugs (such as glucocorticoids, vitamin D3 derivatives, retinoates, dianthracene, coal tar, salicylic acid, calcineurin inhibitors and local immune modulators) within 2 weeks before screening;
(b). Phototherapy (UVA, UVB) had been received within 4 weeks before screening;
(c). Received systemic drugs (retinoids, MTX, cyclosporine, glucocorticoids, Chinese patent medicine, etc.) within 4 weeks before screening;
(d). Received systemic biologics known to affect psoriasis (whether marketed or not) within a specified period prior to screening: ustekinumab, 32 weeks before initial treatment; Secukinumab, 22 weeks before initial administration; Efalizumab, 18 weeks before initial administration; Alefacept, infliximab, adalimumab, 8 weeks before the first treatment; Etanercept, 4 weeks before initial administration; For other drugs, 4 weeks/5 half-life before first administration (whichever is the longer);
(e). Participating in other clinical studies, or planning to begin treatment in this study less than 3 months after the end of drug treatment in the previous clinical study;
(f). Regular use of herbal medicines or sedatives, sleeping pills, tranquilizers and other addictive drugs;
4. Participants have a history of allergy to the active ingredient or excipient of the study drug;
5. Pregnant women, lactating women, women with positive pregnancy tests at the time of inclusion or before administration of the study drug;
6. According to the researcher's judgment, participants have other conditions that are not suitable for inclusion.

Topical clofazimine formulations will be administered at 3 doses (e.g., Formulations 10, 11 and 12). Placebo (blank matrix) will be administered as well.

Intervention: For external skin use, wash the affected area, after the skin is dry, apply proper amount of this product evenly on the affected area, avoid contact with normal skin, once a day in the morning and evening. At least 1 hour after each application, can be wiped or cleaned, the application of skin lesions, including the treatment of new psoriasis skin lesions. For all original affected areas, administration should continue until the end of the study, even after the lesions have disappeared. For the purpose of this study, this drug will not be applicable to face, armpit, vulva and other parts.

Study process: This study is divided into three stages:

Screening period;
Treatment period (1, 4, 8 weeks, until intolerable toxicity occurs, the participant or physician decides to discontinue treatment, or for other reasons specified by the protocol, whichever occurs first);
Return visit period after drug withdrawal (2, 4 weeks)

Outcome Measures: The primary outcome measures will be PASI75 and PGA, the secondary outcome measures will be PASI50 and PASI90, and the safety outcome measures will include ADR and its incidence.

Data statistics are performed by biological statisticians using statistical software. All hypothesis tests will be two-sided, and P<0.05 will be considered statistically significant.

Process diagram. An exemplary process diagram is illustrate below and in scheme 1:

| | | | Visit during treatment | | | Visit after drug withdrawal | |
|---|---|---|---|---|---|---|---|
| Visit | Screen | Baseline | 1 week | 4 week | 8 week | 2 week | 4 week |
| Informed consent | X | | | | | | |
| Demographic data | X | | | | | | |
| Medical history | X | | | | | | |
| Physical examination | X | | | | | | |
| Comorbidity | X | | | | | | |
| Concomitant medication | X | | | | | | |
| Urine pregnancy test | X | | | | X | | |
| Blood test | X | | | X | X | | |
| Blood biochemical test | X | | | X | X | | |
| HBsAg, HCVAb, HIV | X | | | | | | |
| and RPR tests | | | | | | | |
| Inclusion/exclusion criteria | X | | | | | | |
| Skin lesion | | X | X | X | X | X | X |
| assessment/photography | | | | | | | |
| Skin lesion area measurement | | X | X | X | X | X | X |
| Quality of life assessment | | X | X | X | X | X | X |
| Distribution of test drug | | X | X | X | X | | |
| Return of test drug | | | | | X | | |
| Subject diary card issuance | | X | | | | | |
| Return of the | | | | | X | | |
| subject diary card | | | | | | | |
| Compliance assessment | | | X | X | X | | |
| Adverse events | | | X | X | X | X | X |

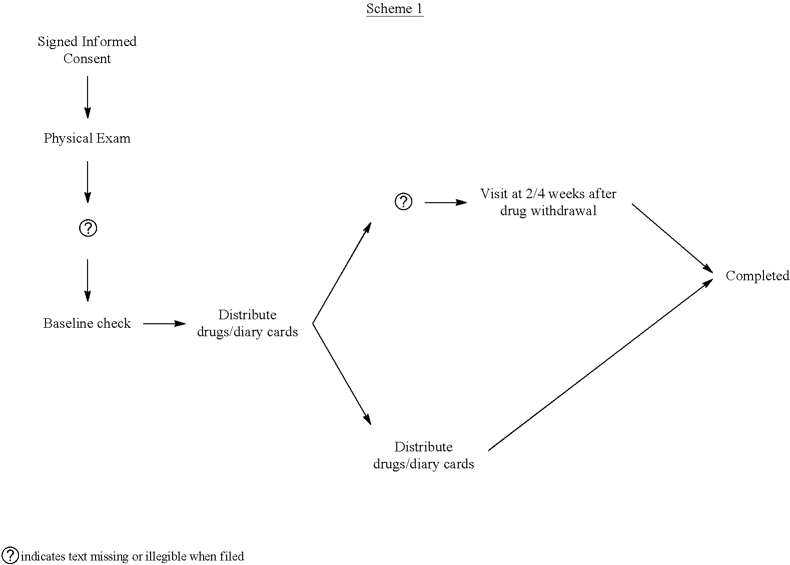

For the purpose of this study, all participants will sign an informed consent form before conducting research-related inspections, women of childbearing age will undergo a urine pregnancy test with negative results and be provided with effective contraception during the study period. Routine blood tests can be included, e.g., hemoglobin, red blood cell count, white blood cell count, and platelet count. Blood biochemical tests can be included, e.g., alanine aminotransferase, aspartate aminotransferase, urea nitrogen, creatinine, triglyceride, and total cholesterol.

Exemplary overall visit arrangement: Participants' visits can occur at baseline, at 1, 4, and 8 weeks of medication, and at 2, 4 weeks after drug withdrawal. During the two visits, if the disease progresses, the participants can contact the investigator at any time, and visit the hospital in time. After the investigator judges that the patient has relapsed, he/she will take the drug again.

### Example 30. Formulations of APIs in Table 1

Formulations are made by replacing clofazimine in Formulations 1-12 with the APIs in Table 1 (other than clofazimine). The preparation procedure is the same as the preparation procedure for Formulations 1-12 as provided in their respective examples.

1. A topical pharmaceutical composition that comprises:
(a) clofazimine or a pharmaceutically acceptable salt thereof, present in an amount of up to about 1.5% of the total weight of the composition,
and
(b) at least one solubilizer,
wherein the pharmaceutical composition is formulated for topical administration.

2. The pharmaceutical composition of claim 1, wherein the compound or the pharmaceutically acceptable salt thereof is dissolved in the at least one solubilizer.

3. (canceled)

4. The pharmaceutical composition of claim 1, wherein the pharmaceutical composition is stable for 3 months at 25° C. and 60% RH.

5. The pharmaceutical composition of claim 1, wherein a total impurity amount in the pharmaceutical composition is no more than 2% w/w based on the initial amount of the compound or a pharmaceutically acceptable salt thereof after stored for 6 months at 40° C. and 75% RH.

6-13. (canceled)

14. The pharmaceutical composition of claim 1, wherein the compound or the pharmaceutically acceptable salt thereof is present in an amount of up to about 1% of the total weight of the pharmaceutical composition.

15-25. (canceled)

26. The pharmaceutical composition of claim 1, wherein the pharmaceutical composition is non-aqueous.

27. (canceled)

28. The pharmaceutical composition of claim 1, wherein the at least one solubilizer comprises one or more of the following: (i) petroleum jelly, (ii) one or more alkanes each independently having at least nine carbons, (iii) a fatty alcohol having at least nine carbons; and (iv) a fatty acid having at least nine carbons.

29. (canceled)

30. (canceled)

31. The pharmaceutical composition of claim 28, wherein the at least one solubilizer is present in an amount of from about 50% to about 99.9% of the total weight of the pharmaceutical composition.

32. (canceled)

33. The pharmaceutical composition of claim 28, wherein the at least one solubilizer comprises petroleum jelly.

34. The pharmaceutical composition of claim 33, wherein the petroleum jelly is present in an amount of from about 20% to about 99.9% of the total weight of the pharmaceutical composition.

35-37. (canceled)

38. The pharmaceutical composition of claim 28, wherein the at least one solubilizer comprises one or more alkanes each independently having at least nine carbons.

39. (canceled)

40. (canceled)

41. The pharmaceutical composition of claim 38, wherein the one or more alkanes are liquid paraffin.

42. The pharmaceutical composition of claim 41, wherein the one or more alkanes are present in an amount of from about 0.5% to about 30% of the total weight of the pharmaceutical composition.

43-60. (canceled)

61. The pharmaceutical composition of claim 1, consisting of:
(a) clofazimine in an amount of about 0.01% to about 0.1% of the total weight of the pharmaceutical composition;
(b) vaseline in an amount of about 75% to about 85% of the total weight of the pharmaceutical composition;
(c) liquid paraffin in an amount of about 15% to about 25% of the total weight of the pharmaceutical composition.

62-110. (canceled)

111. A method of treating a skin disease, comprising administering the pharmaceutical composition of claim 1 to a subject in need thereof.

112. The method of claim 111, wherein the skin disease is caused by immune inflammation.

113. The method of claim 112, wherein the skin disease is psoriasis, vitiligo, lupus erythematosus, chronic eczema, atopic dermatitis, dermatitis or contact dermatitis.

114. (canceled)

115. The method of claim 112, wherein the skin disease is plaque psoriasis.

116. (canceled)

117. The pharmaceutical composition of claim 28, wherein the at least one solubilizer further comprises (i) esters with propylene glycol or (ii) a vegetable oil.

118. The pharmaceutical composition of claim 117, wherein the esters with propylene glycol comprise propylene glycol monolaurate, propylene glycol ricinoleate, propylene glycol monooleate, propylene glycol monocaprylate, propylene glycol dicaprylate/dicaprate, or propylene glycol dioctanoate.

119. The pharmaceutical composition of claim 28, wherein the at least one solubilizer further comprises a fatty acid having at least nine carbons.

120. The pharmaceutical composition of claim 119, wherein the fatty acid having at least nine carbons comprises lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linolelaidic acid, γ-linolenic acid, α-linolenic acid, stearidonic acid, paullinic acid, gondoic acid, dihomo-γ-linolenic acid, mead acid, arachidonic acid, eicosapentaenoic acid, erucic acid, docosatetraenoic acid, docosahexaenoic acid, nervonic acid, or any combinations thereof.

121. The pharmaceutical composition of claim 119, wherein the fatty acid having at least nine carbons is present in an amount of from about 1% to about 50? of the total weight of the pharmaceutical composition.

122. The pharmaceutical composition of claim 1, consisting of:
(a) clofazimine in an amount of about 0.01% to about 1% of the total weight of the pharmaceutical composition;
(b) petroleum jelly in an amount of about 70% to about 80% of the total weight of the pharmaceutical composition; and
(c) liquid paraffin in an amount of from about 0.5% to about 30% of the total weight of the pharmaceutical composition.
